SEQUENCE LISTING (9 sequences)

SEQ ID NO: 1 - DNA, 18 bp, Artificial
  primer sequence
ccagctgccctacttgga

SEQ ID NO: 2 - DNA, 20 bp, Artificial
  primer sequence
gtgtcccttgttgtgctgtg

SEQ ID NO: 3 - DNA, 8 bp, Artificial
  primer sequence
cctggaga

SEQ ID NO: 4 - DNA, 18 bp, Artificial
  primer sequence
acttcgggatgggaaagc

SEQ ID NO: 5 - DNA, 18 bp, Artificial
  primer sequence
gaggaaggtggggtccat

SEQ ID NO: 6 - DNA, 8 bp, Artificial
  primer sequence
tggaggag

SEQ ID NO: 7 - DNA, 21 bp, Artificial
  primer sequence
gatggctctcatcccagactt

SEQ ID NO: 8 - DNA, 20 bp, Artificial
  primer sequence
agtccatgtgaatgggttcc

SEQ ID NO: 9 - DNA, 8 bp, Artificial
  primer sequence
ttctcctg

(19) United States
(12) Patent Application Publication
(10) Pub. No.: US 2023/0277630 A1
(43) Pub. Date: Sep. 7, 2023

(54) METHODS AND COMPOSITIONS FOR TREATING AMYOTROPHIC LATERAL SCLEROSIS

(71) Applicant: Amylyx Pharmaceuticals, Inc., Cambridge, MA (US)

(72) Inventors: Joshua Cohen, Canton, MA (US); Justin Klee, Cambridge, MA (US)

(21) Appl. No.: 17/684,845

(22) Filed: Mar. 2, 2022

(51) Int. Cl. A61K 38/41; A61K 31/575; A61K 31/192; A61K 45/06

(52) U.S. Cl. CPC A61K 38/415; A61K 31/575; A61K 31/192; A61K 45/06

(57) ABSTRACT

Provided herein are methods for treating at least one symptom of ALS in a subject who has received a first dosage of a substrate of a CYP or a transporter, the method including administering to the subject a composition comprising a bile acid or a pharmaceutically acceptable salt thereof and a phenylbutyrate compound, monitoring the subject for response to the substrate, and administering a second dosage of the substrate, wherein the second dosage is less than the first dosage.

### SEQUENCE LISTING

This application contains a Sequence Listing that has been submitted electronically as an ASCII text file named 38709-0034001_ST25.txt. The ASCII text file, created on Jun. 21, 2022, is 1.83 kilobytes in size. The material in the ASCII text file is hereby incorporated by reference in its entirety.

### TECHNICAL FIELD

The present disclosure generally relates to compositions and methods for treating Amyotrophic lateral sclerosis.

### BACKGROUND

Amyotrophic lateral sclerosis (ALS) is the most prevalent progressive motor neuron disease. ALS causes the progressive degeneration of motor neurons, resulting in rapidly progressing muscle weakness and atrophy that eventually leads to partial or total paralysis. Median survival from symptom onset is 2 to 3 years, with respiratory failure being the predominant cause of death. ALS treatment currently centers on symptom management. Only two FDA-approved medications for ALS, riluzole and edaravone, are presently available. Accordingly, there is a need for improved therapies for treating ALS.

Concomitant administration of different drugs may lead to adverse effects since the metabolism and/or excretion of each drug may reduce or interfere with the metabolism and/or excretion of the other drug(s), thus increasing the effective concentrations of those drugs as compared to the effective concentrations of those drugs when administered alone. However, patients with neurodegenerative disease, such as ALS patients, often require treatment with multiple drugs, so that the potential toxicity of drug-drug interactions present disadvantages that can have deleterious consequences for these patients. Accordingly, improved methods of treatment allowing the administration of multiple drugs are desired.

### SUMMARY

Provided herein are methods of treating at least one symptom of Amyotrophic Lateral Sclerosis (ALS) in a subject, the method comprising: (a) administering to a subject who has received a first dosage of a substrate of a cytochrome P450 (CYP) an effective amount of a composition comprising about 1 gram of Taurursodiol (TURSO) and about 3 grams of sodium phenylbutyrate, (b) determining or having determined a first level of the substrate or a metabolite thereof in a first biological sample from the subject, and (c) administering to the subject a second dosage of the substrate, wherein the second dosage is lower than the first dosage.

In some embodiments, the CYP is CYP1A2, CYP2B6, CYP2C8, CYP2C9, CYP2C19, CYP2D6, or CYP3A.

In some embodiments, the CYP is CYP1A2. In some embodiments, the substrate is alosetron, caffeine, duloxetine, melatonin, ramelteon, tasimelteon, tizanidine, clozapine, pirfenidone, ramosetron, or theophylline.

In some embodiments, the CYP is CYP2B6. In some embodiments, the substrate is bupropion or efavirenz.

In some embodiments, the CYP is CYP2C8. In some embodiments, the substrate is repaglinide, montelukast, pioglitazone, or rosiglitazone.

In some embodiments, the CYP is CYP2C9. In some embodiments, the substrate is celecoxib, glimepiride, phenytoin, tolbutamide, or warfarin.

In some embodiments, the CYP is CYP2C19. In some embodiments, the substrate is S-mephenytoin, omeprazole, diazepam, lansoprazole, rabeprazole, or voriconazole.

In some embodiments, the CYP is 2D6. In some embodiments, the substrate is atomoxetine, desipramine, dextromethorphan, eliglustat(e), nebivolol, nortriptyline, perphenazine, tolterodine, R-venlafaxine, encainide, imipramine, metoprolol, propafenone, propranolol, tramadol, trimipramine, or S-venlafaxine.

In some embodiments, the CYP is CYP3A. In some embodiments, the substrate is alfentanil, avanafil, buspirone, conivaptan, cyclosporine, quinidine, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, tolvaptan, alprazolam, aprepitant, atorvastatin, colchicine, eliglustat, pimozide, rilpivirine, rivaroxaban, or tadalafil.

In some embodiments, the CYP is CYP3A4. In some embodiments, the substrate is alfentanil.

In some embodiments, the substrate is cyclosporine. In some embodiments, the first dosage of cyclosporine is about 0.5 to about 15 mg/kg/day. In some embodiments, the substrate is quinidine. In some embodiments, the substrate of the CYP is mexiletine, alfentanil, quinidine, cyclosporine, or warfarin. In some embodiments, the substrate of the CYP has a narrow therapeutic index.

In some embodiments, the method further comprises step (d), determining or having determined a second level of the substrate or the metabolite thereof in a second biological sample from the subject. In some embodiments, the second level of the substrate or the metabolite thereof is lower than the first level. In some embodiments, the biological sample is a plasma or serum sample.

Also provided herein are methods of treating at least one symptom of ALS in a subject, the method comprising: (a) administering to a subject who has received a first dosage of warfarin an effective amount of a composition comprising about 1 gram of TURSO and about 3 grams of sodium phenylbutyrate; (b) determining or having determined a first blood International Normalized Ratio (INR) level of the subject; and (c) administering to the subject a second dosage of warfarin, wherein the second dosage is lower than the first dosage.

In some embodiments, the first dosage of warfarin is about 0.5 to 12 mg/day.

In some embodiments, step (b) comprises determining or having determined the first blood INR level of the subject once daily. In some embodiments, step (b) comprises determining or having determined the first blood INR level of the subject once every one to four weeks. In some embodiments, the first blood INR level is about 3.0 or higher. In some embodiments, the first blood INR level is about 4.0 or higher.

In some embodiments, the method further comprises step (d), determining or having determined a second blood INR level of the subject. In some embodiments, the second blood INR level is lower than the first blood INR level.

Also provided herein are methods of treating at least one symptom of ALS in a subject, the method comprising: (a) administering to a subject who has received a first dosage of a narrow therapeutic index (NTI) drug an effective amount of a composition comprising about 1 gram of TURSO and about 3 grams of sodium phenylbutyrate, (b) determining or having determined a first level of the NTI drug or a metabolite thereof in a first biological sample from the subject, and (c) administering to the subject a second dosage of the NTI drug, wherein the second dosage is lower than the first dosage.

In some embodiments, the NTI drug is mexiletine, alfentanil, quinidine, cyclosporine, warfarin or digoxin.

In some embodiments, the method further comprises step (d), determining or having determined a second level of the NTI drug or the metabolite thereof in a second biological sample from the subject. In some embodiments, the second level of the NTI drug or the metabolite thereof is lower than the first level.

Also provided herein are methods of treating at least one symptom of ALS in a subject, the method comprising: (a) administering to a subject who has received a first dosage of a substrate of Organic Anion Transporter 1 (OAT1) an effective amount of a composition comprising about 1 gram of TURSO and about 3 grams of sodium phenylbutyrate; (b) determining or having determined a first level of the substrate or a metabolite thereof in a first biological sample from the subject; and (c) administering to the subject a second dosage of the substrate, wherein the second dosage is lower than the first dosage.

In some embodiments, the substrate of OAT1 is a penicillin, non-steroidal anti-inflammatory drug (NSAID), HIV protease inhibitor, or an antiviral drug. In some embodiments, the NSAID is diclofenac, ketoprofen, or methotrexate. In some embodiments, the antiviral drug is Adefovir, Cidofovir, or Tenofovir.

In some embodiments, the method further comprises step (d), determining or having determined a second level of the substrate or a metabolite thereof in a second biological sample from the subject. In some embodiments, the second level of the substrate or the metabolite thereof is lower than the first level. In some embodiments, the biological sample is a blood sample.

In some embodiments, the TURSO is administered at an amount of about 1 to about 2 grams per day, inclusive. In some embodiments, the sodium phenylbutyrate is administered at an amount of about 3 to about 6 grams per day, inclusive. In some embodiments, the TURSO is administered at an amount of about 1 gram once a day. In some embodiments, the TURSO is administered at an amount of about 1 gram twice a day. In some embodiments, the sodium phenylbutyrate is administered at an amount of about 3 grams once a day. In some embodiments, the sodium phenylbutyrate is administered at an amount of about 3 grams twice a day.

In some embodiments, the composition is administered to the subject orally or through a feeding tube. In some embodiments, the subject is diagnosed with ALS. In some embodiments, the subject is suspected as having ALS. In some embodiments, the subject is human.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

All publications, patent applications, patents, and other references mentioned herein are incorporated by reference in their entirety. In case of conflict, the present specification, including definitions, will control. In addition, the materials, methods, and examples are illustrative only and not intended to be limiting. Other features and advantages of the invention will be apparent from the following detailed description, and from the claims.

### BRIEF DESCRIPTION OF THE DRAWINGS

FIG. 1 is a set of graphs showing the inhibition of CYP activities by positive time-dependent inhibitors.

FIG. 2 shows individual Tenofovir plasma concentration (ng/mL) versus time data following oral administration of TDF only (Day 1, left panel) or AMX0035 and TDF (Day 5, right panel) in male Sprague Dawley rats.

FIG. 3 is a graph showing individual PB plasma concentration (ng/mL) versus time data following oral administration of AMX0035 and TDF in male Sprague Dawley rats (Day 5).

FIG. 4 shows individual TUDCA plasma concentration (ng/mL) versus time data following oral administration of TDF only (Day 1, left panel) or AMX0035 and TDF (Day 5, right panel) in male Sprague Dawley rats.

FIG. 5 shows mean Tenofovir, PB, and TUDCA plasma concentration (ng/mL) versus time data following oral administration of TDF only (Day 1, left panel) or AMX0035 and TDF (Day 5, right panel) in male Sprague Dawley rats.

FIG. 6 is a bar graph showing mean Tenofovir urine recovery (ng) data following oral administration of TDF only (Day 1, left) or AMX0035 and TDF (Day 5, right) in male Sprague Dawley rats.

### DETAILED DESCRIPTION

Applicant has discovered that a combination of a bile acid (e.g. Taurursodiol (TURSO)) and a phenylbutyrate compound (e.g. sodium phenylbutyrate) can be used for treating one or more symptoms of ALS, and has surprisingly found that the combination inhibits one or more cytochrome (CYP) P450 enzymes (e.g. CYP1A2, CYP2B6, CYP2C8, CYP2C9, CYP2C19, CYP2D6, and CYP3A) and transporters (e.g. Organic Anion Transporter 1 (OAT1)). When substrates of CYP P450 enzymes or transporters are administered concomitantly with a composition comprising a bile acid and a phenylbutyrate compound, the levels and/or effective dose of the substrates may be increased, which may result in an increase in any toxic effects associated with the substrates. Accordingly, the present disclosure provides methods of treating at least one symptom of ALS in a subject who is also receiving a substrate of CYP P450 enzymes (e.g. those that have a narrow therapeutic index) or a substrate of transporters (e.g. a substrate of OAT1).

The present disclosure provides methods of treating at least one symptom of ALS in a subject, the methods including (a) administering to a subject who has received a first dosage of a substrate of a cytochrome P450 enzyme an effective amount of a composition comprising about 1 gram of TURSO and about 3 grams of sodium phenylbutyrate, (b) determining or having determined a first level of the substrate in a first biological sample from the subject, and (c) administering to the subject a second dosage of the substrate, wherein the second dosage is lower than the first dosage.

The present disclosure also provides methods of treating at least one symptom of ALS in a subject, the methods including (a) administering to a subject who has received a first dosage of warfarin an effective amount of a composition comprising about 1 gram of TURSO and about 3 grams of sodium phenylbutyrate; (b) determining or having determined a first blood INR level of the subject; and (c) administering to the subject a second dosage of warfarin, wherein the second dosage is lower than the first dosage.

Also provided are method of treating at least one symptom of ALS in a subject, the methods including (a) administering to a subject who has received a first dosage of a substrate of Organic Anion Transporter 1 (OAT1) an effective amount of a composition comprising about 1 gram of TURSO and about 3 grams of sodium phenylbutyrate; (b) determining or having determined a first level of the substrate in a first biological sample from the subject; and (c) administering to the subject a second dosage of the substrate, wherein the second dosage is lower than the first dosage.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art.

### I. Amyotrophic Lateral Sclerosis (ALS)

The terms “amyotrophic lateral sclerosis” and “ALS” are used interchangeably herein, and include all of the classifications of ALS known in the art, including, but not limited to classical ALS (e.g., ALS that affects both lower and upper motor neurons), Primary Lateral Sclerosis (PLS, e.g., those that affect only the upper motor neurons), Progressive Bulbar Palsy (PBP or Bulbar Onset, a version of ALS that typically begins with difficulties swallowing, chewing and speaking) and Progressive Muscular Atrophy (PMA, typically affecting only the lower motor neurons). The terms include sporadic and familial (hereditary) ALS, ALS at any rate of progression (e.g., rapid, non-slow or slow progression) and ALS at any stage (e.g., prior to onset, at onset and late stages of ALS).

The subjects in the methods described herein may exhibit one or more symptoms associated with ALS, or have been diagnosed with ALS. In some embodiments, the subjects may be suspected as having ALS, and/or at risk for developing ALS.

The subjects in the methods described herein may exhibit one or more symptoms associated with benign fasciculation syndrome (BFS) or cramp-fasciculation syndrome (CFS).

Some embodiments of any of the methods described herein can further include determining that a subject has or is at risk for developing ALS, diagnosing a subject as having or at risk for developing ALS, or selecting a subject having or at risk for developing ALS. Likewise, some embodiments of any of the methods described herein can further include determining that a subject has or is at risk for developing benign fasciculation syndrome or cramp fasciculation syndrome, diagnosing a subject as having or at risk for developing BFS or CFS, or selecting a subject having or at risk for developing BFS or CFS.

In some embodiments of any of the methods described herein, the subject has shown one or more symptoms of ALS for about 24 months or less (e.g., about 23, 22, 21, 20, 19, 18, 17, 16, 15, 14, 13, 12, 11, 10, 9, 8, 7, 6, 5, 4, 3, 2, 1 month, or 1 week or less). In some embodiments, the subject has shown one or more symptoms of ALS for about 36 months or less (e.g., about 35, 34, 33, 32, 31, 30, 29, 28, 27, 26, or 25 months or less).

The order and type of ALS symptoms displayed by a subject may depend on which motor neurons in the body are damaged first, and consequently which muscles in the body are damaged first. For example, bulbar onset, limb onset, or respiratory onset ALS may present with similar or different symptoms. In general, ALS symptoms may include muscle weakness or atrophy (e.g., affecting upper body, lower body, and/or speech), muscle fasciculation (twitching), cramping, or stiffness of affected muscles. Early symptoms of ALS may include those of the arms or legs, difficulty in speaking clearly or swallowing (e.g., in bulbar onset ALS). Other symptoms include loss of tongue mobility, respiratory difficulties, difficulty breathing or abnormal pulmonary function, difficulty chewing, and/or difficulty walking (e.g., resulting in stumbling). Subjects may have respiratory muscle weakness as the initial manifestation of ALS symptoms. Such subjects may have very poor prognosis and in some instances have a median survival time of about two months from diagnosis. In some subjects, the time of onset of respiratory muscle weakness can be used as a prognostic factor.

ALS symptoms can also be classified by the part of the neuronal system that is degenerated, namely, upper motor neurons or lower motor neurons. Lower motor neuron degeneration manifests, for instance, as weakness or wasting in one or more of the bulbar, cervical, thoracic, and/or lumbosacral regions. Upper motor neuron degeneration can include increased tendon reflexes, spasticity, pseudo bulbar features, Hoffmann reflex, extensor plantar response, and exaggerated reflexes (hyperreflexia) including an overactive gag reflex. Progression of neuronal degeneration or muscle weakness is a hallmark of the disease. Accordingly, some embodiments of the present disclosure provide a method of ameliorating at least one symptom of lower motor neuron degeneration, at least one symptom of upper motor neuron degeneration, or at least one symptom from each of lower motor neuron degeneration and upper motor neuron degeneration. In some embodiments of any of the methods described herein, symptom onset can be determined based on information from subject and/or subject's family members. In some embodiments, the median time from symptom onset to diagnosis is about 12 months.

In some instances, the subject has been diagnosed with ALS. For example, the subject may have been diagnosed with ALS for about 24 months or less (e.g., about 23, 22, 21, 20, 19, 18, 17, 16, 15, 14, 13, 12, 11, 10, 9, 8, 7, 6, 5, 4, 3, 2, or 1 month or less). For example, the subject may have been diagnosed with ALS for 1 week or less, or on the same day that the presently disclosed treatments are administered. The subject may have been diagnosed with ALS for more than about 24 months (e.g., more than about 28, 32, 36, 40, 44, 48, 52, 56, 60, 64, 68, 72, 76, or 80 months). Methods of diagnosing ALS are known in the art. For example, the subject can be diagnosed based on clinical history, family history, physical or neurological examinations (e.g., signs of lower motor neuron or upper motor neuron degeneration). The subject can be confirmed or identified, e.g. by a healthcare professional, as having ALS. Multiple parties may be included in the process of diagnosis. For example, where samples are obtained from a subject as part of a diagnosis, a first party can obtain a sample from a subject and a second party can test the sample. In some embodiments of any of the human subjects described herein, the subject is diagnosed, selected, or referred by a medical practitioner (e.g., a general practitioner).

In some embodiments, the subject fulfills the El Escorial criteria for probable or definite ALS, i.e. the subject presents:
1. Signs of lower motor neuron (LMN) degeneration by clinical, electrophysiological or neuropathologic examination;
2. Signs of upper motor neuron (UMN) degeneration by clinical examination; and
3. Progressive spread of signs within a region or to other regions, together with the absence of:
Electrophysiological evidence of other disease processes that might explain the signs of LMN and/or UMN degenerations; and
Neuroimaging evidence of other disease processes that might explain the observed clinical and electrophysiological signs.

Under the El Escorial criteria, signs of LMN and UMN degeneration in four regions are evaluated, including brainstem, cervical, thoracic, and lumbrasacral spinal cord of the central nervous system. The subject may be determined to be one of the following categories:
A. Clinically Definite ALS, defined on clinical evidence alone by the presence of UMN, as well as LMN signs, in three regions.
B. Clinically Probable ALS, defined on clinical evidence alone by UMN and LMN signs in at least two regions with some UMN signs necessarily rostral to (above) the LMN signs.
C. Clinically Probable ALS—Laboratory-supported, defined when clinical signs of UMN and LMN dysfunction are in only one region, or when UMN signs alone are present in one region, and LMN signs defined by EMG criteria are present in at least two limbs, with proper application of neuroimaging and clinical laboratory protocols to exclude other causes.
D. Clinically Possible ALS, defined when clinical signs of UMN and LMN dysfunction are found together in only one region or UMN signs are found alone in two or more regions; or LMN signs are found rostral to UMN signs and the diagnosis of Clinically Probable—Laboratory-supported.

In some embodiments, the subject has clinically definite ALS (e.g., based on the El Escorial criteria).

The subject can be evaluated and/or diagnosed using the Revised Amyotrophic Lateral Sclerosis Functional Rating Scale (ALSFRS-R). The ALSFRS-R is an ordinal rating scale (ratings 0-4) used to determine subjects' assessment of their capability and independence in 12 functional activities relevant in ALS. ALSFRS-R scores calculated at diagnosis can be compared to scores throughout time to determine the speed of progression. Change in ALSFRS-R scores can be correlated with change in strength over time, and can be associated with quality of life measures and predicted survival. ALSFRS-R demonstrates a linear mean slope and can be used as a prognostic indicator (See e.g., Berry et al. Amyotroph Lateral Scler Frontotemporal Degener 15:1-8, 2014; Traynor et al., Neurology 63:1933-1935, 2004; Simon et al., Ann Neurol 76:643-657, 2014; and Moore et al. Amyotroph Lateral Scler Other Motor Neuron Disord 4:42, 2003).

In the ALSFRS-R, functions mediated by cervical, trunk, lumbosacral, and respiratory muscles are each assessed by 3 items. Each item is scored from 0-4, with 4 reflecting no involvement by the disease and 0 reflecting maximal involvement. The item scores are added to give a total. Total scores reflect the impact of ALS, with the following exemplary categorization: >40 (minimal to mild); 39-30 (mild to moderate); <30 (moderate to severe); <20 (advanced disease).

For example, a subject can have an ALSFRS-R score (e.g., a baseline ALSFRS-R score) of 40 or more (e.g., at least 41, 42, 43, 44, 45, 46, 47, or 48), between 30 and 39, inclusive (e.g., 31, 32, 33, 34, 35, 36, 37, or 38), or 30 or less (e.g., 21, 22, 23, 24, 25, 26, 27, 28, or 29). In some embodiments of any of the methods described herein, the subject has an ALSFRS-R score (e.g., a baseline ALSFRS-R score) of 40 or less (e.g., 39, 38, 37, 36, 35, 34, 33, 32, 31, 30, 29, 28, 27, 26, 25, 24, 23, 22, 21, 20, 19, 18, 17, 16, 15, 14, 13, 12, 11, 10 or less). In some embodiments, the subject has an ALSFRS-R score (e.g., a baseline ALSFRS-R score) of 20 or less (e.g., 19, 18, 17, 16, 15, 14, 13, 12, 11, 10, 9, 8, 7, 6, 5 or less).

As ALS is a progressive disease, all patients generally will progress over time. However, a large degree of inter-subject variability exists in the rate of progression, as some subjects die or require respiratory support within months while others have relatively prolonged survival. The subjects described herein may have rapid progression ALS or slow progression ALS. The rate of functional decline in a subject with ALS can be measured by the change in ALSFRS-R score per month. For example, the score can decrease by about 1.02 (±2.3) points per month.

One predictor of patient progression is the patient's previous rate of disease progression (ΔFS), which can be calculated as: ΔFS=(48−ALSFRS-R score at the time of evaluation)/duration from onset to time of evaluation (month). The ΔFS score represents the number of ALSFRS-R points lost per month since symptom onset, and can be a significant predictor of progression and/or survival in subjects with ALS (See e.g., Labra et al. J Neurol Neurosurg Psychiatry 87:628-632, 2016 and Kimura et al. Neurology 66:265-267, 2006). The subject may have a disease progression rate (ΔFS) of about 0.50 or less (e.g., about 0.45, 0.40, 0.35, 0.30, 0.25, 0.20, 0.15, or 0.10 or less); between about 0.50 and about 1.20 inclusive (e.g., about 0.55, 0.60, 0.65, 0.70, 0.75, 0.80, 0.85, 0.90, 0.95, 1.00, 1.05, 1.10, or 1.15); or about 1.20 or greater (e.g., about 1.25, 1.30, 1.35, 1.40, 1.45, 1.50, 1.55, 1.60, 1.75, 1.80, 1.85, 1.90, 1.95, or 2.00 or greater). In some embodiments of any of the methods described herein, the subject can have an ALS disease progression rate (ΔFS) of about 0.50 or greater (e.g., about 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.75, 1.80, 1.85, 1.90, 1.95, or 2.00 or greater). However, it should be noted that the ΔFS score is a predictor of patient progression, and may under or overestimate a patient's progression once under evaluation.

In some embodiments, since initial evaluation, the subject has lost on average about 0.8 to about 2 (e.g., about 0.9, 1.0, 1.1, 1.2, 1.3, 1.4, 1.5, 1.6, 1.7, 1.8, or 1.9) ALSFRS-R points per month over 3-12 months. In some embodiments, the subject has lost on average more than about 1.2 ALSFRS-R points per month over 3-12 months since initial evaluation. The subject may have had a decline of at least 3 points (e.g., at least 4, 6, 8, 10, 12, 14, 16, 20, 24, 28, or 32 points) in ALSFRS-R score over 3-12 months since initial evaluation. In some embodiments, the subject has lost on average about 0.8 to about 2 (e.g., about 0.9, 1.0, 1.1, 1.2, 1.3, 1.4, 1.5, 1.6, 1.7, 1.8, or 1.9) ALSFRS-R points per month over the previous 3-12 months. In some embodiments, the subject has lost on average more than about 1.2 (e.g., more than about 1.5, 1.8, 2.0, 2.5, or 3) ALSFRS-R points per month over the previous 3-12 months.

In some embodiments of any of the methods described herein, the presence or level of a marker in a sample obtained from the subject may be used for ALS diagnosis or prognosis, or to track disease activity and treatment responses. Suitable samples include, for example, cells, tissues, or body fluids (e.g. blood, urine, or cerebral spinal fluid (CSF) samples). For instance, levels of phosphorylated neurofilament heavy subunit (pNF-H) or neurofilament light chain (NfL) in the CSF and/or blood can be used as a biomarker for ALS diagnosis, prognosis, or to track disease activity or treatment outcomes. pNF-H is a main component of the neuronal cytoskeleton and is released into the CSF and the bloodstream with neuronal damage. Levels of pNF-H may correlate with the level of axonal loss and/or burden of motor neuron dysfunction (See, e.g., De Schaepdryver et al. Journal of Neurology, Neurosurgery & Psychiatry 89:367-373, 2018).

The concentration of pNF-H in the CSF and/or blood of a subject with ALS may significantly increase in the early disease stage. Higher levels of pNF-H in the plasma, serum and/or CSF may be associated with faster ALS progression (e.g., faster decline in ALSFRS-R), and/or shorter survival. pNF-H concentration in plasma may be higher in ALS subjects with bulbar onset than those with spinal onset. In some cases, an imbalance between the relative expression levels of the neurofilament heavy and light chain subunits can be used for ALS diagnosis, prognosis, or tracking disease progression.

Methods of detecting pNF-H and NfL (for example, in the cerebrospinal fluid, plasma, or serum) are known in the art and include but are not limited to, ELISA and Simoa assays (See e.g., Shaw et al. Biochemical and Biophysical Research Communications 336:1268-1277, 2005; Ganesalingam et al. Amyotroph Lateral Scler Frontotemporal Degener 14(2):146-9, 2013; De Schaepdryver et al. Annals of Clinical and Translational Neurology 6(10): 1971-1979, 2019; Wilke et al. Clin Chem Lab Med 57(10):1556-1564, 2019; Poesen et al. Front Neurol 9:1167, 2018; Pawlitzki et al. Front. Neurol. 9:1037, 2018; Gille et al. Neuropathol Appl Neurobiol 45(3):291-304, 2019). Commercialized pNF-H detection assays can also be used, such as those developed by EnCor Biotechnology, BioVendor, and Millipore-EMD. Commercial NfL assay kits based on the Simoa technology, such as those produced by Quanterix can also be used (See, e.g., Thouvenot et al. European Journal of Neurology 27:251-257, 2020). Factors affecting pNF-H and NfL levels or their detection in serum or plasma in relation to disease course may differ from those in CSF. The levels of neurofilament (e.g. pNF-H and/or NfL) in the CSF and serum may be correlated (See, e.g., Wilke et al. Clin Chem Lab Med 57(10):1556-1564, 2019).

Subjects described herein may have a CSF or blood pNF-H level of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher). In some embodiments, the serum pNF-H level can be about 70 to about 1200 pg/mL (e.g., about 70 to about 1000, about 70 to about 800, about 80 to about 600, or about 90 to about 400 pg/mL). In some embodiments, the CSF pNF-H level can be about 1000 to about 5000 pg/mL (e.g., about 1500 to about 4000, or about 2000 to about 3000 pg/mL).

The subjects may have a CSF or blood level of NfL of about 50 pg/mL or higher (e.g., about 60, 70, 80, 90, 100, 110, 120, 130, 140, 150, 160, 170, 180, 190, 200, 210, 220, 230, 240, or 250 pg/mL or higher). In some embodiments, the serum NfL level can be about 50 to about 300 pg/mL (e.g., about 50 to about 280, about 50 to about 250, about 50 to about 200, about 50 to about 150, about 50 to about 100, about 100 to about 300, about 100 to about 250, about 100 to about 200, about 100 to about 150, about 150 to about 300, about 150 to about 250, about 150 to about 200, about 200 to about 300, about 200 to about 250, or about 250 to about 300 pg/mL). In some embodiments, the CSF NfL level can be about 2000 to about 40,000 pg/mL (e.g., about 2000 to about 35,000, about 2000 to about 30,000, about 2000 to about 25,000, about 2000 to about 20,000, about 2000 to about 15,000, about 2000 to about 10,000, about 2000 to about 8000, about 2000 to about 6000, about 2000 to about 4000, about 4000 to about 40,000, about 4000 to about 35,000, about 4000 to about 30,000, about 4000 to about 25,000, about 4000 to about 20,000, about 4000 to about 15,000, about 4000 to about 10,000, about 4000 to about 8000, about 4000 to about 6000, about 6000 to about 40,000, about 6000 to about 35,000, about 6000 to about 30,000, about 6000 to about 25,000, about 6000 to about 20,000, about 6000 to about 15,000, about 6000 to about 10,000, about 6000 to about 8000, about 8000 to about 40,000, about 8000 to about 35,000, about 8000 to about 30,000, about 8000 to about 25,000, about 8000 to about 20,000, about 8000 to about 15,000, about 8000 to about 10,000, about 10,000 to about 40,000, about 10,000 to about 35,000, about 10,000 to about 30,000, about 10,000 to about 25,000, about 10,000 to about 20,000, about 10,000 to about 15,000, about 15,000 to about 40,000, about 15,000 to about 35,000, about 15,000 to about 30,000, about 15,000 to about 25,000, about 15,000 to about 20,000, about 20,000 to about 40,000, about 20,000 to about 35,000, about 20,000 to about 30,000, about 20,000 to about 25,000, about 25,000 to about 40,000, about 25,000 to about 35,000, about 25,000 to about 30,000, about 30,000 to about 40,000, about 30,000 to about 35,000, or about 35,000 to about 40,000 pg/mL).

Additional biomarkers useful for ALS diagnosis, prognosis, and disease progression monitoring are contemplated herein, including but are not limited to, CSF levels of S100-β, cystatin C, and chitotriosidase (CHIT) (See e.g., Chen et al. BMC Neurol 16:173, 2016). Serum levels of uric acid can be used as a biomarker for prognosing ALS (See e.g., Atassi et al. Neurology 83(19):1719-1725, 2014). Akt phosphorylation can also be used as a biomarker for prognosing ALS (See e.g., WO2012/160563). Urine levels of p75ECD and ketones can be used as a biomarker for ALS diagnosis (See e.g., Shepheard et al. Neurology 88:1137-1143, 2017). Serum and urine levels of creatinine can also be used as a biomarker. Other useful blood, CSF, neurophysiological, and neuroradiological biomarkers for ALS are described in e.g., Turner et al. Lancet Neurol 8:94-109, 2009. Any of the markers described herein can be used for diagnosing a subject as having ALS, or determining that a subject is at risk for developing ALS.

A subject may also be identified as having ALS, or at risk for developing ALS, based on genetic analysis. Genetic variants associated with ALS are known in the art (See, e.g., Taylor et al. Nature 539:197-206, 2016; Brown and Al-Chalabi N Engl J Med 377:162-72, 2017; and http://alsod.iop.kcl.ac.uk). Subjects described herein can carry mutations in one or more genes associated with familial and/or sporadic ALS. Exemplary genes associated with ALS include but are not limited to: ANG, TARDBP, VCP, VAPB, SQSTM1, DCTN1, FUS, UNC13A, ATXN2, HNRNPA1, CHCHD10, MOBP, C21ORF2, NEK1, TUBA4A, TBK1, MATR3, PFN1, UBQLN2, TAF15, OPTN, TDP-43, and DAO. Additional description of genes associated with ALS can be found at Therrien et al. Curr Neurol Neurosci Rep 16:59-71, 2016; Peters et al. J Clin Invest 125:2548, 2015, and Pottier et al. J Neurochem, 138: Suppl 1:32-53, 2016. Genetic variants associated with ALS can affect the ALS progression rate in a subject, the pharmacokinetics of the administered compounds in a subject, and/or the efficacy of the administered compounds for a subject.

The subjects may have a mutation in the gene encoding CuZn-Superoxide Dismutase (SOD1). Mutation causes the SOD1 protein to be more prone to aggregation, resulting in the deposition of cellular inclusions that contain misfolded SOD1 aggregates (See e.g., Andersen et al., Nature Reviews Neurology 7:603-615, 2011). Over 100 different mutations in SOD1 have been linked to inherited ALS, many of which result in a single amino acid substitution in the protein. In some embodiments, the SOD1 mutation is A4V (i.e., a substitution of valine for alanine at position 4). SOD1 mutations are further described in, e.g., Rosen et al. Hum. Mol. Genet. 3, 981-987, 1994 and Rosen et al. Nature 362:59-62, 1993. In some embodiments, the subject has a mutation in the C9ORF72 gene. Repeat expansions in the C9ORF72 gene are a frequent cause of ALS, with both loss of function of C9ORF72 and gain of toxic function of the repeats being implicated in ALS (See e.g., Balendra and Isaacs, Nature Reviews Neurology 14:544-558, 2018).

The methods described herein can include, prior to administration of a bile acid and a phenylbutyrate compound, detecting a SOD1 mutations and/or a C9ORF72 mutation in the subject. Methods for screening for mutations are well known in the art. Suitable methods include, but are not limited to, genetic sequencing. See, e.g., Hou et al. Scientific Reports 6: 32478, 2016; and Vajda et al. Neurology 88:1-9, 2017.

Skilled practitioners will appreciate that certain factors can affect the bioavailability and metabolism of the administered compounds for a subject, and can make adjustments accordingly. These include but are not limited to liver function (e.g. levels of liver enzymes), renal function, and gallbladder function (e.g., ion absorption and secretion, levels of cholesterol transport proteins). There can be variability in the levels of exposure each subject has for the administered compounds (e.g., bile acid and a phenylbutyrate compound), differences in the levels of excretion, and in the pharmacokinetics of the compounds in the subjects being treated. Any of the factors described herein may affect drug exposure by the subject. For instance, decreased clearance of the compounds can result in increased drug exposure, while improved renal function can reduce the actual drug exposure. The extent of drug exposure may be correlated with the subject's response to the administered compounds and the outcome of the treatment.

The subject can be e.g., older than about 18 years of age (e.g., between 18-100, 18-90, 18-80, 18-70, 18-60, 18-50, 18-40, 18-30, 18-25, 25-100, 25-90, 25-80, 25-70, 25-60, 25-50, 25-40, 25-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 years of age). The subject can have a BMI of between about 18.5-30 kg/m² (e.g., between 18.5-28, 18.5-26, 18.5-24, 18.5-22, 18.5-20, 20-30, 20-28, 20-26, 20-24, 20-22, 22-30, 22-28, 22-26, 22-24, 24-30, 24-28, 24-26, 26-30, 26-28, or 28-30 kg/m²). Having a mutation in any of the ALS-associated genes described herein or presenting with any of the biomarkers described herein may suggest that a subject is at risk for developing ALS. Such subjects can be treated with the methods provided herein for preventative and prophylaxis purposes.

In some embodiments, the subjects have one or more symptoms of benign fasciculation syndrome (BFS) or cramp-fasciculation syndrome (CFS). BFS and CFS are peripheral nerve hyperexcitability disorders, and can cause fasciculation, cramps, pain, fatigue, muscle stiffness, and paresthesia. Methods of identifying subjects with these disorders are known in the art, such as by clinical examination and electromyography.

### II. Composition

The present disclosure provides methods of treating at least one symptom of ALS in a subject, the methods including administering to the subject a bile acid or a pharmaceutically acceptable salt thereof and a phenylbutyrate compound. In some embodiments, the methods include administering a composition comprising a TURSO and a sodium phenylbutyrate to a subject.

### Bile Acid

As used herein, “bile acid” refers to naturally occurring surfactants having a nucleus derived from cholanic acid substituted with a 3α-hydroxyl group and optionally with other hydroxyl groups as well, typically at the C6, C7 or C12 position of the sterol nucleus. Bile acid derivatives (e.g., aqueous soluble bile acid derivatives) and bile acids conjugated with an amine are also encompassed by the term “bile acid”. Bile acid derivatives include, but are not limited to, derivatives formed at the carbon atoms to which hydroxyl and carboxylic acid groups of the bile acid are attached with other functional groups, including but not limited to halogens and amino groups. Soluble bile acids may include an aqueous preparation of a free acid form of bile acids combined with one of HCl, phosphoric acid, citric acid, acetic acid, ammonia, or arginine. Suitable bile acids include but are not limited to, taurursodiol (TURSO), ursodeoxycholic acid (UDCA), chenodeoxycholic acid (also referred to as “chenodiol” or “chenic acid”), cholic acid, hyodeoxycholic acid, deoxycholic acid, 7-oxolithocholic acid, lithocholic acid, iododeoxycholic acid, iocholic acid, taurochenodeoxycholic acid, taurodeoxycholic acid, glycoursodeoxycholic acid, taurocholic acid, glycocholic acid, or an analog, derivative, or prodrug thereof.

In some embodiments, the bile acids of the present disclosure are hydrophilic bile acids. Hydrophilic bile acids include but are not limited to, TURSO, UDCA, chenodeoxycholic acid, cholic acid, hyodeoxycholic acid, lithocholic acid, and glycoursodeoxycholic acid.

Pharmaceutically acceptable salts or solvates of any of the bile acids disclosed herein are also contemplated. In some embodiments, bases commonly employed to form pharmaceutically acceptable salts of the bile acids of the present disclosure include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The terms “tauroursodeoxycholic acid” (TUDCA) and “taurursodiol” (TURSO) are used interchangeably herein.

The bile acid described herein can be TURSO, as shown in formula I (with labeled carbons to assist in understanding where substitutions may be made).

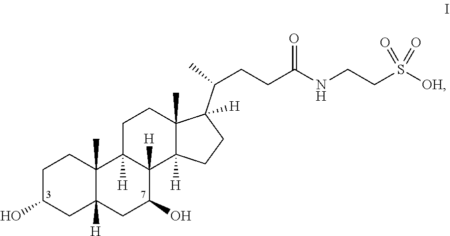

or a pharmaceutically acceptable salt thereof.

The bile acid described herein can be UDCA as shown in formula II (with labeled carbons to assist in understanding where substitutions may be made).

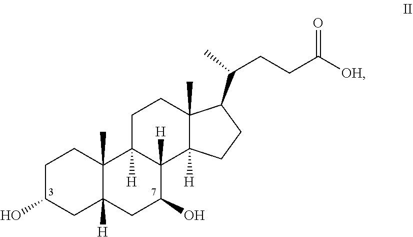

or a pharmaceutically acceptable salt thereof.

Derivatives of bile acids of the present disclosure can be physiologically related bile acid derivatives. For example, any combination of substitutions of hydrogen at position 3 or 7, a shift in the stereochemistry of the hydroxyl group at positions 3 or 7, in the formula of TURSO or UDCA are suitable for use in the present composition.

The “bile acid” can also be a bile acid conjugated with an amino acid. The amino acid in the conjugate can be, but are not limited to, taurine, glycine, glutamine, asparagine, methionine, or carbocysteine. Other amino acids that can be conjugated with a bile acid of the present disclosure include arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid. One example of such a bile acid is a compound of formula III:

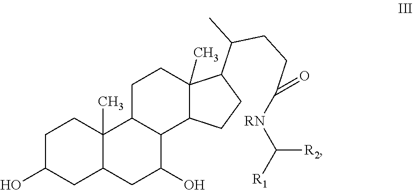

wherein
R is —H or C₁-C₄ alkyl;
R₁ is —CH₂—SO₃R₃, CH₂COOH, or CH₂CH₂COOH, and R₂ is —H;
or R₁ is —COOH and R₂ is —CH₂—CH₂—CONH₂, —CH₂—CONH₂, —CH₂—CH₂—SCH₃, CH₂CH₂CH₂NH(C═NH)NH₂, CH₂(imidazolyl), CH₂CH₂CH₂CH₂NH₂, CH₂COOH, CH₂CH₂COOH, CH₂OH, CH(OH)CH₃, CH₂SH, pyrrolidin-2-yl, CH₃, 2-propyl, 2-butyl, 2-methylbutyl, CH₂(phenyl), CH₂(4-OH-phenyl), or —CH₂—S—CH₂—COOH; and
R₃ is —H or the residue of an amino acid, or a pharmaceutically acceptable analog, derivative, prodrug thereof, or a mixture thereof. One example of the amino acid is a basic amino acid. Other examples of the amino acid include glycine, glutamine, asparagine, methionine, carbocysteine, arginine, histidine, lysine, aspartic acid, glutamic acid, serine, threonine, cysteine, proline, alanine, valine, isoleucine, leucine, phenylalanine, tyrosine, and tryptophan, as well as β-alanine, and γ-aminobutyric acid.

Another example of a bile acid of the present disclosure is a compound of formula IV:

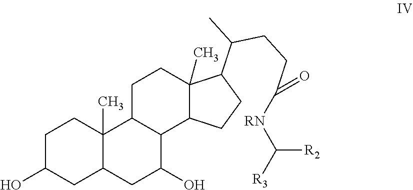

wherein
R is —H or C₁-C₄ alkyl;
R₁ is —CH₂—SO₃R₃, and R₂ is —H;
or R₁ is —COOH and R₂ is —CH₂—CH₂—CONH₂, —CH₂—CONH₂, —CH₂—CH₂—SCH₃, or —CH₂—S—CH₂—COOH; and
R₃ is —H or the residue of a basic amino acid, or a pharmaceutically acceptable analog, derivative, prodrug thereof, or a mixture thereof. Examples of basic amino acids include lysine, histidine, and arginine.

In some embodiments, the bile acid is TURSO. TURSO is an ambiphilic bile acid and is the taurine conjugate form of UDCA. TURSO recovers mitochondrial bioenergetic deficits through incorporating into the mitochondrial membrane, reducing Bax translocation to the mitochondrial membrane, reducing mitochondrial permeability, and increasing the apoptotic threshold of the cell (Rodrigues et al. Biochemistry 42, 10: 3070-3080, 2003). It is used for the treatment of cholesterol gallstones, where long periods of treatment is generally required (e.g., 1 to 2 years) to obtain complete dissolution. It has been used for the treatment of cholestatic liver diseases including primary cirrhosis, pediatric familial intrahepatic cholestasis and primary sclerosing cholangitis and cholestasis due to cystic fibrosis. TURSO is contraindicated in subjects with biliary tract infections, frequent biliary colic, or in subjects who have trouble absorbing bile acids (e.g. ileal disease or resection). Drug interactions may include with substances that inhibit the absorption of bile acids, such as cholestyramine, and with drugs that increase the elimination of cholesterol in the bile (TURSO reduces biliary cholesterol content). Based on similar physicochemical characteristics, similarities in drug toxicity and interactions exist between TURSO and UDCA. The most common adverse reactions reported with the use of TURSO (≥1%) are: abdominal discomfort, abdominal pain, diarrhea, nausea, pruritus, and rash. There are some cases of pruritus and a limited number of cases of elevated liver enzymes.

In some embodiments, the bile acid is UDCA. UDCA, or ursodiol, has been used for treating gallstones, and is produced and secreted endogenously by the liver as a taurine (TURSO) or glycine (GUDCA) conjugate. Taurine conjugation increases the solubility of UDCA by making it more hydrophilic. TURSO is taken up in the distal ileum under active transport and therefore likely has a slightly a longer dwell time within the intestine than UDCA which is taken up more proximally in the ileum. Ursodiol therapy has not been associated with liver damage.

Abnormalities in liver enzymes have not been associated with Actigall® (Ursodiol USP capsules) therapy and, Actigall® has been shown to decrease liver enzyme levels in liver disease. However, subjects given Actigall® should have SGOT (AST) and SGPT (ALT) measured at the initiation of therapy and thereafter as indicated by the particular clinical circumstances. Previous studies have shown that bile acid sequestering agents such as cholestyramine and colestipol may interfere with the action of ursodiol by reducing its absorption. Aluminum-based antacids have been shown to adsorb bile acids in vitro and may be expected to interfere with ursodiol in the same manner as the bile acid sequestering agents. Estrogens, oral contraceptives, and clofibrate (and perhaps other lipid-lowering drugs) increase hepatic cholesterol secretion, and encourage cholesterol gallstone formation and hence may counteract the effectiveness of ursodiol.

### Phenylbutyrate Compounds

Phenylbutyrate compound is defined herein as encompassing phenylbutyrate (a low molecular weight aromatic carboxylic acid) as a free acid (4-phenylbutyrate (4-PBA), 4-phenylbutyric acid, or phenylbutyric acid), and pharmaceutically acceptable salts, co-crystals, polymorphs, hydrates, solvates, conjugates, derivatives or pro-drugs thereof. Phenylbutyrate compounds described herein also encompass analogs of 4-PBA, including but not limited to Glyceryl Tri-(4-phenylbutyrate), phenylacetic acid (which is the active metabolite of PBA), 2-(4-Methoxyphenoxy) acetic acid (2-POAA-OMe), 2-(4-Nitrophenoxy) acetic acid (2-POAA-NO2), and 2-(2-Naphthyloxy) acetic acid (2-NOAA), and their pharmaceutically acceptable salts. Phenylbutyrate compounds also encompass physiologically related 4-PBA species, such as but not limited to any substitutions for Hydrogens with Deuterium in the structure of 4-PBA. Other HDAC2 inhibitors are contemplated herein as substitutes for phenylbutyrate compounds.

Physiologically acceptable salts of phenylbutyrate, include, for example sodium, potassium, magnesium or calcium salts. Other example of salts include ammonium, zinc, or lithium salts, or salts of phenylbutyrate with an orgain amine, such as lysine or arginine.

In some embodiments of any of the methods described herein, the phenylbutyrate compound is sodium phenylbutyrate. Sodium phenylbutyrate has the following formula:

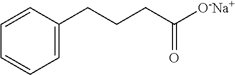

Phenylbutyrate is a pan-HDAC inhibitor and can ameliorate ER stress through upregulation of the master chaperone regulator DJ-1 and through recruitment of other chaperone proteins (See e.g., Zhou et al. J Biol Chem. 286: 14941-14951, 2011 and Suaud et al. JBC. 286:21239-21253, 2011). The large increase in chaperone production reduces activation of canonical ER stress pathways, folds misfolded proteins, and has been shown to increase survival in in vivo models including the G93A SOD1 mouse model of ALS (See e.g., Ryu, H et al. J Neurochem. 93:1087-1098, 2005).

In some embodiments, the combination of a bile acid (e.g., TURSO), or a pharmaceutically acceptable salt thereof, and a phenylbutyrate compound (e.g., sodium phenylbutyrate) has synergistic efficacy when dosed in particular ratios (e.g., any of the ratios described herein), in treating one or more symptoms associated with ALS. The combination can, for example, induce a mathematically synergistic increase in neuronal viability in a strong oxidative insult model (H₂O₂-mediated toxicity) by linear modeling, through the simultaneous inhibition of endoplasmic reticulum stress and mitochondrial stress (See, e.g. U.S. Pat. Nos. 9,872,865 and 10,251,896).

### Formulation

Bile acids and phenylbutyrate compounds described herein can be formulated for use as or in pharmaceutical compositions. For example, the methods described herein can include administering an effective amount of a composition comprising TURSO and sodium phenylbutyrate. The term “effective amount”, as used herein, refer to an amount or a concentration of one or more drugs for a period of time (including acute or chronic administration and periodic or continuous administration) that is effective within the context of its administration for causing an intended effect or physiological outcome. The composition can include about 5% to about 15% w/w (e.g., about 6% to about 14%, about 7% to about 13%, about 8% to about 12%, about 8% to about 11%, about 9% to about 10%, or about 9.7% w/w) of TURSO and about 15% to about 45% w/w (e.g., about 20% to about 40%, about 25% to about 35%, about 28% to about 32%, or about 29% to about 30%, e.g., about 29.2% w/w) of sodium phenylbutyrate. In some embodiments, the composition includes about 9.7% w/w of TURSO and 29.2% w/w of sodium phenylbutyrate.

The sodium phenylbutyrate and TURSO can be present in the composition at a ratio by weight of between about 1:1 to about 4:1 (e.g., about 2:1 or about 3:1). In some embodiments, the ratio between sodium phenylbutyrate and TURSO is about 3:1.

The compositions described herein can include any pharmaceutically acceptable carrier, adjuvant, and/or vehicle. The term “pharmaceutically acceptable carrier or adjuvant” refers to a carrier or adjuvant that may be administered to a patient, together with a compound disclosed herein, and which does not destroy the pharmacological activity thereof and is nontoxic when administered in doses sufficient to deliver a therapeutic amount of the compound. As used herein the language “pharmaceutically acceptable carrier” includes saline, solvents, dispersion media, coatings, antibacterial and antifungal agents, isotonic and absorption delaying agents, and the like, compatible with pharmaceutical administration. The pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

Compositions of the present disclosure can include about 8% to about 24% w/w of dextrates (e.g., about 9% to about 23%, about 10% to about 22%, about 10% to about 20%, about 11% to about 21%, about 12% to about 20%, about 13% to about 19%, about 14% to about 18%, about 14% to about 17%, about 15% to about 16%, or about 15.6% w/w of dextrates). Both anhydrous and hydrated dextrates are contemplated herein. The dextrates of the present disclosure can include a mixture of saccharides developed from controlled enzymatic hydrolysis of starch. Some embodiments of any of the compositions described herein include hydrated dextrates (e.g., NF grade, obtained from JRS Pharma, Colonial Scientific, or Quadra).

Compositions of the present disclosure can include about 1% to about 6% w/w of sugar alcohol (e.g., about 2% to about 5%, about 3% to about 4%, or about 3.9% w/w of sugar alcohol). Sugar alcohols can be derived from sugars and contain one hydroxyl group (—OH) attached to each carbon atom. Both disaccharides and monosaccharides can form sugar alcohols. Sugar alcohols can be natural or produced by hydrogenation of sugars. Exemplary sugar alcohols include but are not limited to, sorbitol, xylitol, and mannitol. In some embodiments, the composition comprises about 1% to about 6% w/w (e.g., about 2% to about 5%, about 3% to about 4%, or about 3.9% w/w) of sorbitol.

Compositions of the present disclosure can include about 22% to about 35% w/w of maltodextrin (e.g., about 22% to about 33%, about 24% to about 31%, about 25% to about 32%, about 26% to about 30%, or about 28% to about 29% w/w, e.g., about 28.3% w/w of maltodextrin). Maltodextrin can form a flexible helix enabling the entrapment of the active ingredients (e.g., any of the phenylbutyrate compounds and bile acids described herein) when solubilized into solution, thereby masking the taste of the active ingredients. Maltodextrin produced from any suitable sources are contemplated herein, including but not limited to, pea, rice, tapioca, corn, and potato. In some embodiments, the maltodextrin is pea maltodextrin. In some embodiments, the composition includes about 28.3% w/w of pea maltodextrin. For example, pea maltodextrin obtained from Roquette (KLEPTOSE® LINECAPS) can be used.

The compositions described herein can further include sugar substitutes (e.g. sucralose). For example, the compositions can include about 0.5% to about 5% w/w of sucralose (e.g., about 1% to about 4%, about 1% to about 3%, or about 1% to about 2%, e.g., about 1.9% w/w of sucralose). Other sugar substitutes contemplated herein include but are not limited to aspartame, neotame, acesulfame potassium, saccharin, and advantame.

In some embodiments, the compositions include one or more flavorants. The compositions can include about 2% to about 15% w/w of flavorants (e.g., about 3% to about 13%, about 3% to about 12%, about 4% to about 9%, about 5% to about 10%, or about 5% to about 8%, e.g., about 7.3% w/w). Flavorants can include substances that give another substance flavor, or alter the characteristics of a composition by affecting its taste. Flavorants can be used to mask unpleasant tastes without affecting physical and chemical stability, and can be selected based on the taste of the drug to be incorporated. Suitable flavorants include but are not limited to natural flavoring substances, artificial flavoring substances, and imitation flavors. Blends of flavorants can also be used. For example, the compositions described herein can include two or more (e.g., two, three, four, five or more) flavorants. Flavorants can be soluble and stable in water. Selection of suitable flavorants can be based on taste testing. For example, multiple different flavorants can be added to a composition separately, which are subjected to taste testing. Exemplary flavorants include any fruit flavor powder (e.g., peach, strawberry, mango, orange, apple, grape, raspberry, cherry or mixed berry flavor powder). The compositions described herein can include about 0.5% to about 1.5% w/w (e.g., about 1% w/w) of a mixed berry flavor powder and/or about 5% to about 7% w/w (e.g., about 6.3% w/w) of a masking flavor. Suitable masking flavors can be obtained from e.g., Firmenich.

The compositions described herein can further include silicon dioxide (or silica). Addition of silica to the composition can prevent or reduce agglomeration of the components of the composition. Silica can serve as an anti-caking agent, adsorbent, disintegrant, or glidant. In some embodiments, the compositions described herein include about 0.1% to about 2% w/w of porous silica (e.g., about 0.3% to about 1.5%, about 0.5% to about 1.2%, or about 0.8% to about 1%, e.g., 0.9% w/w). Porous silica may have a higher H₂O absorption capacity and/or a higher porosity as compared to fumed silica, at a relative humidity of about 20% or higher (e.g., about 25%, 30%, 35%, 40%, 45%, 50%, 55%, 60%, 65%, 70%, 75%, 80%, 85%, 90%, or 95% or higher). The porous silica can have an H₂O absorption capacity of about 5% to about 40% (e.g. about 20% to about 40%, or about 30% to about 40%) by weight at a relative humidity of about 50%. The porous silica can have a higher porosity at a relative humidity of about 20% or higher (e.g., about 30%, 40%, 50%, 60%, 70%, 80%, 90% or higher) as compared to that of fumed silica. In some embodiments, the porous silica have an average particle size of about 2 μm to about 10 μm (e.g. about 3 μm to about 9 μm, about 4 μm to about 8 μm, about 5 μm to about 8 μm, or about 7.5 μm). In some embodiments, the porous silica have an average pore volume of about 0.1 cc/gm to about 2.0 cc/gm (e.g., about 0.1 cc/gm to about 1.5 cc/gm, about 0.1 cc/gm to about 1 cc/gm, about 0.2 cc/gm to about 0.8 cc/gm, about 0.3 cc/gm to about 0.6 cc/gm, or about 0.4 cc/gm). In some embodiments, the porous silica have a bulk density of about 50 g/L to about 700 g/L (e.g. about 100 g/L to about 600 g/L, about 200 g/L to about 600 g/L, about 400 g/L to about 600 g/L, about 500 g/L to about 600 g/L, about 540 g/L to about 580 g/L, or about 560 g/L). In some embodiments, the compositions described herein include about 0.05% to about 2% w/w (e.g., any subranges of this range described herein) of Syloid® 63FP (WR Grace).

The compositions described herein can further include one or more buffering agents. For example, the compositions can include about 0.5% to about 5% w/w of buffering agents (e.g., about 1% to about 4% w/w, about 1.5% to about 3.5% w/w, or about 2% to about 3% w/w, e.g. about 2.7% w/w of buffering agents). Buffering agents can include weak acid or base that maintain the acidity or pH of a composition near a chosen value after addition of another acid or base. Suitable buffering agents are known in the art. In some embodiments, the buffering agent in the composition provided herein is a phosphate, such as a sodium phosphate (e.g., sodium phosphate dibasic anhydrous). For example, the composition can include about 2.7% w/w of sodium phosphate dibasic.

The compositions can also include one or more lubricants. For example, the compositions can include about 0.05% to about 1% w/w of lubricants (e.g., about 0.1% to about 0.9%, about 0.2% to about 0.8%, about 0.3% to about 0.7%, or about 0.4% to about 0.6%, e.g. about 0.5% w/w of lubricants). Exemplary lubricants include, but are not limited to sodium stearyl fumarate, magnesium stearate, stearic acid, metallic stearates, talc, waxes and glycerides with high melting temperatures, colloidal silica, polyethylene glycols, alkyl sulphates, glyceryl behenate, and hydrogenated oil. Additional lubricants are known in the art. In some embodiments, the composition includes about 0.05% to about 1% w/w (e.g., any of the subranges of this range described herein) of sodium stearyl fumarate. For example, the composition can include about 0.5% w/w of sodium stearyl fumarate.

In some embodiments, the composition include about 29.2% w/w of sodium phenylbutyrate, about 9.7% w/w of TURSO, about 15.6% w/w of dextrates, about 3.9% w/w of sorbitol, about 1.9% w/w of sucralose, about 28.3% w/w of maltodextrin, about 7.3% w/w of flavorants, about 0.9% w/w of silicon dioxide, about 2.7% w/w of sodium phosphate (e.g. sodium phosphate dibasic), and about 0.5% w/w of sodium stearyl fumerate.

The composition can include about 3000 mg of sodium phenylbutyrate, about 1000 mg of TURSO, about 1600 mg of dextrates, about 400 mg of sorbitol, about 200 mg of sucralose, about 97.2 mg of silicon dioxide, about 2916 mg of maltodextrin, about 746 mg of flavorants (e.g. about 102 mg of mixed berry flavor and about 644 mg of masking flavor), about 280 mg of sodium phosphate (e.g. sodium phosphate dibasic), and about 48.6 mg of sodium stearyl fumerate.

Additional suitable sweeteners or taste masking agents can also be included in the compositions, such as but not limited to, xylose, ribose, glucose, mannose, galactose, fructose, dextrose, sucrose, maltose, steviol glycosides, partially hydrolyzed starch, and corn syrup solid. Water soluble artificial sweeteners are contemplated herein, such as the soluble saccharin salts (e.g., sodium or calcium saccharin salts), cyclamate salts, acesulfam potassium (acesulfame K), and the free acid form of saccharin and aspartame based sweeteners such as L-aspartyl-phenylalanine methyl ester, Alitame® or Neotame®. The amount of sweetener or taste masking agents can vary with the desired amount of sweeteners or taste masking agents selected for a particular final composition.

Pharmaceutically acceptable binders in addition to those described above are also contemplated. Examples include cellulose derivatives including microcrystalline cellulose, low-substituted hydroxypropyl cellulose (e.g. LH 22, LH 21, LH 20, LH 32, LH 31, LH30); starches, including potato starch; croscarmellose sodium (i.e. cross-linked carboxymethylcellulose sodium salt; e.g. Ac-Di-Sol®); alginic acid or alginates; insoluble polyvinylpyrrolidone (e.g. Polyvidon® CL, Polyvidon® CL-M, Kollidon® CL, Polyplasdone® XL, Polyplasdone® XL-10); and sodium carboxymethyl starch (e.g. Primogel® and Explotab®).

Additional fillers, diluents or binders may be incorporated such as polyols, sucrose, sorbitol, mannitol, Erythritol®, Tagatose®, lactose (e.g., spray-dried lactose, α-lactose, β-lactose, Tabletose®, various grades of Pharmatose®, Microtose or Fast-Floc®), microcrystalline cellulose (e.g., various grades of Avicel®, such as Avicel® PH101, Avicel® PH102 or Avicel® PH105, Elcema® P100, Emcocel®, Vivacel®, Ming Tai® and Solka-Floc®), hydroxypropylcellulose, L-hydroxypropylcellulose (low-substituted) (e.g. L-HPC-CH31, L-HPC-LH11, LH 22, LH 21, LH 20, LH 32, LH 31, LH30), dextrins, maltodextrins (e.g. Lodex® 5 and Lodex® 10), starches or modified starches (including potato starch, maize starch and rice starch), sodium chloride, sodium phosphate, calcium sulfate, and calcium carbonate.

The compositions described herein can be formulated or adapted for administration to a subject via any route (e.g. any route approved by the Food and Drug Administration (FDA)). Exemplary methods are described in the FDA's CDER Data Standards Manual, version number 004 (which is available at fda.give/cder/dsm/DRG/drg00301.html).

Pharmaceutical compositions are typically formulated to be compatible with its intended route of administration. Examples of routes of administration include parenteral (subcutaneous, intracutaneous, intravenous, intradermal, intramuscular, intra-articular, intraarterial, intrasynovial, intrasternal, intrathecal, intralesional and intracranial injection or infusion techniques), oral (e.g., inhalation or through a feeding tube), transdermal (topical), transmucosal, and rectal administration.

Pharmaceutical compositions can be in the form of a solution or powder for inhalation and/or nasal administration. In some embodiments, the pharmaceutical composition is formulated as a powder filled sachet. Suitable powders may include those that are substantially soluble in water. Pharmaceutical compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions can be orally administered in any orally acceptable dosage form including, but not limited to, powders, capsules, tablets, emulsions and aqueous suspensions, dispersions and solutions. In the case of powders for oral administration, the powders can be substantially dissolved in water prior to administration. In the case of tablets for oral use, carriers which are commonly used include lactose and corn starch. Lubricating agents, such as magnesium stearate, may be added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, the compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

In some embodiments, therapeutic compositions disclosed herein can be formulated for sale in the US, imported into the US, and/or exported from the US. The pharmaceutical compositions can be included in a container, pack, or dispenser together with instructions for administration. In some embodiments, the invention provides kits that include the bile acid and phenylbutyrate compounds. The kit may also include instructions for the physician and/or patient, syringes, needles, box, bottles, vials, etc.

### III. Methods of Treatment

Applicant has discovered that a combination of a bile acid (e.g. TURSO) and a phenylbutyrate compound (e.g. sodium phenylbutyrate) can be used for treating one or more symptoms of ALS, and has surprisingly found that the combination inhibits one or more CYPs (e.g. CYP1A2, CYP2B6, CYP2C8, CYP2C9, CYP2C19, CYP2D6, and CYP3A (CYP3A4)) and transporters (e.g. Organic Anion Transporter 1 (OAT1)). When substrates of CYPs or transporters are administered concomitantly with a composition comprising a bile acid and a phenylbutyrate compound, the levels and/or effective dose of the substrates may be increased, which may result in an increase in any toxic effects associated with the substrates.

Accordingly, the present disclosure provides methods of treating at least one symptom of ALS in a subject who is receiving a substrate of CYP or a substrate of transporters, by adjusting the dosage of the substrate. Such adjustment may provide similar plasma concentrations of the substrate (or a metabolite thereof) as, and may be as effective as, the dosage of the substrate administered in the absence of the bile acid and the phenylbutyrate compound.

Applicant discloses herein methods for treating at least one symptom of ALS in a subject who has received a first dosage of a substrate of a CYP or a transporter, the method including administering to the subject a composition comprising about 1 gram of TURSO and about 3 grams of sodium phenylbutyrate, monitoring the subject for response to the substrate, and administering a second dosage of the substrate, wherein the second dosage is less than the first dosage. Monitoring the subject for response to the substrate can include determining the level of the substrate or a metabolite thereof in a biological sample from the subject, determining a blood INR level of the subject, or monitoring for known adverse events, overdose symptoms or side effects associated with the substrate.

In one aspect, provided herein are methods of treating at least one symptom of ALS in a subject, including (a) administering to a subject who has received a first dosage of a substrate of a cytochrome P450 (CYP) a composition comprising about 1 gram of TURSO and about 3 grams of sodium phenylbutyrate, (b) determining or having determined a first level of the substrate or a metabolite thereof in a first biological sample from the subject, and (c) administering to the subject a second dosage of the substrate, wherein the second dosage is lower than the first dosage.

The present disclosure also provides methods of treating at least one symptom of ALS in a subject, including (a) administering to a subject who has received a first dosage of warfarin a composition comprising about 1 gram of TURSO and about 3 grams of sodium phenylbutyrate; (b) determining or having determined a first blood INR level of the subject; and (c) administering to the subject a second dosage of warfarin, wherein the second dosage is lower than the first dosage.

Also provided are method of treating at least one symptom of ALS in a subject, including (a) administering to a subject who has received a first dosage of a substrate of Organic Anion Transporter 1 (OAT1) a composition comprising about 1 gram of TURSO and about 3 grams of sodium phenylbutyrate; (b) determining or having determined a first level of the substrate or a metabolite thereof in a first biological sample from the subject; and (c) administering to the subject a second dosage of the substrate, wherein the second dosage is lower than the first dosage.

The present disclosure further provides methods for treating at least one symptom of ALS in a subject who has received a first dosage of a narrow therapeutic index (NTI) drug, the method including administering to the subject a composition comprising about 1 gram of TURSO and about 3 grams of sodium phenylbutyrate, monitoring the subject for response to the NTI drug, and administering a second dosage of the NTI drug, wherein the second dosage is less than the first dosage. In one aspect, provided herein are methods of treating at least one symptom of ALS in a subject, including (a) administering to a subject who has received a first dosage of a narrow therapeutic index (NTI) drug a composition comprising about 1 gram of TURSO and about 3 grams of sodium phenylbutyrate; (b) determining or having determined a first level of the NTI drug or a metabolite thereof in a first biological sample from the subject; and (c) administering to the subject a second dosage of the NTI drug, wherein the second dosage is lower than the first dosage.

In some embodiments of any of the methods described herein, the first and/or second dosage of the substrate or the NTI drug can be a daily (once, twice, or three times daily), once every two days, three times a week, or a weekly dosage, or a dosage based on some other basis. The second dosage of the substrate or the NTI drug can be less than the first dosage by about 1% to about 95% (e.g., about 5%, 10%, 15%, 20%, 25%, 30%, 35%, 40%, 45%, 50%, 55%, 60%, 65%, 70%, 75%, 80%, 85%, or 90%). In some instances, the second dosage involves administering the same or similar amount per administration but is less frequent as compared to the first dosage.

In some embodiments of any of the methods described herein, the methods further include step (d), determining or having determined a second level of the substrate or the metabolite thereof, or the NTI drug or the metabolite thereof, in a second biological sample from the subject. In some embodiments, the second level is lower than the first level.

In some embodiments of any of the methods described herein, the first biological sample can be obtained from the subject about 1 hour to about 72 hours (e.g. about 2, 4, 6, 8, 10, 16, 24, 32, 48, or 56 hours) after administration of the composition comprising TURSO and phenylbutyrate. The second biological sample can be taken from the subject about 1 hour to about 72 hours (e.g. about 2, 4, 6, 8, 10, 16, 24, 32, 48, or 56 hours) after administration of the second dosage of the substrate or the NTI drug.

In some embodiments of any of the methods described herein, the first and/or second biological sample can be a serum, plasma, urine, or saliva sample. Methods of measuring the level of a substrate or a metabolite thereof or an NTI drug or a metabolite thereof in a biological sample are known in the art. For example, immunoassay or liquid chromatography/tandem mass spectrometry (LC/MS) can be used.

### Narrow Therapeutic Index Drugs

The therapeutic index (TI) is the range of doses at which a medication is effective without unacceptable adverse events. Narrow therapeutic index (NTI) drugs are drugs where small differences in dose or blood concentration may lead to serious therapeutic failures and/or adverse drug reactions that are life-threatening or result in persistent or significant disability or incapacity. Therefore, there is only a very small range of doses at which the drug produces a beneficial effect without causing severe and potentially fatal complications.

In animal studies, the TI can be calculated as the lethal dose of a drug for 50% of the population (LD₅₀) divided by the minimum effective dose for 50% of the population (ED₅₀), i.e. TI=LD₅₀/ED₅₀. In clinical practice, the TI is the range of doses at which a medication appeared to be effective in clinical trials for a median of participants without unacceptable adverse effects. It is generally considered that a drug has a good safety profile if its TI exceeds the value of 10.

Many NTI drugs are known in the art. The FDA defines a drug product as having a narrow therapeutic index when (a) there is less than a twofold difference in median lethal dose (LD₅₀) and median effective dose values (ED₅₀) or (b) there is less than a twofold difference in the minimum toxic concentrations (MTC) and minimum effective concentrations (MEC) in the blood and (c) safe and effective use of the drug requires careful titration and patient monitoring.

In one aspect, provided herein are methods of treating at least one symptom of ALS in a subject, including (a) administering to a subject who has received a first dosage of a narrow therapeutic index (NTI) drug an effective amount of a composition comprising about 1 gram of TURSO and about 3 grams of sodium phenylbutyrate; (b) determining or having determined a first level of the NTI drug or a metabolite thereof in a first biological sample from the subject; and (c) administering to the subject a second dosage of the NTI drug, wherein the second dosage is lower than the first dosage. The methods can further include step (d), determining or having determined a second level of the NTI drug or the metabolite thereof in a second biological sample from the subject. Exemplary NTI drugs include but are not limited to mexiletine, alfentanil, quinidine, cyclosporine, warfarin and digoxin.

In some embodiments, the NTI drug is digoxin. Digoxin is a cardiac glycoside used in the treatment of mild to moderate heart failure and for ventricular response rate control in chronic atrial fibrillation. The subject may have received a first dosage of digoxin at about 0.1 to about 0.6 mg/day (e.g. about 0.125 to about 0.5 mg/day, about 0.1-0.4 mg, or about 0.375-0.5 mg/day). The second dosage of digoxin can be less than the first dosage by about 0.1 to about 0.475 mg/day (e.g. about 0.2, 0.25, 0.3, 0.35, 0.4 or 0.45 mg/day).

Metabolites of digoxin are known in the art. Exemplary metabolites include dihydrodigoxin and digoxigenin bisdigitoxoside.

In addition to or instead of determining the first and/or second level of the NTI drug or a metabolite thereof in a biological sample from the subject, other methods of monitoring the subject's response to the first dosage of the NTI drug are also contemplated herein. For instance, known adverse events, side effects, or symptoms of overdose associated with the NTI can be monitored. For example, symptoms of digoxin overdose include nausea, vomiting, visual changes, and arrhythmia.

### Cytochrome P450 and Substrates

Cytochrome P450 (CYPs) are a superfamily of enzymes that contain heme as a cofactor and function as monooxygenases. CYP and CYP450 are used interchangeably herein. CYPs are primarily membrane-associated proteins located either in the inner membrane of mitochondria or in the endoplasmic reticulum of cells. These proteins are present in most tissues of the body, and play important roles in clearance of various compounds, hormone synthesis and breakdown (e.g. estrogen and testosterone synthesis and metabolism), cholesterol synthesis, and vitamin D metabolism. CYPs also function to metabolize potentially toxic compounds, including drugs and products of endogenous metabolism such as bilirubin, principally in the liver.

CYPs are the major enzymes involved in drug metabolism. Effects on CYP activity are a major source of adverse drug interactions, since changes in CYP enzyme activity may affect the metabolism and clearance of various drugs. For example, if one drug inhibits the CYP-mediated metabolism of another drug, the second drug may accumulate within the body to toxic levels.

Cytochrome P450 are encoded by CYP genes. Humans have 18 families of cytochrome P450 genes, including CYP1 (CYP1A1, CYP1A2, CYP1B1, CYP1D1P), CYP2 (CYP2A6, CYP2A7, CYP2A13, CYP2B6, CYP2C8, CYP2C9, CYP2C18, CYP2C19, CYP2D6, CYP2E1, CYP2F1, CYP2J2, CYP2R₁, CYP2S1, CYP2U1, CYP2W1), CYP3 (CYP3A4, CYP3A5, CYP3A7, CYP3A43, CYP3A51P, CYP3A52P, CYP3A54P, CYP3A137P), CYP4 (CYP4A11, CYP4A22, CYP4B1, CYP4F2, CYP4F3, CYP4F8, CYP4F11, CYP4F12, CYP4F22, CYP4V2, CYP4X1, CYP4Z1, CYP4F3A, CYP4F3B), CYP5 (CYP5A1), CYP7 (CYP7A1, CYP7B1), CYP8 (CYP8A1, CYP8B1), CYP11 (CYP11A1, CYP11B1, CYP11B2), CYP17 (CYP17A1), CYP19 (CYP19A1), CYP20 (CYP20A1), CYP21 (CYP21A2, CYP21A1P), CYP24 (CYP24A1), CYP26 (CYP26A1, CYP26B1, CYP26C1), CYP27 (CYP27A1, CYP27B1, CYP27C1), CYP39 (CYP39A1), CYP46 (CYP46A1, CYP46A4P), and CYP51 (CYP51A1, CYP51P1, CYP51P2, CYP51P3).

The combination of a bile acid and a phenylbutyrate compound may inhibit one or more CYPs (e.g. any of the CYPs described herein or known in the art). For example, the combination of TURSO and sodium phenylbutyrate may inhibit one or more of CYP1A2, CYP2B6, CYP2C8, CYP2C9, CYP2C19, CYP2D6, or CYP3A (e.g. CYP3A4). The combination of TURSO and sodium phenylbutyrate may be a strong inhibitor of a CYP or a moderate inhibitor of a CYP. According to the FDA definition, strong CYP inhibitors are expected to increase the AUC of sensitive index substrates of the CYP by 5 fold or more. Moderate CYP inhibitors are expected to increase the AUC of sensitive index substrates of the CYP by between 2 fold and 5 fold, inclusive. Examples of clinical sensitive or moderate sensitive index substrates for exemplary CYPs are shown below.

| | Sensitive or moderate |
|---|---|
| CYP | sensitive index substrate |
| CYP1A2 | caffeine, tizanidine |
| CYP2C8 | repaglinide |
| CYP2C9 | tolbutamide, S-warfarin |
| | (moderate sensitive substrates) |
| CYP2C19 | lansoprazole, omeprazole |
| CYP2D6 | desipramine, |
| | dextromethorphan, nebivolol |
| CYP3A | midazolam, triazolam |

Index substrates predictably exhibit exposure increase due to inhibition or induction of a given metabolic pathway and are commonly used in prospective clinical DDI studies. Sensitive index substrates are index drugs that demonstrate an increase in AUC of ≥5-fold with strong index inhibitors of a given metabolic pathway in clinical DDI studies. Moderate sensitive substrates are drug that demonstrate an increase in AUC of ≥2 to <5-fold with strong index inhibitors of a given metabolic pathway in clinical DDI studies. Exemplary strong index inhibitors of CYPs are known in the art (see, https://www.fda.gov/drugs/drug-interactions-labeling/drug-development-and-drug-interactions-table-substrates-inhibitors-and-inducers).

A composition comprising TURSO and sodium phenylbutyrate, when administered to a subject concomitantly with a substrate of a CYP (e.g. CYP2B6, CYP2C8, CYP2C9, CYP2C19, CYP2D6, or CYP3A (e.g. CYP3A4)), may result in an increase in the level (e.g. plasma concentration) of the substrate in the subject. In some instances, a metabolite of TURSO or sodium phenylbutyrate is inhibitory to one of more CYPs. For example, a metabolite of phenylbutyrate, phenylacetylacetate, may be inhibitory to CYP1A2 and/or CYP2D6.

Substrates of CYPs can be a drug or other substances (e.g., xenobiotics) that are metabolized by a CYP. Substrates of a CYP can be pharmacologically active drugs that require metabolism to inactive form for clearance from the body of a subject, or metabolically activated drugs (e.g., prodrugs) that require conversion to active drug.

Many substrates of CYPs are known in the art. Exemplary substrates of CYP1A2 include alosetron, caffeine, duloxetine, melatonin, ramelteon, tasimelteon, tizanidine, clozapine, pirfenidone, ramosetron, and theophylline. Exemplary substrates of CYP2B6 include bupropion and efavirenz. Exemplary substrates of CYP2C8 include repaglinide, montelukast, pioglitazone, and rosiglitazone. Exemplary substrates of CYP2C9 include celecoxib, glimepiride, phenytoin, warfarin, tolbutamide, and warfarin. Exemplary substrates of CYP2C19 include S-mephenytoin, omeprazole, diazepam, lansoprazole, rabeprazole, or voriconazole. Exemplary substrates of CYP2D6 include atomoxetine, desipramine, dextromethorphan, eliglustat, nebivolol, nortriptyline, perphenazine, tolterodine, R-venlafaxine, encainide, imipramine, metoprolol, propafenone, propranolol, tramadol, trimipramine, and S-venlafaxine. Exemplary substrates of CYP3A include alfentanil, avanafil, buspirone, conivaptan, cyclosporine, quinidine, darifenacin, darunavir, ebastine, everolimus, ibrutinib, lomitapide, lovastatin, midazolam, naloxegol, nisoldipine, saquinavir, simvastatin, sirolimus, tacrolimus, tipranavir, triazolam, vardenafil, budesonide, dasatinib, dronedarone, eletriptan, eplerenone, felodipine, indinavir, lurasidone, maraviroc, quetiapine, sildenafil, ticagrelor, tolvaptan, alprazolam, aprepitant, atorvastatin, colchicine, eliglustat, pimozide, rilpivirine, rivaroxaban, or tadalafil. Exemplary substrates of CYP3A4 include alfentanil, cyclosporine, and quinidine.

Methods of identifying substrates of CYPs are known in the art. For example, cytochrome P450 reaction-phenotyping can be used (see, Zhang et al. Expert Opin Drug Metab Toxicol. 2007 Oct; 3(5):667-87).

Provided herein are methods of treating at least one symptom of ALS in a subject, the method including (a) administering to a subject who has received a first dosage of a substrate of a CYP an effective amount of a composition comprising about 1 gram of TURSO and about 3 grams of sodium phenylbutyrate, (b) determining or having determined a first level of the substrate or a metabolite thereof in a first biological sample from the subject, and (c) administering to the subject a second dosage of the substrate, wherein the second dosage is lower than the first dosage. The method can further include step (d), determining or having determined a second level of the substrate or a metabolite thereof in a second biological sample from the subject. In some embodiments, the second level of the substrate is lower than the first level.

In some embodiments of any of the methods described herein, the CYP is CYP1A2, CYP2B6, CYP2C8, CYP2C9, CYP2C19, CYP2D6, or CYP3A. The substrate of a CYP can be a substrate of CYP1A2, for example, mexiletine. The substrate of a CYP can be a substrate of CYP3A4, for example, alfentanil, cyclosporine, and quinidine. The substrate of CYP can be a substrate of CYP2C9, for example, warfarin. The substrate of a CYP can be a substrate of CYP2D6, for example mexiletine.

The substrates of CYP can also be an NTI drug. For example, mexiletine, alfentanil, quinidine, cyclosporine, warfarin are substrates of CYP that have a narrow therapeutic index.

In some embodiments, the substrate of a CYP is mexiletine. Mexiletine can be used for the treatment of ventricular arrhythmias, organ transplant rejection, myotonia, renal impairment, or hepatic impairment. Mexiletine can be metabolized by CYP2D6 and CYP1A2. The subject may have received a first dosage of mexiletine at about 100 to about 1200 mg/day (e.g. about 200 to about 1000 mg/day, about 250 to about 950 mg/day, about 300 to about 900 mg/day, about 300 mg/day, or about 900 mg/day). The second dosage of mexiletine can be less than the first dosage by about 10 to about 1150 mg/day (e.g. about 50, 100, 150, 200, 250, 300, 350, 400, 450, 500, 550, 600, 650, 700, 750, 800, 900, 1000, or 1100 mg/day).

In some embodiments, the substrate of a CYP is alfentanil. Alfentanil is a short-acting opioid anesthetic and analgesic derivative of fentanyl. Alfentanil can be used as an anesthetic during surgery, for supplementation of analgesia during surgical procedures, and as an analgesic for critically ill patients. Alfentanil can be metabolized by CYP3A4 and CYP3A5. The first dosage of alfentanil may be dependent on the length of the anesthesia. Exemplary first dosages (e.g. either the induction or the maintenance dosage) of alfentanil are shown below.

| Use | Induction | Maintenance |
|---|---|---|
| Incremental injection: | 8-20 mcg/kg IV | 3-5 mcg/kg IV |
| Anesthesia <30 Minutes | | increments |
| | | q5-20 min, or 0.5-1 |
| | | mcg/kg/min IV |
| Incremental Injection: | 20-50 mcg/kg IV | 5-15 mcg/kg IV |
| Anesthesia 30-60 | | increments |
| Minutes | | q5-20 min |
| Anesthetic Induction: | 130-245 mcg/kg IV | 0.5-1.5 mcg/kg/min IV |
| Anesthesia >45 Minutes | | |
| Continuous Infusion: For | 50-75 mcg/kg IV | 0.5-3 mcg/kg/min IV |
| Anesthesia >45 Minutes | | |

In some embodiments, the substrate of a CYP is quinidine. Quinidine is a stereoisomer of quinine. Quinidine can be used to restore normal sinus rhythm, treat atrial fibrillation and flutter, and treat ventricular arrhythmias. Quinidine can be metabolized by CYP3A4. For an oral dosage form, the first dosage of quinidine may be about 200 to about 650 mg every three or four times a day, or about 300 to 660 mg every eight to twelve hours, for example, where the subject is an adult. The second dosage of quinidine can be less than the first dosage by about 10 mg to about 600 mg per administration (e.g. by about 20, 40, 60, 80, 100, 120, 140, 160, 180, 200, 220, 240, 260, 280, 300, 350, 400, 450, 500, or 550 mg per administration). The second dosage of quinidine can also be less frequent than the first dosage of quinidine (e.g. administered for fewer times a day). For injection dosage form, the first dosage of quinidine may be about 190 to about 380 mg injected into the muscle every two to four hours, or up to 0.25 mg/kg of body weight per minute in a solution injected into a vein. The second dosage of quinidine can be less than the first dosage by about 10 mg to about 350 mg per administration (e.g. by about 20, 40, 60, 80, 100, 120, 140, 160, 180, 200, 220, 240, 260, 280, or 300 mg per administration). Where the subject is a child, the first dosage of quinidine may be about 30 to about 40 mg/kg of body weight per day, for an oral dosage form.

In some embodiments, the substrate of a CYP is cyclosporine. Cyclosporine can be metabolized by CYP3A4 and CYP3A5. Cyclosporine can be used for the prophylaxis of organ rejection in allogeneic kidney, liver, and heart transplants, or to prevent bone marrow transplant rejection. Cyclosporine is used for the treatment of patients with severe active rheumatoid arthritis (RA), or severe, recalcitrant, plaque psoriasis. The ophthalmic solution of cyclosporine is indicated to increase tear production in patients suffering from keratoconjunctivitis sicca. In addition, cyclosporine is approved for the treatment of steroid dependent and steroid-resistant nephrotic syndrome due to glomerular diseases which may include minimal change nephropathy, focal and segmental glomerulosclerosis or membranous glomerulonephritis. Cyclosporine is also commonly used for the treatment of various autoimmune and inflammatory conditions such as atopic dermatitis, blistering disorders, ulcerative colitis, juvenile rheumatoid arthritis, uveitis, connective tissue diseases, as well as idiopathic thrombocytopenic purpura. The subject may have received a first dosage of cyclosporine at about 0.5 to about 15 mg/kg/day of body weight (e.g., about 0.5 to about 5 mg/kg/day, about 1 to about 4 mg/kg/day, about 2.5 mg/kg/day, or about 12 to about 15 mg/kg/day). The second dosage of cyclosporine can be less than the first dosage by about 0.1 to about 14 mg/kg/day (e.g., about 0.5 to about 2.5 mg/kg/day, about 1 to about 5 mg/kg/day).

In some embodiments, the substrate of a CYP is warfarin. Warfarin can be used as an anticoagulant to prevent blood clots (e.g. deep vein thrombosis and pulmonary embolism), and to prevent stroke in subjects who have atrial fibrillation, valvular heart disease or artificial heart valves. Warfarin can be metabolized by CYP2C9, CYP1A2 and CYP3A. The subject may have received a first dosage of warfarin at about 0.5 to about 12 mg/day (e.g., about 2 to about 6 mg/day, about 5 to about 8 mg/day, or about 7 to about 10 mg/day). The second dosage of warfarin can be less than the first dosage of warfarin for about 0.5 to about 9.5 mg/day (e.g. about 0.5, 1, 1.5, 2, 2.5, 3, 3.5, 4, 4.5, 5, 5.5, 6, 6.5, 7, 7.5, 8, 8.5, or 9 mg/day).

Metabolites of the CYP substrates described herein are known in the art. For example, metabolites of mexiletine include but are not limited to, p-hydroxymexiletine, hydroxy-methylmexiletine, N-hydroxy-mexiletine, and N-methylmexiletine. Exemplary metabolites of alfentanil include noralfentanil and N-phenylpropionamide. Exemplary metabolites of quinidine include 3-hydroxyquinidine, 2′-Oxoquinidinone, O-desmethylquinidine, and quinidine-N-oxide. Exemplary metabolites of cyclosporine include M1, M9, and M4N. Additional examples of cyclosporine metabolites can be found in Yatscoff et al. Clin Biochem, Vol 24, pp. 23-35, 1991. Exemplary metabolites of R-warfarin include hydroxywarfarin, 10-hydroxywarfarin, diastereoisomeric alcohols. S-warfarin can be metabolized to 7-hydroxywarfarin.

In addition to or instead of determining the first and/or second level of the substrate or a metabolite thereof in a biological sample from the subject, other methods of monitoring the subject's response to the first dosage of the substrate are also contemplated herein. For instance, known adverse events, side effects, or symptoms of overdose associated with the substrate can be monitored. Symptoms of mexiletine overdose can include nausea, hypotension, sinus bradycardia, paresthesia, seizures, bundle branch block, AV heart block, asystole, ventricular tachyarrythmia, including ventricular fibrillation, cardiovascular collapse, or coma. Symptoms of alfentanil overdose can include characteristic rigidity of the skeletal muscles, cardiac and respiratory depression, and narrowing of the pupils. Symptoms of quinidine overdose can include irregular heartbeat, diarrhea, vomiting, headache, ringing in the ears or loss of hearing, vision changes (blurred vision or light sensitivity), or confusion. Cyclosporine overdose symptoms can include hepatotoxicity and nephrotoxicity. Warfarin overdose symptoms mainly involve bleeding (e.g., appearance of blood in stools or urine, hematuria, excessive menstrual bleeding, melena, petechiae, excessive bruising or persistent oozing from superficial injuries, unexplained fall in hemoglobin) which is a manifestation of excessive anticoagulation.

Where the substrate of CYP is warfarin, the subject's response can also be monitored through prothrombin time-international normalized ratio (PT-INR). PT-INR measures how much time it takes for a subject's blood to form a clot and can be used to determine if the appropriate dose of warfarin was administered. Methods of monitoring a subject's INR levels are known in the art. A typical INR target ranges from 2.0 to 3.5 (e.g., 2.0 to 3.3, 2.0 to 3.0, 2.0 to 2.8, 2.0 to 2.6, 2.0 to 2.4, 2.0 to 2.2, 2.2 to 3.5, 2.2 to 3.3, 2.2 to 3.0, 2.2 to 2.8, 2.2 to 2.6, 2.2 to 2.4, 2.4 to 3.5, 2.4 to 3.3, 2.4 to 3.0, 2.4 to 2.8, 2.4 to 2.6, 2.6 to 3.5, 2.6 to 3.3, 2.6 to 3.0, 2.6 to 2.8, 2.8 to 3.5, 2.8 to 3.3, 2.8 to 3.0, 3.0 to 3.5, 3.0 to 3.3, or 3.3 to 3.5). INR can be monitored once every day, twice a week, once a week, once every two weeks, once every three weeks, or once every four weeks.

Warfarin dosage can be adjusted to bring the PT-INR blood test into the target range. INR can be monitored more often when the dose is being changed, when the subject starts or stops another medication, or when the subject's medical condition changes. It can be monitored less often when the dose is stable. A typical frequency of monitoring for stable dosing is approximately every four or six weeks (e.g., every four weeks, every five weeks, or every six weeks).

In one aspect, provided herein are methods of treating at least one symptom of ALS in a subject, the method including (a) administering to a subject who has received a first dosage of warfarin an effective amount of a composition comprising about 1 gram of TURSO and about 3 grams of sodium phenylbutyrate; (b) determining or having determined a first blood INR level of the subject; and (c) administering to the subject a second dosage of warfarin, wherein the second dosage is lower than the first dosage.

The first dosage of warfarin can be about 0.5 to about 12 mg/day (e.g., about 2 to about 6 mg/day, about 5 to about 8 mg/day, or about 7 to about 10 mg/day). The second dosage of warfarin can be less than the first dosage of warfarin for about 0.5 to about 9.5 mg/day (e.g. about 0.5, 1, 1.5, 2, 2.5, 3, 3.5, 4, 4.5, 5, 5.5, 6, 6.5, 7, 7.5, 8, 8.5, or 9 mg/day).

In some embodiments, step (b) includes determining or having determined the first blood INR level of the subject once daily. The first blood INR level of the subject can be determined at any suitable frequency, e.g. once every other day, twice a week, once a week, once every two weeks, once every three weeks, or once every four weeks. In some embodiments, the first blood INR level is about 3.0 or higher (e.g., about 3.5, about 4.0, about 4.5, about 5.0, about 5.5, about 6.0, about 6.5, about 7.0, about 7.5, about 8.0, about 8.5, about 9.0, about 9.5, or about 10.0). In some embodiments, the first blood INR level is about 4.0 or higher (e.g., about 4.5, about 5.0, about 5.5, about 6.0, about 6.5, about 7.0, about 7.5, about 8.0, about 8.5, about 9.0, about 9.5, or about 10.0). In some embodiments of any of the methods described herein, the methods further include step (d), determining or having determined a second blood INR level of the subject. In some embodiments, the second blood INR level is lower than the first blood INR level.

### Transporters

Transporters are membrane proteins involved in the uptake or efflux of drugs, and are present in various tissues such as the lymphocytes, intestine, liver, kidney, placenta and central nervous system. Transporters can have a significant impact on the pharmacokinetics of endogenous (e.g., ions, vitamins, or amino acids) and exogenous compounds. Co-administered drugs or nutrients can influence transporter activity which may lead to changes in the pharmacokinetics of drugs and, as a result, possibly lead to reduced efficacy or increased toxicity (e.g., drug-drug or drug-nutrient interactions). Exemplary transporters include P-glycoprotein (P-gp), breast cancer resistance protein (BCRP), organic anion transporting polypeptides 1B1 and 1B3 (OATP1B1 and OATP1B3), organic anion transporters 1 and 3 (OAT1 and OAT3), organic cation transporters 1 and 2 (OCT1 and OCT2), and multidrug and toxin extrusion proteins 1 and 2K (MATE1 and MATE2K).

Transporters can be divided into (i) efflux transporters belonging to the ATP-binding cassette (ABC) family and (ii) uptake transporters belonging to the solute carrier (SLC) family that mediate the influx or bidirectional movement across the cell membrane. Uptake transporters at the blood brain barrier (BBB) are responsible for bringing solutes from circulation into the endothelial cells (i.e., apical/luminal membrane) and then into the brain across the basolateral membrane. Exemplary uptake transporters include H+/ditripeptide transporter, organic anion transporting polypeptide (OATPs, e.g., OATP1B1 or OATP1B3), organic anion transporter (e.g., OAT1 or OAT3), and organic cation transporter (OCT) 1 and OCT2.

Efflux transporters pump compounds back into the blood as they traverse the apical cell membrane (i.e., the blood side) and also pump compounds out of the cell into the brain on the basolateral side. Exemplary efflux transporters include P-glycoprotein (P-gp, ABCB1), multidrug resistance-associated protein (MRP) 1 and MRP2, and breast cancer resistance protein (BCRP, ABCG2).

Applicant has discovered that the combination of a bile acid or a pharmaceutically acceptable salt thereof and a phenylbutyrate compound can inhibit one or more transporters (e.g. any of the transporters described herein or known in the art). For example, the combination of TURSO and sodium phenylbutyrate can inhibit one or more transporters (e.g. OAT1). Accordingly, Applicant discloses herein methods for treating at least one symptom of ALS in a subject who has received a first dosage of a substrate of a transporter, the method including administering to the subject a composition comprising about 1 gram of TURSO and about 3 grams of sodium phenylbutyrate, monitoring the subject for response to the substrate, and administering a second dosage of the substrate, wherein the second dosage is less than the first dosage. Monitoring the subject for response to the substrate can include determining or having determined the level of the substrate or a metabolite thereof in a biological sample from the subject, or monitoring for known adverse events, overdose symptoms or side effects associated with the substrate.

Substrates of transporters are known in the art. Exemplary substrate of P-gp include digoxin, fexofenadine, loperamide, quinidine, talinolol, and vinblastine. Exemplary substrate of BCRP include 2-amino-1-methyl-6-phenylimidazo[4,5-b]pyridine (PhIP), coumestrol, daidzein, dantrolene, estrone-3-sulfate, genistein, prazosin, and sulfasalazine. Exemplary substrates of OATP1B1 or OATP1B3 include cholecystokinin octapeptide (CCK-8), estradiol-17β-glucuronide, estrone-3-sulfate, pitavastatin, pravastatin, telmisartan, and rosuvastatin. Exemplary substrates of OAT1 include penicillin, non-steroidal anti-inflammatory drug (NSAID) (e.g. diclofenac, ketoprofen, or methotrexate), HIV protease inhibitor, and antiviral drug (e.g., adefovir, cidofovir, and tenofovir). Exemplary substrates of OAT3 include benzylpenicillin, estrone-3-sulfate, methotrexate, and pravastatin. Exemplary substrates of MATE1 or MATE-2K include metformin, 1-methyl-4-phenylpyridinium (MPP+), and tetraethylammonium (TEA). Exemplary substrates of OCT2 include metformin, 1-methyl-4-phenylpyridinium (MPP+), and tetraethylammonium.

In one aspect, provided herein are methods of treating at least one symptom of ALS in a subject, the method including (a) administering to a subject who has received a first dosage of a substrate of Organic Anion Transporter 1 (OAT1) an effective amount of a composition comprising about 1 gram of TURSO and about 3 grams of sodium phenylbutyrate; (b) determining or having determined a first level of the substrate in a first biological sample from the subject; and (c) administering to the subject a second dosage of the substrate, wherein the second dosage is lower than the first dosage. The method can further include step (d), determining or having determined a second level of the substrate in a second biological sample from the subject. In some embodiments, the second level of the substrate is lower than the first level.

Exemplary substrates of OAT1 include penicillin, non-steroidal anti-inflammatory drugs (NSAID) (e.g. diclofenac, ketoprofen, or methotrexate), HIV protease inhibitors, and antiviral drugs (e.g. Adefovir, Cidofovir, or Tenofovir).

### Administration of TURSO and Sodium Phenylbutyrate

The methods described herein include administering to the subject a bile acid or pharmaceutically acceptable salt thereof, and a phenylbutyrate compound. The bile acid or a pharmaceutically acceptable salt thereof and the phenylbutyrate compound can be administered separately or concurrently, including as a part of a regimen of treatment. The compounds can be administered daily (e.g. once a day, twice a day, or three times a day or more), weekly, monthly, or quarterly. The compounds can be administered over a period of weeks, months, or years. For example, the compounds can be administered over a period of at least or about 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, or at least or about 5 years, or more. The compounds can be administered once a day or twice a day for 60 days or less (e.g., 55 days, 50 days, 45 days, 40 days, 35 days, 30 days or less). Alternatively, the bile acid and phenylbutyrate compound can be administered once a day or twice a day for more than 60 days (e.g., more than 65, 70, 75, 80, 85, 90, 95, 100, 105, 110, 115, 120, 130, 140, 150, 160, 180, 200, 250, 300, 400, 500, 600 days).

In some embodiments, the methods provided herein include administering an effective amount of a composition comprising about 1 gram of TURSO and about 3 grams of sodium phenylbutyrate. TURSO can be administered at an amount of about 0.5 to about 5 grams per day (e.g., about 0.5 to about 4.5, about 0.5 to about 4, about 0.5 to about 3.5, about 0.5 to about 3, about 0.5 to about 2.5, about 0.5 to about 2, about 0.5 to about 1.5, about 0.5 to about 1, about 1 to about 5, about 1 to about 4.5, about 1 to about 4, about 1 to about 3.5, about 1 to about 3, about 1 to about 2.5, about 1 to about 2, about 1 to about 1.5, about 1.5 to about 5, about 1.5 to about 4.5, about 1.5 to about 4, about 1.5 to about 3.5, about 1.5 to about 3, about 1.5 to about 2.5, about 1.5 to about 2, about 2 to about 5, about 2 to about 4.5, about 2 to about 4, about 2 to about 3.5, about 2 to about 3, about 2 to about 2.5, about 2.5 to about 5, about 2.5 to about 4.5, about 2.5 to about 4, about 2.5 to about 3.5, about 2.5 to about 3, about 3 to about 5, about 3 to about 4.5, about 3 to about 4, about 3 to about 3.5, about 3.5 to about 5 about 3.5 to about 4.5, about 3.5 to about 4, about 4 to about 5, about 4 to about 4.5, or about 4.5 to about 5 grams). In some embodiments, TURSO is administered at an amount of about 1 to about 2 grams per day, inclusive (e.g., about 1 to about 1.8 grams, about 1 to about 1.6 grams, about 1 to about 1.4 grams, about 1 to about 1.2 grams, about 1.2 to about 2.0 grams, about 1.2 to about 1.8 grams, about 1.2 to about 1.6 grams, about 1.2 to about 1.4 grams, about 1.4 to about 2.0 grams, about 1.4 to about 1.8 grams, about 1.4 to about 1.6 grams, about 1.6 to about 2.0 grams, about 1.6 to about 1.8 grams, about 1.8 to about 2.0 grams). In some embodiments, TURSO is administered at an amount of about 1 gram per day. In some embodiments, TURSO is administered at an amount of about 2 grams per day. For example, TURSO can be administered at an amount of about 1 gram twice a day.

Sodium phenylbutyrate can be administered at an amount of about 0.5 to about 10 grams per day (e.g., about 1 to about 10, about 1 to about 9, about 1 to about 8, about 1 to about 7, about 1 to about 6, about 1 to about 5, about 1 to about 4, about 1 to about 3, about 1 to about 2, about 2 to about 10, about 2 to about 9, about 2 to about 8, about 2 to about 7, about 2 to about 6, about 2 to about 5, about 2 to about 4, about 2.5 to about 9.5, about 2.5 to about 8.5, about 2.5 to about 7.5, about 2.5 to about 6.5, about 2.5 to about 5.5, about 2.5 to about 4.5, about 3 to about 10, about 3 to about 9, about 3 to about 8, about 3 to about 7, about 3 to about 6.5, about 3 to about 6, about 3 to about 5, about 4 to about 10, about 4 to about 9, about 4 to about 8, about 4 to about 7, about 4 to about 6, about 5 to about 10, about 5 to about 9, about 5 to about 8, about 5 to about 7, about 6 to about 10, about 6 to about 9, about 6 to about 8, about 7 to about 10, about 7 to about 9, about 8 to about 10 grams per day). In some embodiments, sodium phenylbutyrate is administered at an amount of about 3 to about 6 grams per day, inclusive (e.g., about 3 to about 5.5 grams, about 3 to about 5.0 grams, about 3 to about 4.5 grams, about 3 to about 4.0 grams, about 3 to about 3.5 grams, about 3.5 to about 6 grams, about 3.5 to about 5.5 grams, about 3.5 to about 5.0 grams, about 3.5 to about 4.5 grams, about 3.5 to about 4.0 grams, about 4.0 to about 6 grams, about 4.0 to about 5.5 grams, about 4.0 to about 5.0 grams, about 4.0 to about 4.5 grams, about 4.5 to about 6 grams, about 4.5 to about 5.5 grams, about 4.5 to about 5.0 grams, about 5.0 to about 6 grams, about 5.0 to about 5.5 grams, or about 5.5 to about 6.0 grams). In some embodiments, sodium phenylbutyrate is administered at an amount of about 3 grams per day. In some embodiments, sodium phenylbutyrate is administered at an amount of about 6 grams per day. For example, sodium phenylbutyrate can be administered at an amount of about 3 grams twice a day. In some embodiments, the bile acid and phenylbutyrate compound are administered at a ratio by weight of about 2.5:1 to about 3.5:1 (e.g., about 3:1).

The methods described herein can include administering about 1 gram of TURSO once a day and about 3 grams of sodium phenylbutyrate once a day, or about 1 gram of TURSO twice a day and about 3 grams of sodium phenylbutyrate twice a day. The methods can include administering about 1 gram of TURSO once a day and about 3 grams of sodium phenylbutyrate once a day for at least 14 days (e.g., at least 15, 16, 17, 18, 19, 20, 21, 22, 23, 24, 27, 30, 35, or 40 days), followed by administering about 1 gram of TURSO twice a day and about 3 grams of sodium phenylbutyrate twice a day or at least a day (e.g. at least 30, 40, 50, 60, 80, 100, 120, 150, 180, 250, 300, or 400 days). For example, the methods can include administering about 1 gram of TURSO once a day and about 3 grams of sodium phenylbutyrate once a day for 14-21 days, followed by administering about 1 gram of TURSO twice a day and about 3 grams of sodium phenylbutyrate twice a day.

In some embodiments, the methods described herein include administering to a subject about 10 mg/kg to about 50 mg/kg of body weight of TURSO per day (e.g., about 10 mg/kg to about 48 mg/kg, about 10 mg/kg to about 46 mg/kg, about 10 mg/kg to about 44 mg/kg, about 10 mg/kg to about 42 mg/kg, about 10 mg/kg to about 40 mg/kg, about 10 mg/kg to about 38 mg/kg, about 10 mg/kg to about 36 mg/kg, about 10 mg/kg to about 34 mg/kg, about 10 mg/kg to about 32 mg/kg, about 10 mg/kg to about 30 mg/kg, about 10 mg/kg to about 28 mg/kg, about 10 mg/kg to about 26 mg/kg, about 10 mg/kg to about 24 mg/kg, about 10 mg/kg to about 22 mg/kg, about 10 mg/kg to about 20 mg/kg, about 10 mg/kg to about 18 mg/kg, about 10 mg/kg to about 16 mg/kg, about 10 mg/kg to about 14 mg/kg, about 10 mg/kg to about 12 mg/kg, about 12 mg/kg to about 50 mg/kg, about 12 mg/kg to about 48 mg/kg, about 12 mg/kg to about 46 mg/kg, about 12 mg/kg to about 44 mg/kg, about 12 mg/kg to about 42 mg/kg, about 12 mg/kg to about 40 mg/kg, about 12 mg/kg to about 38 mg/kg, about 12 mg/kg to about 36 mg/kg, about 12 mg/kg to about 34 mg/kg, about 12 mg/kg to about 32 mg/kg, about 12 mg/kg to about 30 mg/kg, about 12 mg/kg to about 28 mg/kg, about 12 mg/kg to about 26 mg/kg, about 12 mg/kg to about 24 mg/kg, about 12 mg/kg to about 22 mg/kg, about 12 mg/kg to about 20 mg/kg, about 12 mg/kg to about 18 mg/kg, about 12 mg/kg to about 16 mg/kg, about 12 mg/kg to about 14 mg/kg, about 14 mg/kg to about 50 mg/kg, about 14 mg/kg to about 48 mg/kg, about 14 mg/kg to about 46 mg/kg, about 14 mg/kg to about 44 mg/kg, about 14 mg/kg to about 42 mg/kg, about 14 mg/kg to about 40 mg/kg, about 14 mg/kg to about 38 mg/kg, about 14 mg/kg to about 36 mg/kg, about 14 mg/kg to about 34 mg/kg, about 14 mg/kg to about 32 mg/kg, about 14 mg/kg to about 30 mg/kg, about 14 mg/kg to about 28 mg/kg, about 14 mg/kg to about 26 mg/kg, about 14 mg/kg to about 24 mg/kg, about 14 mg/kg to about 22 mg/kg, about 14 mg/kg to about 20 mg/kg, about 14 mg/kg to about 18 mg/kg, about 14 mg/kg to about 16 mg/kg, about 16 mg/kg to about 50 mg/kg, about 16 mg/kg to about 48 mg/kg, about 16 mg/kg to about 46 mg/kg, about 16 mg/kg to about 44 mg/kg, about 16 mg/kg to about 42 mg/kg, about 16 mg/kg to about 40 mg/kg, about 16 mg/kg to about 38 mg/kg, about 16 mg/kg to about 36 mg/kg, about 16 mg/kg to about 34 mg/kg, about 16 mg/kg to about 32 mg/kg, about 16 mg/kg to about 30 mg/kg, about 16 mg/kg to about 28 mg/kg, about 16 mg/kg to about 26 mg/kg, about 16 mg/kg to about 24 mg/kg, about 16 mg/kg to about 22 mg/kg, about 16 mg/kg to about 20 mg/kg, about 16 mg/kg to about 18 mg/kg, about 18 mg/kg to about 50 mg/kg, about 18 mg/kg to about 48 mg/kg, about 18 mg/kg to about 46 mg/kg, about 18 mg/kg to about 44 mg/kg, about 18 mg/kg to about 42 mg/kg, about 18 mg/kg to about 40 mg/kg, about 18 mg/kg to about 38 mg/kg, about 18 mg/kg to about 36 mg/kg, about 18 mg/kg to about 34 mg/kg, about 18 mg/kg to about 32 mg/kg, about 18 mg/kg to about 30 mg/kg, about 18 mg/kg to about 28 mg/kg, about 18 mg/kg to about 26 mg/kg, about 18 mg/kg to about 24 mg/kg, about 18 mg/kg to about 22 mg/kg, about 18 mg/kg to about 20 mg/kg, about 20 mg/kg to about 50 mg/kg, about 20 mg/kg to about 48 mg/kg, about 20 mg/kg to about 46 mg/kg, about 20 mg/kg to about 44 mg/kg, about 20 mg/kg to about 42 mg/kg, about 20 mg/kg to about 40 mg/kg, about 20 mg/kg to about 38 mg/kg, about 20 mg/kg to about 36 mg/kg, about 20 mg/kg to about 34 mg/kg, about 20 mg/kg to about 32 mg/kg, about 20 mg/kg to about 30 mg/kg, about 20 mg/kg to about 28 mg/kg, about 20 mg/kg to about 26 mg/kg, about 20 mg/kg to about 24 mg/kg, about 20 mg/kg to about 22 mg/kg, about 22 mg/kg to about 50 mg/kg, about 22 mg/kg to about 48 mg/kg, about 22 mg/kg to about 46 mg/kg, about 22 mg/kg to about 44 mg/kg, about 22 mg/kg to about 42 mg/kg, about 22 mg/kg to about 40 mg/kg, about 22 mg/kg to about 38 mg/kg, about 22 mg/kg to about 36 mg/kg, about 22 mg/kg to about 34 mg/kg, about 22 mg/kg to about 32 mg/kg, about 22 mg/kg to about 30 mg/kg, about 22 mg/kg to about 28 mg/kg, about 22 mg/kg to about 26 mg/kg, about 22 mg/kg to about 24 mg/kg, about 24 mg/kg to about 50 mg/kg, about 24 mg/kg to about 48 mg/kg, about 24 mg/kg to about 46 mg/kg, about 24 mg/kg to about 44 mg/kg, about 24 mg/kg to about 42 mg/kg, about 24 mg/kg to about 40 mg/kg, about 24 mg/kg to about 38 mg/kg, about 24 mg/kg to about 36 mg/kg, about 24 mg/kg to about 34 mg/kg, about 24 mg/kg to about 32 mg/kg, about 24 mg/kg to about 30 mg/kg, about 24 mg/kg to about 28 mg/kg, about 24 mg/kg to about 26 mg/kg, about 26 mg/kg to about 50 mg/kg, about 26 mg/kg to about 48 mg/kg, about 26 mg/kg to about 46 mg/kg, about 26 mg/kg to about 44 mg/kg, about 26 mg/kg to about 42 mg/kg, about 26 mg/kg to about 40 mg/kg, about 26 mg/kg to about 38 mg/kg, about 26 mg/kg to about 36 mg/kg, about 26 mg/kg to about 34 mg/kg, about 26 mg/kg to about 32 mg/kg, about 26 mg/kg to about 30 mg/kg, about 26 mg/kg to about 28 mg/kg, about 28 mg/kg to about 50 mg/kg, about 28 mg/kg to about 48 mg/kg, about 28 mg/kg to about 46 mg/kg, about 28 mg/kg to about 44 mg/kg, about 28 mg/kg to about 42 mg/kg, about 28 mg/kg to about 40 mg/kg, about 28 mg/kg to about 38 mg/kg, about 28 mg/kg to about 36 mg/kg, about 28 mg/kg to about 34 mg/kg, about 28 mg/kg to about 32 mg/kg, about 28 mg/kg to about 30 mg/kg, about 30 mg/kg to about 50 mg/kg, about 30 mg/kg to about 48 mg/kg, about 30 mg/kg to about 46 mg/kg, about 30 mg/kg to about 44 mg/kg, about 30 mg/kg to about 42 mg/kg, about 30 mg/kg to about 40 mg/kg, about 30 mg/kg to about 38 mg/kg, about 30 mg/kg to about 36 mg/kg, about 30 mg/kg to about 34 mg/kg, about 30 mg/kg to about 32 mg/kg, about 32 mg/kg to about 50 mg/kg, about 32 mg/kg to about 48 mg/kg, about 32 mg/kg to about 46 mg/kg, about 32 mg/kg to about 44 mg/kg, about 32 mg/kg to about 42 mg/kg, about 32 mg/kg to about 40 mg/kg, about 32 mg/kg to about 38 mg/kg, about 32 mg/kg to about 36 mg/kg, about 32 mg/kg to about 34 mg/kg, about 34 mg/kg to about 50 mg/kg, about 34 mg/kg to about 48 mg/kg, about 34 mg/kg to about 46 mg/kg, about 34 mg/kg to about 44 mg/kg, about 34 mg/kg to about 42 mg/kg, about 34 mg/kg to about 40 mg/kg, about 34 mg/kg to about 38 mg/kg, about 34 mg/kg to about 36 mg/kg, about 36 mg/kg to about 50 mg/kg, about 36 mg/kg to about 48 mg/kg, about 36 mg/kg to about 46 mg/kg, about 36 mg/kg to about 44 mg/kg, about 36 mg/kg to about 42 mg/kg, about 36 mg/kg to about 40 mg/kg, about 36 mg/kg to about 38 mg/kg, about 38 mg/kg to about 50 mg/kg, about 38 mg/kg to about 48 mg/kg, about 38 mg/kg to about 46 mg/kg, about 38 mg/kg to about 44 mg/kg, about 38 mg/kg to about 42 mg/kg, about 38 mg/kg to about 40 mg/kg, about 40 mg/kg to about 50 mg/kg, about 40 mg/kg to about 48 mg/kg, about 40 mg/kg to about 46 mg/kg, about 40 mg/kg to about 44 mg/kg, about 40 mg/kg to about 42 mg/kg, about 42 mg/kg to about 50 mg/kg, about 42 mg/kg to about 48 mg/kg, about 42 mg/kg to about 46 mg/kg, about 42 mg/kg to about 44 mg/kg, about 44 mg/kg to about 50 mg/kg, about 44 mg/kg to about 48 mg/kg, about 44 mg/kg to about 46 mg/kg, about 46 mg/kg to about 50 mg/kg, about 46 mg/kg to about 48 mg/kg, or about 46 mg/kg to about 50 mg/kg)

In some embodiments, the methods described herein include administering to a subject about 10 mg/kg to about 400 mg/kg of body weight of sodium phenylbutyrate per day (e.g., about 10 mg/kg to about 380 mg/kg, about 10 mg/kg to about 360 mg/kg, about 10 mg/kg to about 340 mg/kg, about 10 mg/kg to about 320 mg/kg, about 10 mg/kg to about 300 mg/kg, about 10 mg/kg to about 280 mg/kg, about 10 mg/kg to about 260 mg/kg, about 10 mg/kg to about 240 mg/kg, about 10 mg/kg to about 220 mg/kg, about 10 mg/kg to about 200 mg/kg, about 10 mg/kg to about 180 mg/kg, about 10 mg/kg to about 160 mg/kg, about 10 mg/kg to about 140 mg/kg, about 10 mg/kg to about 120 mg/kg, about 10 mg/kg to about 100 mg/kg, about 10 mg/kg to about 80 mg/kg, about 10 mg/kg to about 60 mg/kg, about 10 mg/kg to about 40 mg/kg, about 10 mg/kg to about 20 mg/kg, about 20 mg/kg to about 400 mg/kg, about 20 mg/kg to about 380 mg/kg, about 20 mg/kg to about 360 mg/kg, about 20 mg/kg to about 340 mg/kg, about 20 mg/kg to about 320 mg/kg, about 20 mg/kg to about 300 mg/kg, about 20 mg/kg to about 280 mg/kg, about 20 mg/kg to about 260 mg/kg, about 20 mg/kg to about 240 mg/kg, about 20 mg/kg to about 220 mg/kg, about 20 mg/kg to about 200 mg/kg, about 20 mg/kg to about 180 mg/kg, about 20 mg/kg to about 160 mg/kg, about 20 mg/kg to about 140 mg/kg, about 20 mg/kg to about 120 mg/kg, about 20 mg/kg to about 100 mg/kg, about 20 mg/kg to about 80 mg/kg, about 20 mg/kg to about 60 mg/kg, about 20 mg/kg to about 40 mg/kg, about 40 mg/kg to about 400 mg/kg, about 40 mg/kg to about 380 mg/kg, about 40 mg/kg to about 360 mg/kg, about 40 mg/kg to about 340 mg/kg, about 40 mg/kg to about 320 mg/kg, about 40 mg/kg to about 300 mg/kg, about 40 mg/kg to about 280 mg/kg, about 40 mg/kg to about 260 mg/kg, about 40 mg/kg to about 240 mg/kg, about 40 mg/kg to about 220 mg/kg, about 40 mg/kg to about 200 mg/kg, about 40 mg/kg to about 180 mg/kg, about 40 mg/kg to about 160 mg/kg, about 40 mg/kg to about 140 mg/kg, about 40 mg/kg to about 120 mg/kg, about 40 mg/kg to about 100 mg/kg, about 40 mg/kg to about 80 mg/kg, about 40 mg/kg to about 60 mg/kg, about 60 mg/kg to about 400 mg/kg, about 60 mg/kg to about 380 mg/kg, about 60 mg/kg to about 360 mg/kg, about 60 mg/kg to about 340 mg/kg, about 60 mg/kg to about 320 mg/kg, about 60 mg/kg to about 300 mg/kg, about 60 mg/kg to about 280 mg/kg, about 60 mg/kg to about 260 mg/kg, about 60 mg/kg to about 240 mg/kg, about 60 mg/kg to about 220 mg/kg, about 60 mg/kg to about 200 mg/kg, about 60 mg/kg to about 180 mg/kg, about 60 mg/kg to about 160 mg/kg, about 60 mg/kg to about 140 mg/kg, about 60 mg/kg to about 120 mg/kg, about 60 mg/kg to about 100 mg/kg, about 60 mg/kg to about 80 mg/kg, about 80 mg/kg to about 400 mg/kg, about 80 mg/kg to about 380 mg/kg, about 80 mg/kg to about 360 mg/kg, about 80 mg/kg to about 340 mg/kg, about 80 mg/kg to about 320 mg/kg, about 80 mg/kg to about 300 mg/kg, about 80 mg/kg to about 280 mg/kg, about 80 mg/kg to about 260 mg/kg, about 80 mg/kg to about 240 mg/kg, about 80 mg/kg to about 220 mg/kg, about 80 mg/kg to about 200 mg/kg, about 80 mg/kg to about 180 mg/kg, about 80 mg/kg to about 160 mg/kg, about 80 mg/kg to about 140 mg/kg, about 80 mg/kg to about 120 mg/kg, about 80 mg/kg to about 100 mg/kg, about 100 mg/kg to about 400 mg/kg, about 100 mg/kg to about 380 mg/kg, about 100 mg/kg to about 360 mg/kg, about 100 mg/kg to about 340 mg/kg, about 100 mg/kg to about 320 mg/kg, about 100 mg/kg to about 300 mg/kg, about 100 mg/kg to about 280 mg/kg, about 100 mg/kg to about 260 mg/kg, about 100 mg/kg to about 240 mg/kg, about 100 mg/kg to about 220 mg/kg, about 100 mg/kg to about 200 mg/kg, about 100 mg/kg to about 180 mg/kg, about 100 mg/kg to about 160 mg/kg, about 100 mg/kg to about 140 mg/kg, about 100 mg/kg to about 120 mg/kg, about 120 mg/kg to about 400 mg/kg, about 120 mg/kg to about 380 mg/kg, about 120 mg/kg to about 360 mg/kg, about 120 mg/kg to about 340 mg/kg, about 120 mg/kg to about 320 mg/kg, about 120 mg/kg to about 300 mg/kg, about 120 mg/kg to about 280 mg/kg, about 120 mg/kg to about 260 mg/kg, about 120 mg/kg to about 240 mg/kg, about 120 mg/kg to about 220 mg/kg, about 120 mg/kg to about 200 mg/kg, about 120 mg/kg to about 180 mg/kg, about 120 mg/kg to about 160 mg/kg, about 120 mg/kg to about 140 mg/kg, about 140 mg/kg to about 400 mg/kg, about 140 mg/kg to about 380 mg/kg, about 140 mg/kg to about 360 mg/kg, about 140 mg/kg to about 340 mg/kg, about 140 mg/kg to about 320 mg/kg, about 140 mg/kg to about 300 mg/kg, about 140 mg/kg to about 280 mg/kg, about 140 mg/kg to about 260 mg/kg, about 140 mg/kg to about 240 mg/kg, about 140 mg/kg to about 220 mg/kg, about 140 mg/kg to about 200 mg/kg, about 140 mg/kg to about 180 mg/kg, about 140 mg/kg to about 160 mg/kg, about 160 mg/kg to about 400 mg/kg, about 160 mg/kg to about 380 mg/kg, about 160 mg/kg to about 360 mg/kg, about 160 mg/kg to about 340 mg/kg, about 160 mg/kg to about 320 mg/kg, about 160 mg/kg to about 300 mg/kg, about 160 mg/kg to about 280 mg/kg, about 160 mg/kg to about 260 mg/kg, about 160 mg/kg to about 240 mg/kg, about 160 mg/kg to about 220 mg/kg, about 160 mg/kg to about 200 mg/kg, about 160 mg/kg to about 180 mg/kg, about 180 mg/kg to about 400 mg/kg, about 180 mg/kg to about 380 mg/kg, about 180 mg/kg to about 360 mg/kg, about 180 mg/kg to about 340 mg/kg, about 180 mg/kg to about 320 mg/kg, about 180 mg/kg to about 300 mg/kg, about 180 mg/kg to about 280 mg/kg, about 180 mg/kg to about 260 mg/kg, about 180 mg/kg to about 240 mg/kg, about 180 mg/kg to about 220 mg/kg, about 180 mg/kg to about 200 mg/kg, about 200 mg/kg to about 400 mg/kg, about 200 mg/kg to about 380 mg/kg, about 200 mg/kg to about 360 mg/kg, about 200 mg/kg to about 340 mg/kg, about 200 mg/kg to about 320 mg/kg, about 200 mg/kg to about 300 mg/kg, about 200 mg/kg to about 280 mg/kg, about 200 mg/kg to about 260 mg/kg, about 200 mg/kg to about 240 mg/kg, about 200 mg/kg to about 220 mg/kg, about 220 mg/kg to about 400 mg/kg, about 220 mg/kg to about 380 mg/kg, about 220 mg/kg to about 360 mg/kg, about 220 mg/kg to about 340 mg/kg, about 220 mg/kg to about 320 mg/kg, about 220 mg/kg to about 300 mg/kg, about 220 mg/kg to about 280 mg/kg, about 220 mg/kg to about 260 mg/kg, about 220 mg/kg to about 240 mg/kg, about 240 mg/kg to about 400 mg/kg, about 240 mg/kg to about 380 mg/kg, about 240 mg/kg to about 360 mg/kg, about 240 mg/kg to about 340 mg/kg, about 240 mg/kg to about 320 mg/kg, about 240 mg/kg to about 300 mg/kg, about 240 mg/kg to about 280 mg/kg, about 240 mg/kg to about 260 mg/kg, about 260 mg/kg to about 400 mg/kg, about 260 mg/kg to about 380 mg/kg, about 260 mg/kg to about 360 mg/kg, about 260 mg/kg to about 340 mg/kg, about 260 mg/kg to about 320 mg/kg, about 260 mg/kg to about 300 mg/kg, about 260 mg/kg to about 280 mg/kg, about 280 mg/kg to about 400 mg/kg, about 280 mg/kg to about 380 mg/kg, about 280 mg/kg to about 360 mg/kg, about 280 mg/kg to about 340 mg/kg, about 280 mg/kg to about 320 mg/kg, about 280 mg/kg to about 300 mg/kg, about 300 mg/kg to about 400 mg/kg, about 300 mg/kg to about 380 mg/kg, about 300 mg/kg to about 360 mg/kg, about 300 mg/kg to about 340 mg/kg, about 300 mg/kg to about 320 mg/kg, about 320 mg/kg to about 400 mg/kg, about 320 mg/kg to about 380 mg/kg, about 320 mg/kg to about 360 mg/kg, about 320 mg/kg to about 340 mg/kg, about 340 mg/kg to about 400 mg/kg, about 340 mg/kg to about 380 mg/kg, about 340 mg/kg to about 360 mg/kg, about 360 mg/kg to about 400 mg/kg, about 360 mg/kg to about 380 mg/kg, or about 380 mg/kg to about 400 mg/kg)

In some embodiments, TURSO is administered in an amount of about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, or about 70 mg/kg of body weight per day. In some embodiments, sodium phenylbutyrate is administered in an amount of about 10 mg/kg, about 20 mg/kg, about 30 mg/kg, about 40 mg/kg, about 50 mg/kg, about 60 mg/kg, about 70 mg/kg, about 80 mg/kg, about 90 mg/kg, about 100 mg/kg, about 120 mg/kg, about 140 mg/kg, about 160 mg/kg, about 180 mg/kg, about 200 mg/kg, about 220 mg/kg, about 240 mg/kg, about 260 mg/kg, about 280 mg/kg, about 300 mg/kg, about 320 mg/kg, about 340 mg/kg, about 360 mg/kg, about 380 mg/kg, or about 400 mg/kg of body weight per day.

The methods described herein can be used for treating or ameliorating at least one symptom of ALS in a subject, slowing ALS disease progression, increasing survival time of a subject having one or more symptoms of ALS, preventing or reducing at least one adverse events (e.g., serious adverse events) associated with ALS or its treatment, and reducing the deterioration of, maintaining or improving muscle strength, respiratory muscle/pulmonary function and/or fine motor skill. The methods can also be used for prophylactically treating a subject at risk for developing ALS (e.g., a subject with a family history of ALS) or suspected to be developing ALS (e.g., a subject displaying at least one symptom of ALS, a symptom of upper motor neuron degeneration, and/or a symptom of lower motor neuron degeneration, but not enough symptoms at that time to support a full diagnosis of ALS). The methods are useful for ameliorating at least one symptom of lower motor neuron degeneration or upper motor neuron degeneration.

The methods disclosed herein are also useful for preventing or reducing constipation (e.g., constipation associated with ALS), and ameliorating at least one symptom of benign fasciculation syndrome or cramp fasciculation syndrome.

As disclosed herein, the methods can be used for treating a subject diagnosed with ALS, at risk for developing ALS, or suspected as having ALS. The subject may, for example, have been diagnosed with ALS for 24 months or less (e.g., any of the subranges within this range described herein). For example, the subject may have been diagnosed with ALS for 1 week or less, or on the same day that the presently disclosed treatments are administered. The subject may have shown one or more symptoms of ALS for 24 months or less (e.g., any of the subranges within this range described herein), have an ALS disease progression rate (ΔFS) of about 0.50 or greater (e.g., any of the subranges within this range described herein), have an ALSFRS-R score of 40 or less (e.g., any of the subranges within this range described herein), have lost on average about 0.8 to about 2 ALSFRS-R points per month (e.g. any of the subranges within this range described herein) over the previous 3-12 months, have a mutation in one or more genes selected from the group consisting of: SOD1, C9ORF72, ANG, TARDBP, VCP, VAPB, SQSTM1, DCTN1, FUS, UNC13A, ATXN2, HNRNPA1, CHCHD10, MOBP, C21ORF2, NEK1, TUBA4A, TBK1, MATR3, PFN1, UBQLN2, TAF15, OPTN, and TDP-43, and/or have a CSF or blood level of pNF-H of about 300 pg/mL or higher (e.g., about 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 3000, 3200, 3500, 3800, or 4000 pg/mL or higher). In some embodiments, the serum pNF-H level of subjects in the methods described herein can be about 70 to about 1200 pg/mL (e.g., about 70 to about 1000, about 70 to about 800, about 80 to about 600, or about 90 to about 400 pg/mL). In some embodiments, the CSF pNF-H levels of subjects in the methods described herein can be about 1000 to about 5000 pg/mL (e.g., about 1500 to about 4000, or about 2000 to about 3000 pg/mL). The subject may have a CSF or blood level of NfL of about 50 pg/mL or higher (e.g., about 60, 70, 80, 90, 100, 110, 120, 130, 140, 150, 160, 170, 180, 190, 200, 210, 220, 230, 240, or 250 pg/mL or higher). In some embodiments, the serum NfL level of subjects in the methods described herein can be about 50 to about 300 pg/mL (e.g., any of the subranges within this range described herein). In some embodiments, the CSF NfL level of subjects in the methods described herein can be about 2000 to about 40,000 pg/mL (e.g., any of the subranges within this range described herein).

Methods described in the present disclosure can include treatment of ALS per se, as well as treatment for one or more symptoms of ALS. “Treating” ALS does not require 100% abolition of the disease or disease symptoms in the subject. Any relief or reduction in the severity of symptoms or features of the disease is contemplated. “Treating” ALS also refers to a delay in onset of symptoms (e.g., in prophylaxis treatment) or delay in progression of symptoms or the loss of function associated with the disease. “Treating” ALS also refers to eliminating or reducing one or more side effects of a treatment (e.g. those caused by any of the therapeutic agents for treating ALS disclosed herein or known in the art). “Treating” ALS also refers to eliminating or reducing one or more direct or indirect effects of ALS disease progression, such as an increase in the number of falls, lacerations, or GI issues. The subject may not exhibit signs of ALS but may be at risk for ALS. For instance, the subject may carry mutations in genes associated with ALS, have family history of having ALS, or have elevated biomarker levels suggesting a risk of developing ALS. The subject may exhibit early signs of the disease or display symptoms of established or progressive disease. The disclosure contemplates any degree of delay in the onset of symptoms, alleviation of one or more symptoms of the disease, or delay in the progression of any one or more disease symptoms (e.g., any improvement as measured by ALSFRS-R, or maintenance of an ALSFRS-R rating (signaling delayed disease progression)). Any relief or reduction in the severity of symptoms or features of benign fasciculation syndrome and cramp-fasciculation syndrome are also contemplated herein.

The treatment provided in the present disclosure can be initiated at any stage during disease progression. For example, treatment can be initiated prior to onset (e.g., for subjects at risk for developing ALS), at symptom onset or immediately following detection of ALS symptoms, upon observation of any one or more symptoms (e.g., muscle weakness, muscle fasciculations, and/or muscle cramping) that would lead a skilled practitioner to suspect that the subject may be developing ALS. Treatment can also be initiated at later stages. For example, treatment may be initiated at progressive stages of the disease, e.g., when muscle weakness and atrophy spread to different parts of the body and the subject has increasing problems with moving. At or prior to treatment initiation, the subject may suffer from tight and stiff muscles (spasticity), from exaggerated reflexes (hyperreflexia), from muscle weakness and atrophy, from muscle cramps, and/or from fleeting twitches of muscles that can be seen under the skin (fasciculations), difficulty swallowing (dysphagia), speaking or forming words (dysarthria).

Treatment methods can include a single administration, multiple administrations, and repeating administration as required for the prophylaxis or treatment of ALS, or at least one symptom of ALS. The duration of prophylaxis treatment can be a single dosage or the treatment may continue (e.g., multiple dosages), e.g., for years or indefinitely for the lifespan of the subject. For example, a subject at risk for ALS may be treated with the methods provided herein for days, weeks, months, or even years so as to prevent the disease from occurring or fulminating. In some embodiments treatment methods can include assessing a level of disease in the subject prior to treatment, during treatment, and/or after treatment. The treatment provided herein can be administered one or more times daily, or it can be administered weekly or monthly. In some embodiments, treatment can continue until a decrease in the level of disease in the subject is detected. The methods provided herein may in some embodiments begin to show efficacy (e.g., alleviating one or more symptoms of ALS, improvement as measured by the ALSFRS-R, or maintenance of an ALSFRS-R rating) less than 60 days (e.g., less than 50, 45, 40, 35, 30, 25, 20, 15, or 10 days) after the initial administration, or after less than 60 administrations (e.g., less than 50, 45, 40, 35, 30, 25, 20, 15, or 10 administrations).

The terms “administer”, “administering”, or “administration” as used herein refers to administering drugs described herein to a subject using any art-known method, e.g., ingesting, injecting, implanting, absorbing, or inhaling, the drug, regardless of form. In some embodiments, one or more of the compounds disclosed herein can be administered to a subject by ingestion orally and/or topically (e.g., nasally). For example, the methods herein include administration of an effective amount of compound or compound composition to achieve the desired or stated effect. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician.

Following administration of the bile acid or a pharmaceutically acceptable salt thereof and the phenylbutyrate compound, the subject can be evaluated to detect, assess, or determine their level of ALS disease. In some embodiments, treatment can continue until a change (e.g., reduction) in the level of disease in the subject is detected.

Upon improvement of a patient's condition (e.g., a change (e.g., decrease) in the level of disease in the subject), a maintenance dose of a compound, composition or combination of this disclosure may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

### IV. Symptom and Outcome Measurements

This disclosure further provides methods of evaluating ALS symptoms, monitoring ALS progression and evaluating the subject's response to the treatment methods. Non-limiting examples include physical evaluation by a physician, weight, Electrocardiogram (ECG), ALS Functional Rating Scale (ALSFRS or ALSFRS-R) score, respiratory function, muscle strength, cognitive/behavioral function, quality of life, and speech analysis.

Respiratory function of the subject can be measured by e.g. vital capacity (including forced vital capacity and slow vital capacity), maximum mid-expiratory flow rate (MMERF), forced vital capacity (FVC), and forced expiratory volume in 1 second (FEV₁). Muscle strength can be evaluated by e.g. hand held dynamometry (HHD), hand grip strength dynamometry, manual muscle testing (MMT), electrical impedance myography (EIM), Maximum Voluntary Isometric Contraction Testing (MVICT), motor unit number estimation (MUNE), Accurate Test of Limb Isometric Strength (ATLIS), or a combination thereof. Cognitive/behavior function can be evaluated by e.g. the ALS Depression Inventory (ADI-12), the Beck Depression Inventory (BDI), and the Hospital Anxiety Depression Scale (HADS) questionnaires. Quality of life can be evaluated by e.g. the ALS Assessment Questionnaire (ALSAQ-40). The Akt level, Akt phosphorylation and/or pAktdAkt ratio can also be used to evaluate a subject's disease progression and response to treatment (See e.g., WO2012/160563).

The levels of biomarkers in the subject's CSF or blood samples are useful indicators of the subject's ALS progression and responsiveness to the methods of treatment provided herein. Biomarkers such as but not limited to, phosphorylated neurofilament heavy chain (pNF-H), neurofilament medium chain, neurofilament light chain (NFL), S100-β, cystatin C, chitotriosidase, CRP, TDP-43, uric acid, and certain micro RNAs, can be analyzed for this purpose. Urinalysis can also be used for assessing the subject's response to treatment. Levels of biomarkers such as but not limited to p75ECD and ketones in the urine sample can be analyzed. Levels of creatinine can be measured in the urine and blood samples. In some embodiments, the methods provided herein result in increased or decreased ketone levels in the subject's urine sample. Medical imaging, including but not limited to MRI and PET imaging of markers such as Translocator protein (TSPO), may also be utilized.

### Muscle Strength

The muscle strength of a subject can be evaluated using known methods in the art. Quantitative strength measures generally demonstrate a linear, predictable strength loss within an ALS patient. Tufts Quantitative Neuromuscular Examination (TQNE) can be used to provide quantitative measurements using a fixed strain gauge. TQNE measures isometric strength of 20 muscle groups and produces interval strength data in both strong and weak muscles (See e.g., Andres et al., Neurology 36:937-941, 1986). Hand-held dynamometry (HHD) tests isometric strength of specific muscles in the arms and legs and produces interval level data (See e.g., Shefne J M, Neurotherapeutics 14:154-160, 2017).

Accurate Test of Limb Isometric Strength (ATLIS) can be used to measure both strong and weak muscle groups using a fixed, wireless load cell (See e.g., Andres et al., Muscle Nerve 56(4):710-715, 2017). Force in twelve muscle groups are evaluated in an ATLIS testing, which reflect the subject's strength in the lower limbs, upper limbs, as well as the subject's grip strength. In some embodiments, ATLIS testing detects changes in muscle strength before any change in function is observed.

The methods provided herein may improve, maintain, or slow down the deterioration of a subject's muscle strength (e.g., lower limb strength, upper limb strength, or grip strength), as evaluated by any suitable methods described herein. The methods may result in improvement of the subject's upper limb strength more significantly than other muscle groups. For example, the effect on muscle strength can be reflected in one or more muscle groups selected from quadriceps, biceps, hamstrings, triceps, and anterior tibialis.

Muscle strength can be assessed by HHD, hand grip strength dynamometry, MMT, EIM, MVICT, MUNE, ATLIS, or a combination thereof, before, during and/or after the administration of a bile acid or a pharmaceutically acceptable salt thereof and a phenylbutyrate compound.

In some embodiments, the muscle strength is assessed by ATLIS. The total ATLIS score as well as the upper extremity and lower extremity ATLIS scores can be assessed. The methods of the present disclosure can result in a rate of decline in the total ATLIS score of a subject of about 3.50 PPN/month or less (e.g., about 3.45, 3.40, 3.35, 3.30, 3.25, 3.20, 3.15, 3.10, 3.05, 3.00 PPN/month or less). The methods of the present disclosure can also results in a reduction of the mean rate of decline in the total ATLIS score of a subject by at least about 0.2 PPN/month (e.g., at least about 0.25, 0.30, 0.35, 0.40, 0.45, or 0.50 PPN/month) as compared to a control subject not receiving the administration. The mean rate of decline in the upper extremity ATLIS score of a subject can be reduced by at least about 0.50 PPN/month (e.g., at least about 0.55, 0.60, 0.65, 0.70, 0.75, 0.80, 0.85, or 0.90 PPN/month) as compared to a control subject not receiving the administration described herein. The mean rate of decline in the lower extremity ATLIS score of a subject can be reduced by at least about 0.20 PPN/month (e.g., at least about 0.25, 0.30, 0.35, 0.40, 0.45, 0.50, 0.55, or 0.60 PPN/month) as compared to a control subject not receiving the administration described herein. In some embodiments, improvement or maintenance of the subject's muscle strength may begin to occur less than 60 days (e.g., less than 55, 50, 45, 40, 30, 25, or 20 days) following the initial administration. PPN represents the percentage of predicted normal strength based on age, sex weight and height.

### Pulmonary Function

ALS is a progressive neurodegenerative disease that ultimately leads to respiratory failure and death. Pulmonary function tests, such as vital capacity (VC), maximum mid-expiratory flow rate (MMERF), forced vital capacity (FVC), slow vital capacity (SVC), and forced expiratory volume in 1 second (FEV₁), can be used to monitor ALS progression and/or the subject's response to treatment. On average, the rate of respiratory function decline of an ALS patient measured by Vital Capacity (VC) can be about 2.24% of predicted (±6.9) per month. In some embodiments, measures from pulmonary function tests are associated with survival (See e.g., Moufavi et al. Iran J Neurol 13(3): 131-137, 2014). Additional measures, such as maximal inspiratory and expiratory pressures, arterial blood gas measurements, and overnight oximetry, may provide earlier evidence of dysfunction. Comparison of vital capacity in the upright and supine positions may also provide an earlier indication of weakening ventilatory muscle strength.

The methods provided herein may improve or maintain the subject's respiratory muscle and/or pulmonary function, or slow down the deterioration of the subject's respiratory muscle and/or pulmonary function. A subject's respiratory muscle and/or pulmonary function can be evaluated by any of the suitable methods described herein or otherwise known in the art. In some embodiments, the respiratory muscle function of a human subject is assessed based on the subject's SVC. In some embodiments of any of the methods of improving, maintaining, or slowing down the deterioration of respiratory muscle function in a human subject described herein, the treatment results in a mean rate of decline in the SVC of the subject of about 3.50 PPN/month or less (e.g., about 3.45, 3.40, 3.35, 3.30, 3.25, 3.20, 3.15, 3.10, 3.05, or 3.00 PPN/month or less). In some embodiments, the treatment reduces the mean rate of decline in the SVC of the subject by at least about 0.5 PPN/month (e.g., at least about 0.55, 0.60, 0.65, 0.70, 0.75, 0.80, 0.85, 0.90, 0.95, or 1.00 PPN/month) as compared to a control subject not receiving the treatment. In some embodiments, improvement or maintenance of the subject's pulmonary function may begin to occur less than 60 days (e.g., less than 55, 50, 45, 40, 30, 25, or 20 days) following the initial administration. In some embodiments, the subject's pulmonary function progresses less than expected after fewer than 60 days following the initial administration.

### Adverse Events

Subjects treated with any of the methods provided herein may present fewer adverse events (e.g., any of the adverse events disclosed herein), or present one or more of the adverse events to a lesser degree than control subjects not receiving the treatment. Exemplary adverse events include gastrointestinal related adverse events (e.g., abdominal pain, gastritis, nausea and vomiting, constipation, rectal bleeding, peptic ulcer disease, and pancreatitis); hematologic adverse events (e.g., aplastic anemia and ecchymosis); cardiovascular adverse events (e.g., arrhythmia and edema); renal adverse events (e.g., renal tubular acidosis); psychiatric adverse events (e.g., depression); skin adverse events (e.g., rash); and miscellaneous adverse events (e.g., syncope and weight gain). In some embodiments, the methods provided herein do not result in, or result in minimal symptoms of, constipation, neck pain, headache, falling, dry mouth, muscular weakness, falls, laceration, and Alanine Aminotransferase (ALT) increase. In some embodiments, the adverse events are serious adverse events, such as but not limited to respiratory adverse events, falls, or lacerations.

In some embodiments, administration of the combination of a bile acid and a phenylbutyrate compound can result in fewer adverse events (e.g., any of the adverse events disclosed herein), or less severe adverse events compared to administration of the bile acid or the phenylbutyrate compound alone.

The average survival time for an ALS patient may vary. The median survival time can be about 30 to about 32 months from symptom onset, or about 14 to about 20 months from diagnosis. The survival time of subjects with bulbar-onset ALS can be about 6 months to about 84 months from symptom onset, with a median of about 27 months. The methods provided herein may in some embodiments increase survival for a subject having ALS by at least one month (e.g., at least 2, 3, 4, 5, 6, 7, 8, 9, 10, 11, 12, 13, 14, 15, 16, 17, 18, 19, 20, 21, 22, 23, 24, 28, 32, 36, 40, 50, 60, 70, 80, or 90 months). Methods provided herein may in some embodiments delay the onset of ventilator-dependency or tracheostomy by at least one month (e.g., at least 2, 3, 4, 5, 6, 7, 8, 9, 10, 11, 12, 13, 14, 15, 16, 17, 18, 19, 20, 21, 22, 23, 24, 28, 32, 36, 40, 50, 60, 70, 80, or 90 months).

Methods provided herein may reduce disease progression rate wherein the average ALSFRS-R points lost per month by the subject is reduced by at least about 0.2 (e.g., at least about 0.25, 0.30, 0.35, 0.40, 0.45, 0.50, 0.55, 0.60, 0.65, 0.70, 0.75, 0.80, 0.85, 0.90, 0.95, 1.0, 1.05, 1.1, 1.15, 1.2, 1.25, 1.3, 1.35, 1.4, 1.45 or 1.5) as compared to a control subject not receiving the treatment. The methods provided herein may slow down the progression in one or more categories evaluated by the ALSFRS scale, including: speech, salivation, swallowing, handwriting, Cutting Food and Handling Utensils, Dressing and Hygiene, Turning in Bed and Adjusting Bed Clothes, Walking, Climbing Stairs, Dyspnea, Orthopnea, Respiratory Insufficiency. In some embodiments, the methods provided herein improve or slow down deterioration of a subject's fine motor function, as evaluated by one or more categories of the ALSFRS-R scale (e.g., handwriting, cutting food and handling utensils, or dressing and hygiene).

In some embodiments, the methods provided herein are more effective in treating subjects that are about 18 to about 50 years old (e.g., about 18 to about 45, about 18 to about 40, about 18 to about 35, about 18 to about 30, about 18 to about 25, or about 18 to about 22 years old), as compared to subjects 50 years or older (e.g., 55, 60, 65, 70, 75, or 80 years or older). In some embodiments, the methods provided herein are more effective in treating subjects who have been diagnosed with ALS and/or who showed ALS symptom onset less than about 24 months (e.g., less than about 22, 20, 18, 16, 14, 12, 10, 8, 6, 4, 2, or 1 month), as compared to subjects who has been diagnosed with ALS and/or who showed ALS symptom onset more than about 24 months (e.g., more than about 26, 28, 30, 32, 34, 36, 40, 45, 50, 55, or 60 months). In some embodiments, the methods provided herein are more effective in treating subjects who have been diagnosed with ALS and/or who showed ALS symptom onset more than about 24 months (e.g., more than about 26, 28, 30, 32, 34, 36, 40, 45, 50, 55, or 60 months), as compared to subjects who has been diagnosed with ALS and/or who showed ALS symptom less than about 24 months (e.g., less than about 22, 20, 18, 16, 14, 12, 10, 8, 6, 4, 2, or 1 month).

In some embodiments, responsiveness to the methods of treatment provided herein are gender-dependent. The methods provided herein can be more or less effective in treating female subjects as compared to male subjects. For instance, female subjects may show improvements (e.g., as measured by the ALSFRS-R or any other outcome measures described herein) earlier or later than male subjects when treated at similar stages of disease progression. Female subjects may in some embodiments show bigger or smaller improvements (e.g., as measured by the ALSFRS-R or any other outcome measures described herein) than male subjects when treated at similar stages of disease progression. The pharmacokinetics of the bile acid and the phenylbutyrate compound may be the same or different in female and male subjects.

### V. Additional Therapeutic Agents

The methods described herein can further include administering to the subject one or more additional therapeutic agents, e.g. in amounts effective for treating or achieving a modulation of at least one symptom of ALS. Any known ALS therapeutic agents known in the art can be used as an additional therapeutic agent. Exemplary therapeutic agents include riluzole (C₈H₅F₃N₂OS, e.g. sold under the trade names Rilutek® and Tiglutik®), edaravone (e.g. sold under the trade names Radicava® and Radicut®), dextromethorphan, anticholinergic medications, and psychiatric medications (e.g. antidepressants, antipsychotics, anxiolytics/hypnotics, mood stabilizers, and stimulants).

Neudexta® is a combination of dextromethorphan and quinidine, and can be used for the treatment of pseudobulbar affect (inappropriate laughing or crying). Anticholinergic medications and antidepressants can be used for treating excessive salivation. Examplary anticholinergic medications include glycopyrrolate, scopolamine, atropine (Atropen), belladonna alkaloids, benztropine mesylate (Cogentin), clidinium, cyclopentolate (Cyclogyl), darifenacin (Enablex), dicylomine, fesoterodine (Toviaz), flavoxate (Urispas), glycopyrrolate, homatropine hydrobromide, hyoscyamine (Levsinex), ipratropium (Atrovent), orphenadrine, oxybutynin (Ditropan XL), propantheline (Pro-banthine), scopolamine, methscopolamine, solifenacin (VESIcare), tiotropium (Spiriva), tolterodine (Detrol), trihexyphenidyl, trospium, and diphenhydramine (Benadryl). Exemplary antidepressants include selective serotonin inhibitors, serotonin-norepinephrine reuptake inhibitors, serotonin modulators and stimulators, serotonin antagonists and reuptake inhibitors, norepinephrine reuptake inhibitors, norepinephrine-dopamine reuptake inhibitors, tricyclic antidepressants, tetracyclic antidepressants, monoamine oxidase inhibitors, and NMDA receptor antagonists.

The additional therapeutic agent(s) can be administered for a period of time before administering the initial dose of a composition comprising a bile acid or a pharmaceutically acceptable salt thereof (e.g., TURSO) and a phenylbutyrate compound (e.g., sodium phenylbutyrate), and/or for a period of time after administering the final dose of the composition. In some embodiments, a subject in the methods described herein has been previously treated with one or more additional therapeutic agents (e.g., any of the additional therapeutic agents described herein, such as riluzole and edavarone). In some embodiments, the subject has been administered a stable dose of the therapeutic agent(s) (e.g., riluzole and/or edaravone) for at least 30 days (e.g., at least 40 days, 50 days, 60 days, 90 days, or 120 days) prior to administering the composition of the present disclosure. The absorption, metabolism, and/or excretion of the additional therapeutic agent(s) may be affected by the bile acid or a pharmaceutically acceptable salt thereof and/or the phenylbutyrate compound. For instance, co-administration of sodium phenylbutyrate with riluzole, or edavarone, may increase the subject's exposure to riluzole or edavarone. Co-administering riluzole with the bile acid or a pharmaceutically acceptable salt thereof and the phenylbutyrate compound can improve riluzole tolerance by the subject as compared to administering riluzole alone.

The combination of a bile acid or a pharmaceutically acceptable salt thereof, a phenylbutyrate compound, and one or more additional therapeutic agents can have a synergistic effect in treating ALS. Smaller doses of the additional therapeutic agents may be required to obtain the same pharmacological effect, when administered in combination with a bile acid or a pharmaceutically acceptable salt thereof, and a phenylbutyrate compound. In some embodiments, the amount of the additional therapeutic agent(s) administered in combination with a bile acid or a pharmaceutically acceptable salt thereof and a phenylbutyrate compound can be reduced by at least about 10% (e.g., at least about 15%, 20%, 25%, 30%, 35%, 40%, 45%, 50%, or 55%) compared to the dosage amount used when the additional therapeutic agent(s) is administered alone. Additionally or alternatively, the methods of the present disclosure can reduce the required frequency of administration of other therapeutic agents (e.g., other ALS therapeutic agents) to obtain the same pharmacological effect.

The bile acid or a pharmaceutically acceptable salt thereof and the phenylbutyrate compound can be administered shortly after a meal (e.g., within two hours of a meal) or under fasting conditions. The subject may have consumed food items (e.g., solid foods or liquid foods) less than 2 hours before administration of a bile acid or a pharmaceutically acceptable salt thereof and/or a phenylbutyrate compound; or will consume food items less than 2 hours after administration of one or both of the compounds. Food items may affect the rate and extent of absorption of the bile acid or a pharmaceutically acceptable salt thereof and/or the phenylbutyrate compound. For instance, food can change the bioavailability of the compounds by delaying gastric emptying, stimulating bile flow, changing gastrointestinal pH, increasing splanchnic blood flow, changing luminal metabolism of the substance, or physically or chemically interacting with a dosage form or the substance. The nutrient and caloric contents of the meal, the meal volume, and the meal temperature can cause physiological changes in the GI tract in a way that affects drug transit time, luminal dissolution, drug permeability, and systemic availability. In general, meals that are high in total calories and fat content are more likely to affect the GI physiology and thereby result in a larger effect on the bioavailability of a drug. The methods provided herein can further include administering to the subject a plurality of food items, for example, less than 2 hours (e.g., less than 1.5 hour, 1 hour, or 0.5 hour) before or after administering the bile acid or a pharmaceutically acceptable salt thereof, and/or the phenylbutyrate compound.

### EXAMPLES

Additional embodiments are disclosed in further detail in the following examples, which are provided by way of illustration and are not in any way intended to limit the scope of this disclosure or the claims.

### Example 1—Evaluation of the Potential for Induction of Cytochrome P450 Enzymes in Human Hepatocytes by Sodium Phenylbutyrate and Tauroursodeoxycholic Acid

The potential for cytotoxicity and induction of CYP mRNA (CYP1A2, CYP2B6 and CYP3A4) was evaluated in human hepatocytes by the combination of sodium phenylbutyrate (PB) and tauroursodeoxycholic acid (TUDCA).

### Cytotoxicity

The cytotoxicity of the combination of sodium phenylbutyrate (PB) and tauroursodeoxycholic acid (TUDCA) was evaluated using human hepatocytes. Plateable and inducible cryopreserved human hepatocytes were thawed and isolated in human hepatocyte thawing medium. The cells were suspended in human hepatocyte plating medium, counted (cell viability assessed by Trypan blue exclusion), seeded (Day 0) onto BioCoat™ collagen-coated 48-well plates (Corning Life Sciences, catalog #354505, Tewksbury, Mass.) at 0.75 million cells/mL (0.15 million cells/well in a 48-well plate), and incubated in a 95% air/5% CO2 incubator at 37° C. After attachment (4 hours), the medium was changed to fresh hepatocyte culture medium for overnight cell recovery. Hepatocytes were then treated with hepatocyte culture medium fortified with PB/TUDCA at five concentrations (PB/TUDCA, 14.8/1.62, 148/16.2, 826/108, 1480/1080, and 7400/1600 μM). A positive control (100 μM chlorpromazine) was treated in parallel. Vehicle controls were treated with hepatocyte culture medium containing the same content of solvent (1% MeOH for PB/TUDCA and 0.1% DMSO for chlorpromazine). The hepatocyte incubation was conducted in a 95% air/5% CO2 incubator at 37° C. for three days (72 hours) with daily replacement of the hepatocyte culture medium containing PB/TUDCA, positive control, or vehicles. The viability of cells was measured by analyzing the cellular conversion of tetrazolium compound [3-(4,5-dimethylthiazol-2-yl)-5-(3-arboxymethoxyphenyl)-2-(4-sulfophenyl)-2H tetrazolium, inner salt; MTS] into a formazan product by dehydrogenases, which are active only in viable cells. The absorbance of formazan, which is proportional to the number of viable cells, was measured spectrophotometrically using the CellTiter 96® AQueous One Solution Cell Proliferation Assay (MTS). Briefly, the wells were rinsed with DPBS, and then hepatocyte culture medium (200 μL) and the CellTiter 96® AQueous One Solution Cell Proliferation Assay reagent (40 μL) were added to each well, and the cells were incubated for 1 hour at 37° C. in a 95% air/5% CO2 incubator. The absorbance of formazan in each well was measured at 492 nm using a FLUOstar® OPTIMA Microplate Reader (BMG Lab Technologies, Durham, N.C., USA).

The cytotoxicity (expressed as cell viability, the percentage of MTS absorbance relative to the vehicle control) of PB/TUDCA and the positive control (100 μM chlorpromazine) in human hepatocytes is summarized in Table 1.

TABLE 1
Cell Viability after Treatment with PB/TUDCA or Positive Control

| | | Formazan | | |
|---|---|---|---|---|
| | | Absorbance | | % MTS Absorbance |
| Test | | (n = 3) | | Relative to |
| Article | Treatment | Mean | SD | Vehicle Control^{a} |
| Control | Vehicle | 0.659 | 0.00917 | 100 |
| | (0.1% DMSO) | | | |
| | Chlorpromazine | 0.160 | 0.000577 | 24.3 |
| PB/TUDCA | Vehicle | 0.646 | 0.0289 | 100 |
| | (1% MeOH) | | | |
| | PB/TUDCA | 0.241 | 0.0182 | 37.3 |
| | (7400/1600 μM) | | | |
| | PB/TUDCA | 0.349 | 0.0390 | 53.9 |
| | (1480/1080 μM) | | | |
| | PB/TUDCA | 0.458 | 0.0404 | 70.9 |
| | (826/108 μM) | | | |
| | PB/TUDCA | 0.622 | 0.0309 | 96.3 |
| | (148/16.2 μM) | | | |
| | PB/TUDCA | 0.690 | 0.0217 | 107 |
| | (14.8/1.62 μM) | | | |

^{a}MTS absorbance <75% of the vehicle control is considered a positive cytotoxic result. QC acceptance criterion: % MTS absorbance relative to the vehicle control ≤50 by positive control.

The cytotoxicity of the combination of PB and TUDCA at five concentrations (PB/TUDCA, 14.8/1.62, 148/16.2, 826/108, 1480/1080, and 7400/1600 μM) was evaluated using one human hepatocyte donor. The results showed that PB/TUDCA had no cytotoxicity at 14.8/1.62 μM and 148/16.2 μM, but showed cytotoxicity at 826/108 μM, 1480/1080 μM, and 7400/1600 μM for the donor. Because the cytotoxicity results were marginal at 826/108 μM PB/TUDCA, this combination of test concentrations was included in the subsequent CYP induction test (using a different donor).

### CYP Induction

Plateable and inducible cryopreserved human hepatocytes were thawed and isolated in human hepatocyte thawing medium. The cells were suspended in human hepatocyte plating medium, counted (cell viability assessed by Trypan blue exclusion), seeded (Day 0) onto BioCoat™ collagen-coated 48-well plates (Corning Life Sciences, catalog #354505) at 0.75 million cells/mL (0.15 million cells/well in a 48-well plate), and incubated in a 95% air/5% CO2 incubator at 37° C. After attachment (4 hours), the medium was changed to fresh hepatocyte culture medium for overnight cell recovery. Hepatocytes were then treated with hepatocyte culture medium fortified with PB/TUDCA at three concentrations (14.8/1.62, 148/16.2, and 826/108 μM, based on the cytotoxicity test results).

Positive controls were treated in parallel with hepatocyte culture medium fortified with a known inducer of each CYP of interest: 50 μM omeprazole (OME) for CYP1A2, 1,000 μM phenobarbital for CYP2B6, or 50 μM rifampicin (RIF) for CYP3A4. Negative controls were treated with 10 μM flumazenil, and vehicle controls were treated with hepatocyte culture medium. All experiments were performed in triplicate. The hepatocyte incubation was conducted in a 95% air/5% CO2 incubator at 37° C. for three days (72 hours) with daily replacement of the hepatocyte culture medium containing TA, positive or negative inducer, or vehicle. The experimental conditions for CYP induction and sample treatment are summarized in Table 2.

TABLE 2
CYP Induction Conditions

| | |
|---|---|
| TA Concentrations | PB/TUDCA, 14.8/1.62, 148/16.2, and 826/108 μM |
| Positive Controls | 50 μM OME for CYP1A2; 1,000 μM phenobarbital |
| | for CYP2B6; 50 μM RIF for CYP3A4, CYP2C8, |
| | CYP2C9, and CYP2C19 |
| Negative Control | 10 μM flumazenil for all CYPs |
| Vehicle Control | Hepatocyte culture medium |
| Incubation | 37° C. |
| Temperature | |
| Incubator | 95% air/5% CO₂ |
| Conditions | |
| Induction Period | 72 hours with daily replacement of hepatocyte |
| | culture medium containing TA, positive or negative |
| | inducer, or vehicle |

### Cell Viability Assay

After the CYP induction treatment, the cells were used for cell viability assay. The viability of cells (expressed as the percentage of MTS absorbance relative to vehicle control) was measured as described herein.

qPCR-mRNA Assay

After induction treatment, CYP mRNA expression was measured by qPCR. Total RNA was isolated from the treated cells using the RNeasy® mini kit (Qiagen, Valencia, Calif., USA) and treated with RNase-free DNase (Qiagen) following the manufacturer's protocols. The concentration of RNA was determined using a Qubit® Fluorometer with a Qubit RNA HS assay kit (Invitrogen). cDNA was synthesized from up to 1 μg of the total RNA harvested from the cells using a QuantiTect® RT kit (Qiagen). Analysis of CYP gene expression by qPCR (Table 3) was performed using the LightCycler® 480 II System (Roche Diagnostics Corporation, Indianapolis, Ind., USA).

TABLE 3

| Gene | Forward Primer | Reverse Primer | Probe |
|---|---|---|---|
| CYP1A2 | ccagctgccctacttgga | gtgtcccttgttgtgctgtg | cctggaga (SEQ ID NO: 3) |
| | (SEQ ID NO: 1) | (SEQ ID NO: 2) | |
| CYP2B6 | acttcgggatgggaaagc | gaggaaggtggggtccat | tggaggag (SEQ ID NO: 6) |
| | (SEQ ID NO: 4) | (SEQ ID NO: 5) | |
| CYP3A4 | gatggetctcatcccagactt | agtccatgtgaatgggttcc | ttctcctg (SEQ ID NO: 9) |
| | (SEQ ID NO: 7) | (SEQ ID NO: 8) | |

Relative mRNA was expressed as the fold-increase calculated from the normalized mRNA level (2^{-ΔΔCt}) relative to vehicle control. The percentage of mRNA fold-increase relative to positive control was calculated using the following equation:

% Induction relative to positive control=100×(mRNATA−mRNAvehicle)/(mRNApositive control−mRNAvehicle)

The induction of CYP1A2, CYP2B6, and CYP3A4 mRNA by PB and TUDCA at three concentrations (PB/TUDCA at 14.8/1.62, 148/16.2, and 826/108 μM) was evaluated using three human hepatocyte donors. In donor 1, PB and TUDCA did not increase the mRNA of CYP1A2 and CYP3A4 (<2-fold vs. vehicle control and <20% of the positive control) at any of the three tested concentrations. PB and TUDCA did not increase the mRNA of CYP2B6 at 14.8/1.62 and 148/16.2 μM, but 5.56-fold induction was observed at 826/108 μM.

In donor 2, PB and TUDCA did not increase the mRNA of CYP1A2 and CYP3A4 at any of the three tested concentrations. PB and TUDCA did not increase the mRNA of CYP2B6 at 14.8/1.62 μM, but 3.18- and 6.82-fold induction was observed at 148/16.2 μM and 826/108 μM, respectively.

In donor 3, PB and TUDCA did not increase the mRNA of CYP1A2 and CYP3A4 at any of the three tested concentrations. PB and TUDCA did not increase the mRNA of CYP2B6 at 14.8/1.62 and 148/16.2 μM, but 6.66-fold induction was observed at 826/108 μM.

TABLE 4
Induction of CYP1A2 mRNA

| | | | | | % Induction |
|---|---|---|---|---|---|
| | | | Fold-increase^{a} | | Relative to |
| | Test | | (n = 3) | | Positive |
| Donor | Article | Treatment | Mean | SD | Control^{b} |
| 1 | Control | Vehicle | 1.00 | 0.115 | 0 |
| | | OME | 78.7 | 3.02 | 100 |
| | | Flumazenil | 0.424 | 0.239 | 0 |
| | PB/TUDCA | PB/TUDCA | 0.489 | 0.230 | 0 |
| | | (826/108 μM) | | | |
| | | PB/TUDCA | 0.238 | 0.0307 | 0 |
| | | (148/16.2 μM) | | | |
| | | PB/TUDCA | 0.257 | 0.0780 | 0 |
| | | (14.8/1.62 μM) | | | |
| 2 | Control | Vehicle | 1.03 | 0.300 | 0 |
| | | OME | 20.8 | 6.34 | 100 |
| | | Flumazenil | 1.20 | 0.288 | 0.899 |
| | PB/TUDCA | PB/TUDCA | 1.32 | 0.426 | 1.47 |
| | | (826/108 μM) | | | |
| | | PB/TUDCA | 0.584 | 0.0283 | 0 |
| | | (148/16.2 μM) | | | |
| | | PB/TUDCA | 0.569 | 0.188 | 0 |
| | | (14.8/1.62 μM) | | | |
| 3 | Control | Vehicle | 1.03 | 0.277 | 0 |
| | | OME | 17.5 | 0.745 | 100 |
| | | Flumazenil | 1.05 | 0.0193 | 0.117 |
| | PB/TUDCA | PB/TUDCA | 1.28 | 0.173 | 1.54 |
| | | (826/108 μM) | | | |
| | | PB/TUDCA | 0.612 | 0.0814 | 0 |
| | | (148/16.2 μM) | | | |
| | | PB/TUDCA | 0.560 | 0.211 | 0 |
| | | (14.8/1.62 μM) | | | |

^{a}Fold-increase was calculated from the normalized mRNA level (2^{-ΔΔCt}) of TA-, positive inducer-, or negative inducer-treated cells relative to that of vehicle-treated cells.
^{b}Percentage of mRNA fold-increase relative to positive inducer-treated cells. Negative values are treated as zero.

TABLE 5
Induction of CYP2B6 mRNA

| | | | | | % Induction |
|---|---|---|---|---|---|
| | | | Fold-increase^{a} | | Relative to |
| | Test | | (n = 3) | | Positive |
| Donor | Article | Treatment | Mean | SD | Control^{b} |
| 1 | Control | Vehicle | 1.01 | 0.186 | 0 |
| | | Phenobarbital | 21.8 | 0.777 | 100 |
| | | Flumazenil | 0.866 | 0.203 | 0 |
| | PB/TUDCA | PB/TUDCA | 5.56 | 1.03 | 21.8 |
| | | (826/108 μM) | | | |
| | | PB/TUDCA | 1.22 | 0.175 | 1.00 |
| | | (148/16.2 μM) | | | |
| | | PB/TUDCA | 0.718 | 0.109 | 0 |
| | | (14.8/1.62 μM) | | | |
| 2 | Control | Vehicle | 1.00 | 0.0629 | 0 |
| | | Phenobarbital | 39.6 | 11.1 | 100 |
| | | Flumazenil | 1.23 | 0.321 | 0.594 |
| | PB/TUDCA | PB/TUDCA | 6.82 | 0.816 | 15.1 |
| | | (826/108 μM) | | | |
| | | PB/TUDCA | 3.18 | 0.502 | 5.65 |
| | | (148/16.2 μM) | | | |
| | | PB/TUDCA | 0.943 | 0.0620 | 0 |
| | | (14.8/1.62 μM) | | | |
| 3 | Control | Vehicle | 1.06 | 0.455 | 0 |
| | | Phenobarbital | 7.36 | 0.599 | 100 |
| | | Flumazenil | 0.915 | 0.0507 | 0 |
| | PB/TUDCA | PB/TUDCA | 6.66 | 0.959 | 88.9 |
| | | (826/108 μM) | | | |
| | | PB/TUDCA | 1.63 | 0.0623 | 9.04 |
| | | (148/16.2 μM) | | | |
| | | PB/TUDCA | 0.902 | 0.397 | 0 |
| | | (14.8/1.62 μM) | | | |

TABLE 6
Induction of CYP3A4 mRNA

| | | | | | % Induction |
|---|---|---|---|---|---|
| | | | Fold-increase^{a} | | Relative to |
| | Test | | (n = 3) | | Positive |
| Donor | Article | Treatment | Mean | SD | Control^{b} |
| 1 | Control | Vehicle | 1.00 | 0.115 | 0 |
| | | RIF | 19.7 | 1.71 | 100 |
| | | Flumazenil | 0.903 | 0.0734 | 0 |
| | PB/TUDCA | PB/TUDCA | 0.991 | 0.0137 | 0 |
| | | (826/108 μM) | | | |
| | | PB/TUDCA | 1.04 | 0.0183 | 0.217 |
| | | (148/16.2 μM) | | | |
| | | PB/TUDCA | 0.724 | 0.0220 | 0 |
| | | (14.8/1.62 μM) | | | |
| 2 | Control | Vehicle | 1.00 | 0.0778 | 0 |
| | | RIF | 43.2 | 5.10 | 100 |
| | | Flumazenil | 1.30 | 0.179 | 0.701 |
| | PB/TUDCA | PB/TUDCA | 1.06 | 0.195 | 0.131 |
| | | (826/108 μM) | | | |
| | | PB/TUDCA | 1.33 | 0.151 | 0.780 |
| | | (148/16.2 μM) | | | |
| | | PB/TUDCA | 1.18 | 0.228 | 0.417 |
| | | (14.8/1.62 μM) | | | |
| 3 | Control | Vehicle | 1.01 | 0.138 | 0 |
| | | RIF | 7.70 | 1.27 | 100 |
| | | Flumazenil | 0.788 | 0.166 | 0 |
| | PB/TUDCA | PB/TUDCA | 1.23 | 0.0406 | 3.36 |
| | | (826/108 μM) | | | |
| | | PB/TUDCA | 0.752 | 0.0748 | 0 |
| | | (148/16.2 μM) | | | |
| | | PB/TUDCA | 0.687 | 0.0946 | 0 |
| | | (14.8/1.62 μM) | | | |

TABLE 7
Cell Viability after CYP Induction Treatment

| | | | Formazan | | % MTS Absorbance |
|---|---|---|---|---|---|
| | | | Absorbance (n = 6) | | Relative to Vehicle |
| Donor | Test Article | Treatment | Mean | SD | Control |
| 1 | Control | Vehicle | 0.426 | 0.0225 | 100 |
| | | OME | 0.488 | 0.0144 | 114 |
| | | Phenobarbital | 0.417 | 0.0304 | 97.7 |
| | | RIF | 0.544 | 0.0153 | 127 |
| | | Flumazenil | 0.432 | 0.0158 | 101 |
| | PB/TUDCA | PB/TUDCA | 0.334 | 0.0205 | 78.2 |
| | | (826/108 μM) | | | |
| | | PB/TUDCA | 0.407 | 0.0184 | 95.5 |
| | | (148/16.2 μM) | | | |
| | | PB/TUDCA | 0.413 | 0.0269 | 96.8 |
| | | (14.8/1.62 μM) | | | |
| 2 | Control | Vehicle | 0.510 | 0.0377 | 100 |
| | | OME | 0.586 | 0.0268 | 115 |
| | | Phenobarbital | 0.504 | 0.0531 | 98.8 |
| | | RIF | 0.640 | 0.0310 | 126 |
| | | Flumazenil | 0.527 | 0.0293 | 103 |
| | PB/TUDCA | PB/TUDCA | 0.336 | 0.0118 | 55.9 |
| | | (826/108 μM) | | | |
| | | PB/TUDCA | 0.474 | 0.0570 | 93.1 |
| | | (148/16.2 μM) | | | |
| | | PB/TUDCA | 0.480 | 0.0337 | 94.2 |
| | | (14.8/1.62 μM) | | | |
| 3 | Control | Vehicle | 0.296 | 0.0528 | 100 |
| | | OME | 0.341 | 0.0473 | 115 |
| | | Phenobarbital | 0.320 | 0.0550 | 108 |
| | | RIF | 0.378 | 0.0416 | 128 |
| | | Flumazenil | 0.337 | 0.0359 | 114 |
| | PB/TUDCA | PB/TUDCA | 0.276 | 0.0400 | 93.2 |
| | | (826/108 μM) | | | |
| | | PB/TUDCA | 0.299 | 0.0654 | 101 |
| | | (148/16.2 μM) | | | |
| | | PB/TUDCA | 0.307 | 0.0670 | 104 |
| | | (14.8/1.62 (μM) | | | |

In conclusion, PB and TUDCA are unlikely inducers of CYP1A2 or CYP3A4 in the concentration range from 14.8/1.62 μM to 826/108 μM. Induction of CYP2B6 by PB and TUDCA were observed, mainly at 826/108 μM.

### Example 2—Inhibition of Cytochrome P450 Enzymes in Human Liver Microsomes by Sodium Phenylbutyrate and Tauroursodeoxycholic Acid

The inhibition of cytochrome P450 (CYP) enzymes (CYP1A2, CYP2B36, CYP2C8, CYP2C9, CYP2C19, CYP2D6, and CYP3A) in human liver microsomes (HLM) by sodium phenylbutyrate (PB) and tauroursodeoxycholic acid (TUDCA) was evaluated. PB and TUDCA at eight concentrations were co-incubated with pooled HLM (0.25 mg protein/mL) in phosphate buffer (100 mM, pH 7.4) containing MgCl2 (5 mM), CYP-specific probe substrate (at approximately the Kₘ), and NADPH (1 mM). After a period of incubation, the reaction was terminated by the addition of ice-cold protein precipitation solvent (acetonitrile containing an internal standard) and the samples were centrifuged. CYP enzyme activity was determined by measuring the formation of each CYP probe metabolite by LC-MS/MS.

PB (0-7400 μM, 50×C_{max,u}=7400 μM, 0.1×I_{gut}=6444 μM) and TUDCA (0-1600 μM, 50×C_{max,u}=81 μM, 0.1×I_{gut}=1600 μM) at eight concentrations per test article were incubated (co-incubation of PB and TUDCA) with pooled HLM (0.25 mg protein/mL) in phosphate buffer (100 mM, pH 7.4) containing MgCl2 (5 mM), NADPH (1 mM), and an individual CYP probe substrate. The total organic solvent content in the final incubation was less than 1% (DMSO ≤0.1%, other organic solvent ≤1%). The reaction mixture without NADPH was equilibrated in a shaking water bath at 37° C. for 5 minutes. The reaction was initiated by the addition of NADPH, followed by incubation at 37° C. for 10-30 minutes depending on the individual CYP isoform. The reaction was terminated by the addition of two volumes of ice-cold acetonitrile (ACN) containing an internal standard (IS, stable isotope-labeled CYP probe metabolite). Negative (vehicle) controls were conducted without TA. Positive controls were performed in parallel using known CYP inhibitors. After the removal of protein by centrifugation at 1,640 g for 10 minutes at 4° C., the supernatants were transferred to an HPLC autosampler plate. The formation of individual CYP probe metabolites was determined by LC-MS/MS. The experimental conditions for CYP reaction and sample analysis are summarized in Table 8.

The percent of control enzyme activity was calculated using the following equation:

% of control enzyme activity=100×(enzyme activity in the presence of TA/enzyme activity in the absence of TA)

TABLE 8
CYP Reaction and Sample Analysis

| | |
|---|---|
| Incubation | PB: 0-7400 μM, TUDCA: 0-1600 μM; HLM: 0.25 mg |
| Composition | protein/mL; Phosphate buffer: 100 mM, pH 7.4; |
| | MgCl₂: 5 mM; Probe substrate: ~Kₘ |
| Equilibration | 37° C. for 5 min |
| NADPH | 1 mM |
| Incubation | 37° C. for 10 min (CYP3A), 30 min (CYP2C19), or 20 |
| Time | min (all other CYPs) |
| Reaction | ACN containing IS (1:2, v/v) |
| Termination | |
| Sample | Centrifugation at 1,640 g for 10 min at 4° C. |
| Treatment | |
| Sample | LC-MS/MS |
| Analysis | |

The enzyme activity was expressed as the peak area ratio of probe metabolite to IS, measured by LC-MS/MS. The IC50 value was estimated by fitting the experimental data (percent enzyme activity of control vs. log [inhibitor concentration] to a sigmoidal model, followed by non-linear regression analysis using GraphPad Prism (Version 5.0 or higher, GraphPad Software, San Diego, Calif., USA).

The inhibition of CYP activities in HLM by PB and TUDCA is summarized in Table 9. The inhibition of CYP activities by positive inhibitors is summarized in Table 10.

TABLE 9
Inhibition of CYP Activities in HLM by PB and TUDCA

| | % of Control Enzyme Activity (n = 3)^{a} | | | | | | | | |
|---|---|---|---|---|---|---|---|---|---|
| | PB (μM) | 0 | 10.2 | 30.5 | 91.4 | 274 | 822 | 2467 | 7400 |
| CYP | TUDCA (μM) | 0 | 2.19 | 6.58 | 19.8 | 59.3 | 178 | 533 | 1600 |
| CYP1A2 | Average | 100 | 97.0 | 97.1 | 101 | 99.9 | 93.3 | 82.2 | 6.44 |
| | SD | 1.74 | 0.808 | 3.32 | 3.16 | 5.43 | 2.58 | 5.34 | 4.20 |
| CYP2B6 | Average | 100 | 101 | 96.1 | 94.3 | 81.1 | 62.7 | 26.4 | 3.39 |
| | SD | 3.26 | 4.64 | 2.81 | 3.26 | 2.63 | 1.03 | 1.45 | 1.06 |
| CYP2C8 | Average | 100 | 95.9 | 97.2 | 95.2 | 93.5 | 88.0 | 74.0 | 30.1 |
| | SD | 1.93 | 3.31 | 3.45 | 3.83 | 1.60 | 1.38 | 4.96 | 1.87 |
| CYP2C9 | Average | 100 | 101 | 103 | 99.7 | 88.9 | 69.3 | 50.1 | 32.0 |
| | SD | 3.05 | 0.605 | 6.40 | 4.02 | 4.29 | 3.74 | 0.398 | 1.75 |
| CYP2C19 | Average | 100 | 101 | 98.3 | 93.1 | 90.2 | 84.7 | 75.8 | 46.1 |
| | SD | 4.18 | 7.56 | 0.681 | 2.59 | 9.50 | 3.14 | 6.36 | 2.12 |
| CYP2D6 | Average | 100 | 102 | 100 | 101 | 96.1 | 98.5 | 92.2 | 49.6 |
| | SD | 2.18 | 3.96 | 3.06 | 4.51 | 1.34 | 3.38 | 4.41 | 2.76 |
| CYP3A | Average | 100 | 99.1 | 100 | 97.9 | 95.3 | 83.0 | 62.9 | 14.6 |
| (Midazolam) | SD | 1.26 | 0.529 | 1.74 | 1.74 | 0.378 | 1.32 | 0.471 | 1.51 |
| CYP3A | Average | 100 | 99.7 | 102 | 97.5 | 98.0 | 93.1 | 73.0 | 23.0 |
| (Testosterone) | SD | 3.08 | 2.53 | 5.32 | 3.61 | 4.02 | 5.68 | 5.62 | 1.13 |

^{a}Percent of control enzyme activity = 100 × (Enzyme activity in the presence of TA/Enzyme activity in the absence of TA). Enzyme activity was calculated from the peak area ratio of CYP probe metabolite to IS by LC-MS/MS.
SD: standard deviation.

TABLE 10
Inhibition of CYP Activities in HLM by Positive Inhibitors

| | | | | | | | | | | IC₅₀ |
|---|---|---|---|---|---|---|---|---|---|---|
| CYP | % of Control Enzyme Activity (n = 3)ª | | | | | | | | | (μM) |
| CYP1A2 | α-Naphthoflavone (μM) | 0 | 0.00137 | 0.00412 | 0.0123 | 0.0370 | 0.111 | 0.333 | 1.00 | 0.0242 |
| | Average | 100 | 102 | 90.5 | 73.0 | 36.7 | 12.8 | 7.15 | 5.62 | |
| | SD | 3.96 | 5.44 | 7.36 | 6.11 | 1.05 | 0.562 | 0.99 | 0.361 | |
| CYP2B6 | Thio-TEPA (μM) | 0 | 0.0412 | 0.123 | 0.370 | 1.11 | 3.33 | 10.0 | 30.0 | 7.30 |
| | Average | 100 | 95.0 | 86.1 | 89.3 | 76.1 | 66.9 | 44.1 | 26.7 | |
| | SD | 9.61 | 7.16 | 4.50 | 3.30 | 1.45 | 4.40 | 1.23 | 0.158 | |
| CYP2CS | Montelukast (μM) | 0 | 0.0137 | 0.0412 | 0.123 | 0.370 | 1.11 | 3.33 | 10.0 | 0.517 |
| | Average | 100 | 94.2 | 89.9 | 78.9 | 58.7 | 31.2 | 14.6 | 5.31 | |
| | SD | 6.86 | 1.15 | 5.51 | 1.79 | 2.82 | 1.00 | 0.934 | 0.334 | |
| CYP2C9 | Sulfaphenazole (μM) | 0 | 0.0137 | 0.0412 | 0.123 | 0.370 | 1.11 | 3.33 | 10.0 | 0.687 |
| | Average | 100 | 95.5 | 90.4 | 82.1 | 63.6 | 39.3 | 23.8 | 14.9 | |
| | SD | 2.74 | 1.46 | 0.256 | 1.35 | 2.48 | 0.284 | 1.25 | 0.292 | |
| CYP2C19 | (+)-N-3- | 0 | 0.0412 | 0.123 | 0.370 | 1.11 | 3.33 | 10.0 | 30.0 | 0.293 |
| | benzylnirvanol (μM) | | | | | | | | | |
| | Average | 100 | 83.5 | 68.8 | 44.1 | 23.1 | 11.5 | 6.17 | 3.18 | |
| | SD | 6.99 | 4.97 | 6.76 | 4.58 | 2.22 | 3.09 | 1.10 | 0.617 | |
| CYP2D6 | Quinidine (μM) | 0 | 0.0137 | 0.0412 | 0.123 | 0.370 | 1.11 | 3.33 | 10.0 | 0.0632 |
| | Average | 100 | 79.4 | 57.6 | 35.8 | 21.6 | 11.5 | 7.90 | 5.61 | |
| | SD | 7.03 | 3.61 | 0.261 | 0.300 | 0.594 | 0.0876 | 0.444 | 0.210 | |
| CYP3A | Ketoconazole (μM) | 0 | 0.00412 | 0.0123 | 0.0370 | 0.111 | 0.333 | 1.00 | 3.00 | 0.0243 |
| (Midazolam) | Average | 100 | 89.8 | 69.1 | 37.4 | 19.0 | 8.64 | 3.74 | 1.51 | |
| | SD | 2.97 | 2.90 | 2.52 | 1.05 | 1.81 | 0.679 | 0.416 | 0.108 | |
| CYP3A | Ketoconazole (μM) | 0 | 0.00412 | 0.0123 | 0.0370 | 0.111 | 0.333 | 1.00 | 3.00 | 0.0130 |
| (Testosterone) | Average | 100 | 74.8 | 53.1 | 24.7 | 11.0 | 4.97 | 2.72 | 2.22 | |
| | SD | 6.74 | 5.44 | 2.72 | 0.762 | 1.42 | 0.487 | 0.617 | 0.307 | |

The combination of PB (up to 274 μM) and TUDCA (up to 59.3 μM) showed little or no inhibition (<20%) of any of the seven major CYPs tested. The combination of PB (at 822 μM) and TUDCA (at 178 μM) showed 7% inhibition of CYP1A2, 12% inhibition of CYP2C8, 15% inhibition of CYP2C19, 1.5% inhibition of CYP2D6, 7-17% inhibition of CYP3A, 37% inhibition of CYP2B6, and 31% inhibition of CYP2C9. The combination of PB (at 2467 μM) and TUDCA (at 533 μM) showed 18% inhibition of CYP1A2, 8% inhibition of CYP2D6, 74% inhibition of CYP2B6, 26% inhibition of CYP2C8, 50% inhibition of CYP2C9, 24% inhibition of CYP2C19, and 27-37% inhibition of CYP3A. The combination of PB (at 7400 μM) and TUDCA (at 1600 μM) (the highest tested concentrations in this study) showed >50% inhibition of all of the seven major CYPs tested.

### Example 3—Cytochrome P450 Reaction Phenotyping of Sodium Phenylbutyrate and Tauroursodeoxycholic Acid Using Human Liver Microsomes and Chemical Inhibitors

The objective of this study was to perform cytochrome P450 (CYP) reaction phenotyping of sodium phenylbutyrate (PB) and tauroursodeoxycholic acid (TUDCA) using human liver microsomes (HLM) and chemical inhibitors.

CYP reaction phenotyping of PB and TUDCA was evaluated using pooled human liver microsomes (HLM, 0.5 mg protein/mL) and CYP-selective inhibitors. The amounts of PB and TUDCA remaining after a period of incubation (0, 5, 10, 20, 30, and 60 minutes) were measured by LC-MS/MS.

After 60 minutes of incubation with HLM, the percent of PB and TUDCA remaining was 95% or greater, in the absence and presence of NADPH, with or without 15 minutes of pre-incubation. These results suggest that neither PB nor TUDCA is metabolized by the major CYPs in HLM.

Methods

PB and TUDCA, at one concentration each (5 μM in the final incubation), were co-incubated with HLM (0.5 mg protein/mL) in phosphate buffer (100 mM, pH 7.4) containing MgCl2 (5 mM) and NADPH (1 mM), in the absence and presence of an individual CYP-selective inhibitor (Table 11). For reversible inhibition, the reaction mixture, without NADPH, was equilibrated in a shaking water bath at 37° C. for 5 minutes, and the reaction was initiated by the addition of NADPH (except the control without NADPH), followed by incubation at 37° C. For irreversible inhibition, the inhibitors were pre-incubated with HLM in the presence of NADPH at 37° C. for 15 minutes and the reaction was initiated by the addition of the TA. Aliquots of the incubated solutions were sampled at 0, 5, 10, 20, 30, and 60 minutes. The reaction was terminated by the addition of ice-cold acetonitrile (ACN) containing 0.1% formic acid. After the removal of protein by centrifugation at 1,640 g (3,000 rpm) for 10 minutes at 4° C., the supernatants were transferred to an HPLC autosampler plate. The remaining of TA (expressed as the peak area ratio of test article to IS) was determined by LC-MS/MS. CYP enzyme activities of the HLM were verified in parallel by determining the formation of individual CYP probe metabolites by LC-MS/MS using standard curves. The experimental conditions for CYP reaction phenotyping and sample analysis are summarized in Table 12 and Table 13.

Data Analysis

The percent remaining of TA was calculated using the following equation:

% Remaining of TA=100×Aₜ/A₀

Where, At and A0 are the peak area ratio of TA to IS at time t and at time zero, respectively.

Results

The percent remaining of PB with pooled HLM (0.5 mg protein/mL) in the presence and absence of CYP-selective inhibitors are summarized in Table 14 and Table 15. The percent remaining of TUDCA with pooled HLM (0.5 mg protein/mL) in the presence and absence of CYP-selective inhibitors are summarized in Table 16 and Table 17. CYP enzyme activities of the HLM were verified in parallel by determining the formation of CYP probe metabolites using LC-MS/MS, and the results are summarized in Table 18 and Table 19.

TABLE 11
CYP-selective Chemical Inhibitors

| CYP | Chemical Inhibitor | Vendor | Catalog # |
|---|---|---|---|
| CYP1A2 | Furafylline | MilliporeSigma | F124 |
| CYP2B6 | Thio-TEPA | MilliporeSigma | T6089 |
| CYP2C8 | Montelukast | Bosche | M2936 |
| | | Scientific | |
| CYP2C9 | Sulfaphenazole | MilliporeSigma | S0758 |
| CYP2C19 | (+)-N-3-benzylnirvanol (N3B) | MilliporeSigma | B8686 |
| CYP2D6 | Quinidine | MilliporeSigma | Q3625 |
| CYP3A | Ketoconazole | MilliporeSigma | K1003 |

TABLE 12
Incubation Conditions for CYP Reaction Using HLM

| | | Concen- | Pre- | |
|---|---|---|---|---|
| | | tration | incubation | Inhibition |
| CYP | Inhibitor | (μM) | (15 min) | Condition |
| CYP1A2 | Furafylline | 10 | Yes | Irreversible |
| CYP2B6 | Thio-TEPA | 10 | No | Reversible |
| CYP2C8 | Montelukast | 5 | No | Reversible |
| CYP2C9 | Sulfaphenazole | 5 | No | Reversible |
| CYP2C19 | N3B | 5 | No | Reversible |
| CYP2D6 | Quinidine | 1 | No | Reversible |
| CYP3A | Ketoconazole | 1 | No | Reversible |
| Negative | −inhibitor, | N/A | Yes | Irreversible |
| Control-1 | −NADPH | | | |
| Negative | −inhibitor, | N/A | No | Reversible |
| Control-2 | −NADPH | | | |
| Maximum | −inhibitor, | N/A | Yes | Irreversible |
| Metabolism-1 | +NADPH | | | |
| Maximum | −inhibitor, | N/A | No | Reversible |
| Metabolism -2 | +NADPH | | | |

TABLE 13
CYP Reaction Using HLM and Sample Analysis

| | |
|---|---|
| Incubation | TA: 5 μM; HLM: 0.5 mg protein/mL; |
| Composition | Phosphate buffer: 100 mM, pH 7.4; MgCl₂: 5 mM |
| Equilibration | 37° C. for 5 min |
| NADPH | 1 mM |
| Pre-incubation | 37° C. for 15 min for irreversible incubation |
| Incubation Time | 37° C. for 0, 5, 10, 20, 30, and 60 min (n = 3) |
| Reaction | ACN containing 0.1% formic acid and IS (1:2, v/v) |
| Termination | |
| Sample Treatment | Centrifugation at 1,640 g (3,000 rpm) for 10 min |
| | at 4° C. |
| Sample Analysis | LC-MS/MS |

TABLE 14
% Remaining of PB in HLM (Irreversible Incubation Condition)

| | | % Remaining of TA ^{a} | | | | | | |
|---|---|---|---|---|---|---|---|---|
| Reaction | Treatment | (n = 3) | 0 min | 5 min | 10 min | 20 min | 30 min | 60 min |
| Negative | −NADPH | Average | 100 | 109 | 114 | 113 | 112 | 115 |
| Control-1 | −Inhibitor | SD | 7.86 | 5.44 | 4.63 | 1.36 | 2.03 | 2.65 |
| Maximum | +NADPH | Average | 100 | 107 | 118 | 113 | 110 | 116 |
| Metabolism-1 | −Inhibitor | SD | 8.72 | 5.95 | 6.28 | 2.65 | 6.87 | 5.15 |
| CYP1A2 | +NADPH | Average | 100 | 116 | 131 | 125 | 127 | 128 |
| | +Furafylline | SD | 8.45 | 1.19 | 2.43 | 8.17 | 0.915 | 5.14 |

^{a} The % remaining (n = 3) was calculated from the peak area ratio of TA to IS by LC-MS/MS.
SD: standard deviation.

TABLE 15
% Remaining of PB in HLM (Reversible Incubation Condition)

| | | % Remaining of TA ^{a} | | | | | | |
|---|---|---|---|---|---|---|---|---|
| | | | 0 | 5 | 10 | 20 | 30 | 60 |
| Reaction | Treatment | n = 3 | min | min | min | min | min | min |
| Negative | −NADPH | Average | 100 | 99.5 | 99.7 | 99.7 | 102 | 102 |
| Control-2 | −Inhibitor | SD | 4.31 | 2.14 | 2.46 | 5.53 | 1.18 | 3.32 |
| Maximum | +NADPH | Average | 100 | 97.9 | 97.7 | 98.9 | 98.2 | 95.0 |
| Metabolism-2 | −Inhibitor | SD | 1.02 | 6.93 | 4.90 | 1.56 | 2.09 | 4.38 |
| CYP2B6 | +NADPH | Average | 100 | 106 | 99.9 | 100 | 101 | 96.9 |
| | +Thio-TEPA | SD | 1.75 | 2.57 | 0.765 | 1.15 | 6.99 | 2.76 |
| CYP2C8 | +NADPH | Average | 100 | 99.7 | 104 | 102 | 102 | 99.0 |
| | +Montelukast | SD | 4.50 | 2.48 | 1.03 | 1.94 | 3.78 | 5.57 |
| CYP2C9 | +NADPH | Average | 100 | 103 | 101 | 98.5 | 99.6 | 97.6 |
| | +Sulfaphenazole | SD | 3.27 | 1.21 | 4.02 | 7.41 | 3.48 | 6.46 |
| CVP2C19 | +NADPH | Average | 100 | 101 | 102 | 101 | 101 | 101 |
| | +N3B | SD | 4.47 | 2.21 | 1.58 | 3.27 | 3.53 | 3.27 |
| CYP2D6 | +NADPH | Average | 100 | 103 | 103 | 97.5 | 98.2 | 98.8 |
| | +Quinidine | SD | 3.95 | 6.59 | 0.883 | 0.520 | 4.04 | 3.29 |
| CYP3A | +NADPH | Average | 100 | 100 | 101 | 100 | 98.2 | 90.6 |
| | +Ketoconazole | SD | 2.07 | 1.32 | 1.85 | 2.71 | 3.48 | 6.69 |

^{a} The % remaining (n = 3) was calculated from the peak area ratio of TA to IS by LC-MS/MS.
SD: standard deviation.

TABLE 16
% Remaining of TUDCA in HLM (Irreversible Incubation Condition)

| | | % Remaining of TA ^{a} | | | | | | |
|---|---|---|---|---|---|---|---|---|
| Reaction | Treatment | (n = 3) | 0 min | 5 min | 10 min | 20 min | 30 min | 60 min |
| Negative | −NADPH | Average | 100 | 117 | 129 | 119 | 130 | 132 |
| Control-1 | −Inhibitor | SD | 3.24 | 2.81 | 4.15 | 6.58 | 5.10 | 5.33 |
| Maximum | +NADPH | Average | 100 | 112 | 126 | 123 | 121 | 121 |
| Metabolism-1 | −Inhibitor | SD | 11.9 | 16.0 | 14.5 | 5.26 | 8.75 | 6.47 |
| CYP1A2 | +NADPH | Average | 100 | 123 | 123 | 121 | 120 | 128 |
| | +Furafylline | SD | 18.5 | 6.39 | 5.51 | 12.0 | 8.12 | 9.29 |

^{a} The % remaining (n = 3) was calculated from the peak area ratio of TA to IS by LC-MS/MS.
SD: standard deviation.

TABLE 17
% Remaining of TUDCA in HLM (Reversible Incubation Condition)

| | | % Remaining of TA ^{a} | | | | | | |
|---|---|---|---|---|---|---|---|---|
| | | | 0 | 5 | 10 | 20 | 30 | 60 |
| Reaction | Treatment | n = 3 | min | min | min | min | min | min |
| Negative | −NADPH | Average | 100 | 104 | 104 | 105 | 106 | 106 |
| Control-2 | −Inhibitor | SD | 2.22 | 2.42 | 2.38 | 4.15 | 1.38 | 2.72 |
| Maximum | +NADPH | Average | 100 | 103 | 103 | 103 | 102 | 96.9 |
| Metabolism-2 | −Inhibitor | SD | 1.22 | 4.71 | 2.89 | 0.956 | 3.23 | 3.47 |
| CYP2B6 | +NADPH | Average | 100 | 112 | 112 | 122 | 116 | 102 |
| | +Thio-TEPA | SD | 1.00 | 9.98 | 11.1 | 6.68 | 4.80 | 2.47 |
| CYP2C8 | +NADPH | Average | 100 | 101 | 103 | 95.5 | 105 | 97.7 |
| | +Montelukast | SD | 5.84 | 2.86 | 4.51 | 9.57 | 7.27 | 1.30 |
| CYP2C9 | +NADPH | Average | 100 | 102 | 96.9 | 92.8 | 95.0 | 95.3 |
| | +Sulfaphenazole | SD | 5.47 | 3.96 | 9.82 | 3.48 | 5.76 | 21.2 |
| CYP2C19 | +NADPH | Average | 100 | 106 | 93.0 | 93.1 | 92.1 | 94.5 |
| | +N3B | SD | 10.1 | 10.6 | 6.06 | 5.41 | 6.89 | 13.1 |
| CYP2D6 | +NADPH | Average | 100 | 101 | 110 | 99.7 | 101 | 87.2 |
| | +Quinidine | SD | 1.95 | 6.46 | 6.30 | 11.8 | 10.4 | 10.2 |
| CYP3A | +NADPH | Average | 100 | 103 | 110 | 108 | 94.1 | 105 |
| | +Ketoconazole | SD | 11.5 | 4.45 | 7.55 | 7.19 | 8.69 | 12.5 |

^{a} The % remaining (n = 3) was calculated from the peak area ratio of TA to IS by LC-MS/MS.
SD: standard deviation.

TABLE 18
Formation of CYP Probe Metabolites in HLM

| | | | | Measured | | |
|---|---|---|---|---|---|---|
| | | | | Metabolite | Formation | Formation |
| | | | | (μM) | Rate | Rate |
| Probe | Metabolite | Treatment | Reaction | (Average, n = 2)ª | (Average, n = 2)^{b} | Ratio^{c} |
| Phenacerin | Acetaminophen | −NADPH | Negative | 0 | 0 | — |
| | | −Inhibitor | Control | | | |
| | | +NADPH | Maximum | 2.98 | 298 | 23 |
| | | −Inhibitor | Metabolism | | | |
| | | +NADPH | CTP1A2 | 0.130 | 13.0 | |
| | | +Furafylline | | | | |
| Bupropion | OH | −NADPH | Negative | 0 | 0 | — |
| (100 μM) | Bupropion | −Inhibitor | Control | | | |
| | | +NADPH | Maximum | 1.33 | 133 | 2.0 |
| | | −Inhibitor | Metabolism | | | |
| | | +NADPH | CYP2B6 | 0.679 | 67.9 | |
| | | +Thio-TEPA | | | | |
| Amodiaquine | Desethylamodiaquine | −NADPH | Negative | 0 | 0 | — |
| (1 μM) | | −Inhibitor | Control | | | |
| | | +NADPH | Maximum | 0.977 | 97.7 | 4.2 |
| | | −Inhibitor | Metabolism | | | |
| | | +NADPH | CYP2C8 | 0.234 | 23.4 | |
| | | +Montelukast | | | | |
| Diclofenac | 4′-OH | −NADPH | Negative | 0 | 0 | — |
| (4 μM) | Diclofenac | −Inhibitor | Control | | | |
| | | +NADPH | Maximum | 4.83 | 483 | 7.7 |
| | | −Inhibitor | Metabolism | | | |
| | | +NADPH | CYP2C9 | 0.630 | 6.30 | |
| | | +Sulfaphenazole | | | | |
| S-Mephenytoin | 4′-OH | −NADPH | Negative | 0 | 0 | — |
| (25 μM) | Mephenytoin | −Inhibitor | Control | | | |
| | | +NADPH | Maximum | 0.279 | 27.9 | 22 |
| | | −Inhibitor | Metabolism | | | |
| | | +NADPH | CYP2C19 | 0.0130 | 1.30 | |
| | | +N3B | | | | |

^{a}The concentrations (average, n = 2) of CYP probe metabolites were measured by LC-MS/MS using standard curves.
^{b}The formation rates of CYP probe metabolites are expressed as pmol/min/mg protein. When a probe metabolite was not detectable, the rate of formation is reported as zero.
^{c}Formation rate ratio = [Formation Rate_{(+NADPH-Inhibitor)} − Formation Rate_{(-NADPH-Inhibitor)}]/[Formation Rate_{(+NADPH+Inhibitor)} − Formation Rate_{(-NADPH-Inhibitor)}].
QC acceptance criterion: formation rate ratio ≥2.
SD: standard desnation

TABLE 19
Formation of CYP Probe Metabolites in HLM (Continued)

| | | | | Measured | Formation | Formation |
|---|---|---|---|---|---|---|
| | | | | Metabolite | Rate | Rate |
| Probe | Metabolite | Treatment | Reaction | (Average, n = 2)^{a} | (Average, n = 2)^{b} | Ratio^{c} |
| Bufuralol | 1′-OH | −NADPH | Negative | 0 | 0 | — |
| (7 μM) | Bufuralol | −Inhibitor | Control | | | |
| | | +NADPH | Maximum | 0.470 | 47.0 | 5.9 |
| | | −Inhibitor | Metabolism | | | |
| | | +NADPH | CYP2D6 | 0.0791 | 7.91 | |
| | | +Quinidine | | | | |
| Midazolam | 1′-OH | −NADPH | Negative | 0 | 0 | — |
| (2 μM) | Midazolam | −Inhibitor | Control | | | |
| | | +NADPH | Maximum | 2.18 | 218 | 21 |
| | | −Inhibitor | Metabolism | | | |
| | | +NADPH | CYP3A | 0.104 | 10.4 | |
| | | +Ketoconazole | | | | |
| Testosterone | 6β-OH | −NADPH | Negative | 0 | 0 | — |
| (70 μM) | Testosterone | −Inhibitor | Control | | | |
| | | +NADPH | Maximum | 22.2 | 2215 | 22 |
| | | −Inhibitor | Metabolism | | | |
| | | +NADPH | CYP3A | 1.13 | 113 | |
| | | +Ketoconazole | | | | |

### Example 4—Cytochrome P450 Reaction Phenotyping of Sodium Phenylbutyrate and Tauroursodeoxycholic Acid Using Human Recombinant Cyp Enzymes

Cytochrome P450 (CYP) reaction phenotyping of sodium phenylbutyrate (PB) and tauroursodeoxycholic acid (TUDCA) was evaluated using human recombinant CYP enzymes (hrCYPs). PB and TUDCA (5 μM each) were co-incubated with individual hrCYPs (20 pmol CYP/mL) in phosphate buffer (100 mM, pH 7.4) containing MgCl2 (5 mM) and NADPH (1 mM). The amounts of PB and TUDCA remaining after a period of incubation (0, 5, 10, 20, 30, and 60 minutes) were measured by LC-MS/MS.

PA and TUDCA, at one concentration each (5 μM in the final incubation), were co-incubated with an individual hrCYP (20 pmol CYP/mL) or CYP control (negative control without CYP enzymes, 0.1 mg protein/mL) in phosphate buffer (100 mM, pH 7.4) containing MgCl2 (5 mM) and NADPH (1 mM). The incubation mixture without NADPH was equilibrated in a shaking water bath at 37° C. for 5 minutes. The reaction was initiated by the addition of NADPH, followed by incubation at 37° C. Aliquots (100 μL) of the incubation solutions were sampled at 0, 5, 10, 20, 30, and 60 minutes. The reaction was terminated by the addition of ice-cold acetonitrile (ACN) containing 0.1% formic acid. After the removal of protein by centrifugation at 1,640 g (3,000 rpm) for 10 minutes at 4° C., the supernatants were transferred to an HPLC autosampler plate and stored at −20° C. until analysis. The remaining test article (TA) (expressed as the peak area ratio of TA to IS) was determined by LC-MS/MS. hrCYP activities were verified in parallel by determining the formation of CYP probe metabolites after 20 minutes of incubation with individual CYP probe substrates by LC-MS/MS using standard curves. The experimental conditions for CYP reaction phenotyping and sample analysis are summarized in Table 20.

TABLE 20
CYP Reaction Using hrCYPs and Sample Analysis

| | |
|---|---|
| Incubation | TA: 5 μM; hrCYPs: 20 pmol CYP/mL; |
| Composition | Phosphate buffer: 100 mM, pH 7.4; MgCl₂: 5 mM |
| Equilibration | 37° C. for 5 min |
| NADPH | 1 mM |
| Incubation Time | 37° C. for 0, 5, 10, 20, 30, and 60 min (n = 3) |
| Reaction | ACN containing 0.1% formic acid and IS (1:2, v/v) |
| Termination | |
| Sample Treatment | Centrifugation at 1,640 g (3.000 rpm) for 10 |
| | minutes at 4° C. |
| Sample Analysis | LC-MS/MS |

The percent remaining of TA was calculated using the following equation:

% Remaining of TA=100×Aₜ/A₀

Where, Aₜ is the peak area ratio of TA to internal standard (IS) at time t, A₀ is the average peak area ratio of TA to IS at time zero. The percent remaining values of PB and TUDCA in hrCYPs (20 pmol CYP/mL) are summarized in Table 21 and Table 22, respectively. hrCYP activities were verified in parallel by determining the formation of CYP probe metabolites using LC-MS/MS, and the results are summarized in Table 23.

TABLE 21
% Remaining of PB in hrCYPs

| | % Remaining of TA ª | | | | | | |
|---|---|---|---|---|---|---|---|
| hrCYP | (n = 3) | 0 min | 5 min | 10 min | 20 min | 30 min | 60 min |
| CYP1A2 | Average | 100 | 98.3 | 103 | 99.9 | 101 | 95.1 |
| | SD | 9.77 | 10.0 | 8.53 | 9.84 | 11.1 | 8.80 |
| CYP2B6 | Average | 100 | 98.6 | 99.6 | 97.3 | 99.7 | 99.3 |
| | SD | 3.36 | 0.855 | 2.18 | 1.85 | 0.826 | 1.94 |
| CYP2C8 | Average | 100 | 100 | 101 | 102 | 103 | 101 |
| | SD | 0.675 | 2.34 | 6.52 | 2.26 | 3.88 | 3.07 |
| CYP2C9 | Average | 100 | 101 | 101 | 99.5 | 101 | 98.0 |
| | SD | 1.76 | 2.87 | 1.75 | 2.25 | 3.50 | 0.771 |
| CYP2C19 | Average | 100 | 97.4 | 101 | 99.7 | 97.5 | 99.8 |
| | SD | 0.484 | 1.21 | 1.22 | 2.98 | 2.51 | 2.13 |
| CYP2D6 | Average | 100 | 99.9 | 96.7 | 97.6 | 101 | 100 |
| | SD | 1.58 | 2.14 | 2.00 | 0.373 | 1.98 | 3.42 |
| CYP3A4 | Average | 100 | 100 | 97.9 | 100 | 98.1 | 102 |
| | SD | 2.11 | 3.75 | 2.50 | 0.743 | 4.66 | 2.39 |
| CYP | Average | 100 | 101 | 98.2 | 95.4 | 93.6 | 101 |
| Control | SD | 0.0978 | 0.341 | 1.74 | 2.54 | 2.50 | 3.38 |

^{a} The % remaining of TA was calculated from the peak area ratio of TA to IS by LC-MS/MS.
SD: standard deviation.

TABLE 22
% Remaining of TUDCA in hrCYPs

| | % Remaining of TA ^{a} | | | | | | |
|---|---|---|---|---|---|---|---|
| hrCYP | (n = 3) | 0 min | 5 min | 10 min | 20 min | 30 min | 60 min |
| CYP1A2 | Average | 100 | 98.0 | 101 | 99.9 | 103 | 90.5 |
| | SD | 8.14 | 7.44 | 8.79 | 11.5 | 10.3 | 6.84 |
| CYP2B6 | Average | 100 | 97.6 | 97.0 | 99.3 | 99.7 | 101 |
| | SD | 4.16 | 1.01 | 0.586 | 1.35 | 0.825 | 1.48 |
| CYP2C8 | Average | 100 | 97.1 | 99.3 | 101 | 101 | 99.8 |
| | SD | 3.08 | 1.86 | 3.45 | 4.62 | 1.04 | 1.26 |
| CYP2C9 | Average | 100 | 96.0 | 98.8 | 98.5 | 97.8 | 90.3 |
| | SD | 3.43 | 1.17 | 2.23 | 1.21 | 2.06 | 0.716 |
| CYP2C19 | Average | 100 | 96.1 | 98.7 | 94.8 | 95.1 | 91.4 |
| | SD | 0.845 | 1.65 | 0.860 | 2.84 | 1.26 | 1.80 |
| CYP2D6 | Average | 100 | 98.4 | 97.8 | 97.0 | 99.2 | 98.6 |
| | SD | 2.33 | 1.71 | 3.92 | 1.35 | 0.335 | 2.75 |
| CYP3A4 | Average | 100 | 97.1 | 92.1 | 91.4 | 90.9 | 91.1 |
| | SD | 1.07 | 1.88 | 2.87 | 1.16 | 2.68 | 2.52 |
| CYP | Average | 100 | 99.4 | 97.9 | 95.9 | 93.7 | 101 |
| Control | SD | 4.52 | 4.15 | 2.52 | 3.89 | 4.05 | 3.60 |

TABLE 23
Formation of CYP Probe Metabolites in hrCYPs

| | | | Measured | | |
|---|---|---|---|---|---|
| Probe | | | Metabolite | Formation | Formation |
| Substrate | Metabolite | Treatment | (μM)^{a} | Rate^{b} | Rate Ratio^{c} |
| Phenacetin | Acetaminophen | hrCYP1A2 | 4.03 | 2239 | >>2 |
| (50 μM) | | CYP Control | 0 | 0 | |
| Bupropion | OH Bupropion | hrCYP2B6 | 4.28 | 1162 | >>2 |
| (50 μM) | | CYP Control | 0 | 0 | |
| Amodiaquine | Desethylamodiaquine | hrCYP2C8 | 0.801 | 257 | >>2 |
| (2 μM) | | CYP Control | 0 | 0 | |
| Diclofenac | 4′-OH Diclofenac | hrCYP2C9 | 1.56 | 845 | >>2 |
| (6 μM) | | CYP Control | 0 | 0 | |
| S-mephenytoin | 4′-OH Mephenytoin | hrCYP2C19 | 0.454 | 324 | >>2 |
| (20 μM) | | CYP Control | 0 | 0 | |
| Bufuralol | 1′-OH Bufuralol | hrCYP2D6 | 3.77 | 1024 | >>2 |
| (7 μM) | | CYP Control | 0 | 0 | |
| Midazolam | 1′-OH Midazolam | hrCYP3A4 | 1.46 | 456 | >>2 |
| (2 μM) | | CYP Control | 0 | 0 | |
| Testosterone | 6β-OH Testosterone | hrCYP3A4 | 22.2 | 6922 | >>2 |
| (50 μM) | | CYP Control | 0 | 0 | |

^{a}The concentrations (average, n = 2) of CYP probe metabolites were measured by LC-MS/MS.
^{b}The formation rates of CYP probe metabolites were normalized and expressed as pmol metabolite/min/mg Supersome protein. When a probe metabolite was not detectable, the rate of formation is reported as zero.
^{c}Formation rate ratio = Formation rate_{(hrCYP)}/Formation rate_{(Negative Control)}. QC acceptance criterion: formation rate ratio ≥ 2.

After 60 minutes of incubation with hrCYPs, greater than 90% of PB and TUDCA remained (<10% disappearance) in the presence of hrCYP1A2, hrCYP2B6, hrCYP2C8, hrCYP2C9, hrCYP2C19, hrCYP2D6, and hrCYP3A4. The results suggest that neither PB nor TUDCA is metabolized by the major hepatic CYPs.

### Example 5—Evaluation of the Potential for Time-Dependent Inhibition of Cytochrome P450 Enzymes in Human Liver Microsomes by Sodium Phenylbutyrate and Tauroursodeoxycholic Acid

The potential for time-dependent inhibition (TDI) of cytochrome P450 (CYP) enzymes in human liver microsomes (HLM) by sodium phenylbutyrate (PB) and tauroursodeoxycholic acid (TUDCA) was evaluated. PB and TUDCA at eight concentrations were pre-incubated (co-incubation with PB and TUDCA) with pooled HLM (0.25 mg protein/mL) for 30 minutes in the presence and absence of NADPH, followed by a CYP enzyme activity assay with an individual CYP probe substrate and quantification by measuring the formation of each CYP probe metabolite by LC-MS/MS.

The results showed that the difference in CYP inhibition by PB and TUDCA at all concentrations of PB and TUDCA, with 30-minute pre-incubation in the absence and presence of NADPH, was less than 20% for all of the seven major hepatic CYPs tested in this study. This suggests that the IC50 shift would be <1.5 (the threshold for TDI), although the individual IC50 and IC50 shift values of PB and TUDCA could not be calculated due to co-incubation of PB and TUDCA. The results suggest that TDI of CYPs by PB and TUDCA is unlikely.

CYP TDI was evaluated by a 30-minute pre-incubation of the TA with HLM in the presence and absence of NADPH followed by the CYP enzyme activity assay. The CYP reaction was performed in an incubation volume of 200 μL. Briefly, PB (0-7400 μM, 50×C_{max,u}=7400 μM, 0.1×I_{gut}=6444 μM) and TUDCA (0-1600 μM, 50×C_{max,u}=81 μM, 0.1×I_{gut}=1600 μM) at eight concentrations per test article were pre-incubated (co-incubation of PB and TUDCA) at 37° C. for 30 minutes with HLM (0.25 mg protein/mL) in phosphate buffer (100 mM, pH 7.4) containing MgCl2 (5 mM) in the presence (irreversible incubation conditions) and absence (reversible incubation conditions) of NADPH (1 mM). The total organic solvent content in the final incubation was less than 1% (DMSO ≤0.1%, other organic solvent ≤1%). The CYP reactions were initiated by adding an individual CYP probe substrate with (when NADPH was not added in the pre-incubation step) or without (when NADPH was added in the pre-incubation step) the addition of NADPH (1 mM). The reaction mixture was incubated at 37° C. for 10-30 minutes depending on the individual CYP isoform (Table 24). The reaction was terminated with ice-cold acetonitrile (ACN) containing an internal standard (IS, stable isotope-labeled CYP probe metabolite). Negative (vehicle) controls were conducted using the incubation mixture without the TA. Positive controls were performed in parallel using known CYP time-dependent inhibitors. After the removal of protein by centrifugation at 1,640 g (3,000 rpm) for 10 minutes at 4° C., the supernatants were transferred to an HPLC autosampler plate. The formation of individual CYP probe metabolites was determined by LC-MS/MS. The experimental conditions for CYP reaction and sample analysis are summarized in Table 24.

The percent of control enzyme activity was calculated using the following equation:

% of control enzyme activity=100×(enzyme activity in the presence of TA/enzyme activity in the absence of TA)

The enzyme activity was expressed as the peak area ratio of CYP probe metabolite to IS, measured by LC-MS/MS. The IC50 value was estimated by fitting the experimental data (percent enzyme activity of control vs. log [inhibitor concentration] to a sigmoidal model, followed by non-linear regression analysis using GraphPad Prism (Version 5.0 or higher, GraphPad Software, San Diego, Calif., USA).

TABLE 24
CYP Reaction and Sample Analysis

| | |
|---|---|
| Incubation | PB: 0-7400 μM; TUDCA: 0-1600 μM; HLM: 0.25 mg |
| Composition | protein/mL; Phosphate buffer: 100 mM, pH 7.4; MgCl₂: |
| | 5 mM; Probe substrate: ~Kₘ |
| NADPH | 1 mM |
| Pre-incubation | 37° C. for 30 min with and without NADPH |
| Incubation Time | 37° C. for 10 min (CYP3A). 30 min (CYP2C19), or 20 min (all other CYPs) |
| for Activity Assay | |
| Reaction Termination | ACN containing IS (1:2, v/v) |
| Sample Treatment | Centrifugation at 1,640 g (3,000 rpm) for 10 min at 4° C. |
| Sample Analysis | LC-MS/MS |

The IC50 shift between reversible (30 minutes of pre-incubation without NADPH) or irreversible (30 minutes of pre-incubation with NADPH) incubation conditions is an index of TDI potential: the threshold for a positive result is IC50 shift >1.5.

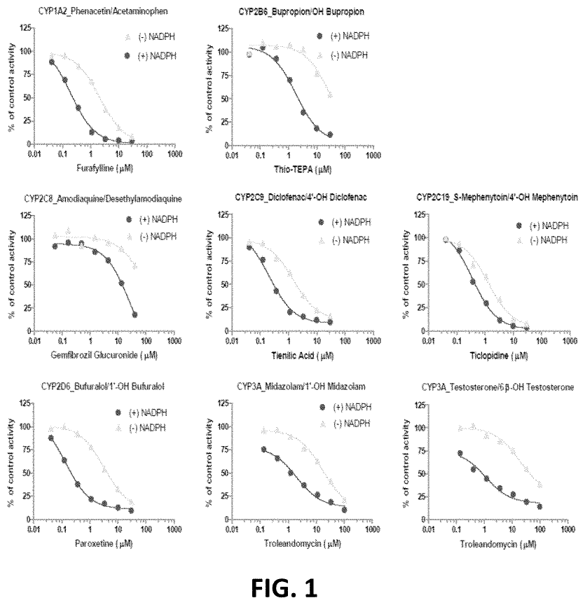

The potential for CYP TDI in HLM by PB and TUDCA is summarized in Table 25. The inhibition of CYP activities by positive time-dependent inhibitors is summarized in Tables 26-27 and FIG. 1.

TABLE 25
Potential for CYP TDI in HLM by PB and TUDCA

| | Pre- | % of Control Enzyme Activity (n = 3)^{a} | | | | | | | | | |
|---|---|---|---|---|---|---|---|---|---|---|---|
| | incubation | PB (μM) | | 10.2 | 30.5 | 91.4 | 274 | 822 | 2467 | 7400 | |
| CYP | (30 min) | TUDCA (μM) | 0 | 2.19 | 6.58 | 19.8 | 59.3 | 178 | 533 | 1600 | TDI^{b} |
| CYP1A2 | −NADPH | Average | 100 | 103 | 105 | 102 | 103 | 101 | 87.7 | 7.13 | Unlikely |
| | | SD | 2.68 | 2.88 | 3.63 | 3.35 | 1.23 | 6.49 | 0.913 | 1.23 | |
| | +NADPH | Average | 100 | 104 | 102 | 99.8 | 101 | 95.3 | 81.3 | 8.79 | |
| | | SD | 5.44 | 5.80 | 7.52 | 4.57 | 5.22 | 3.80 | 1.78 | 3.45 | |
| CYP2B6 | −NADPH | Average | 100 | 96.0 | 100 | 98.5 | 92.3 | 76.2 | 41.7 | 9.54 | Unlikely |
| | | SD | 5.09 | 2.96 | 1.53 | 5.44 | 2.92 | 8.08 | 1.75 | 1.06 | |
| | +NADPH | Average | 100 | 102 | 109 | 106 | 103 | 88.0 | 49.5 | 9.93 | |
| | | SD | 11.0 | 1.30 | 6.48 | 1.65 | 6.10 | 5.57 | 7.41 | 1.66 | |
| CYP2C8 | −NADPH | Average | 100 | 98.7 | 97.2 | 94.3 | 97.1 | 83.9 | 73.8 | 42.0 | Unlikely |
| | | SD | 2.19 | 2.04 | 8.69 | 0.550 | 5.53 | 3.86 | 0.546 | 7.07 | |
| | +NADPH | Average | 100 | 98.6 | 99.4 | 91.5 | 90.6 | 88.2 | 73.6 | 40.8 | |
| | | SD | 6.93 | 11.8 | 10.0 | 3.86 | 4.56 | 6.18 | 5.46 | 3.17 | |
| CYP2C9 | −NADPH | Average | 100 | 98.3 | 104 | 97.6 | 89.6 | 83.5 | 61.4 | 39.3 | Unlikely |
| | | SD | 4.94 | 5.98 | 0.876 | 4.91 | 2.92 | 5.30 | 3.37 | 3.00 | |
| | +NADPH | Average | 100 | 96.4 | 98.4 | 90.5 | 88.2 | 74.0 | 59.0 | 36.3 | |
| | | SD | 0.947 | 2.57 | 4.57 | 6.60 | 2.34 | 3.94 | 2.00 | 1.44 | |
| CYP2C19 | −NADPH | Average | 100 | 98.4 | 100 | 92.7 | 104 | 93.2 | 84.0 | 56.1 | Unlikely |
| | | SD | 0.614 | 1.45 | 2.77 | 6.56 | 8.43 | 5.87 | 5.67 | 2.42 | |
| | +NADPH | Average | 100 | 89.7 | 95.6 | 90.2 | 84.1 | 82.2 | 76.6 | 53.2 | |
| | | SD | 9.51 | 8.86 | 3.73 | 10.8 | 7.09 | 6.82 | 1.86 | 5.73 | |
| CYP2D6 | −NADPH | Average | 100 | 108 | 110 | 113 | 115 | 115 | 113 | 61.3 | Unlikely |
| | | SD | 2.17 | 4.08 | 3.15 | 1.58 | 1.61 | 5.30 | 3.87 | 4.69 | |
| | +NADPH | Average | 100 | 109 | 113 | 112 | 116 | 115 | 117 | 75.7 | |
| | | SD | 6.19 | 6.95 | 5.21 | 2.85 | 1.76 | 3.61 | 4.06 | 18.0 | |
| CYP3A | −NADPH | Average | 100 | 93.7 | 92.2 | 93.6 | 89.2 | 82.1 | 70.2 | 24.3 | Unlikely |
| (Midazolam) | | SD | 8.41 | 3.57 | 2.22 | 1.13 | 1.12 | 1.97 | 0.818 | 5.69 | |
| | +NADPH | Average | 100 | 95.2 | 94.5 | 92.9 | 89.3 | 82.1 | 63.9 | 15.4 | |
| | | SD | 4.57 | 4.37 | 1.14 | 0.747 | 2.32 | 1.72 | 1.49 | 5.66 | |
| CYP3A | −NADPH | Average | 100 | 97.6 | 102 | 97.0 | 104 | 96.8 | 82.8 | 35.4 | Unlikely |
| (Testosterone) | | SD | 2.90 | 4.41 | 3.54 | 1.84 | 3.28 | 0.692 | 4.88 | 6.43 | |
| | +NADPH | Average | 100 | 102 | 98.8 | 103 | 101 | 89.8 | 75.8 | 17.7 | |
| | | SD | 3.24 | 0.628 | 2.47 | 4.85 | 4.67 | 1.75 | 1.87 | 1.93 | |

^{a}Percent of control enzyme activity = 100 × (Enzyme activity in the presence of TA/Enzyme activity in the absence of TA). Enzyme activity was calculated from the peak area ratio of CYP probe metabolite to IS by LC-MS/MS.
^{b}Although the individual IC50 and IC50 shift values of PB and TUDCA could not be calculated due to co-incubation of PB and TUDCA, the difference in CYP inhibition by PB and TUDCA at the highest concentrations (7400 and 1600 μM for PB and TUDCA, respectively) with 30-min pre-incubation in the absence and presence of NADPH was less than 20%, suggesting that the IC50 shift would be <1.5 and the conclusion would be that TDI is unlikely.
SD: standard deviation.

TABLE 26
IC50 Shift for CYP TDI in HLM by Positive Time-dependent Inhibitors

| | Pre- | | | | | | | | | | | |
|---|---|---|---|---|---|---|---|---|---|---|---|---|
| | incubation | | | | | | | | | | IC₅₀ | IC₅₀ |
| CYP | (30 min) | | % of Control Enzyme Activity (n = 3)^{a} | | | | | | | | (μM) | Shift^{b} |
| CYP1A2 | Furafylline | — | 0 | 0.0412 | 0.123 | 0.370 | 1.11 | 3.33 | 10.0 | 30.0 | — | — |
| | | | μM | μM | μM | μM | μM | μM | μM | μM | | |
| | −NADPH | Average | 100 | 95.6 | 95.6 | 84.1 | 67.8 | 37.2 | 18.0 | 8.21 | 2.14 | 9.17 |
| | | SD | 9.24 | 5.37 | 2.16 | 0.699 | 3.68 | 0.621 | 0.975 | 0.452 | | |
| | +NADPH | Average | 100 | 88.6 | 69.3 | 39.1 | 12.9 | 5.56 | 4.09 | 3.75 | 0.234 | |
| | | SD | 2.40 | 2.81 | 4.57 | 2.30 | 1.82 | 0.454 | 0.0710 | 0.451 | | |
| CYP2B6 | Thio-TEPA | — | 0 | 0.0412 | 0.123 | 0.370 | 1.11 | 3.33 | 10.0 | 30.0 | — | — |
| | | | μM | μM | μM | μM | μM | μM | μM | μM | | |
| | −NADPH | Average | 100 | 99.3 | 110 | 106 | 108 | 103 | 76.7 | 54.8 | >30.0 | >13.3 |
| | | SD | 1.84 | 3.30 | 5.42 | 9.28 | 11.7 | 5.06 | 3.28 | 5.49 | | |
| | +NADPH | Average | 100 | 97.3 | 105 | 93.0 | 69.9 | 35.5 | 18.3 | 11.6 | 2.25 | |
| | | SD | 10.9 | 0.528 | 7.64 | 3.63 | 6.29 | 1.68 | 1.31 | 1.35 | | |
| CYP2C8 | Gemfibrozil | — | 0 | 0.0549 | 0.165 | 0.494 | 1.48 | 4.44 | 13.3 | 40.0 | — | — |
| | glucuronide | | μM | μM | μM | μM | μM | μM | μM | μM | | |
| | −NADPH | Average | 100 | 104 | 109 | 92.5 | 101 | 96.1 | 89.4 | 71.3 | >40.0 | >2.83 |
| | | SD | 1.50 | 1.39 | 1.88 | 6.28 | 4.47 | 6.20 | 4.20 | 3.81 | | |
| | +NADPH | Average | 100 | 91.8 | 96.0 | 94.9 | 85.8 | 76.6 | 51.9 | 17.8 | 14.2 | |
| | | SD | ND | 3.10 | 8.63 | 8.19 | 5.82 | 4.63 | 2.11 | 1.12 | | |
| CYP2C9 | Tienilic acid | — | 0 | 0.0412 | 0.123 | 0.370 | 1.11 | 3.33 | 10.0 | 30.0 | — | — |
| | | | μM | μM | μM | μM | μM | μM | μM | μM | | |
| | −NADPH | Average | 100 | 95.2 | 94.3 | 77.8 | 62.4 | 38.4 | 22.2 | 16.2 | 1.85 | 6.24 |
| | | SD | 3.52 | 1.76 | 1.28 | 1.52 | 1.72 | 2.44 | 0.297 | 0.707 | | |
| | +NADPH | Average | 100 | 89.7 | 76.5 | 42.9 | 20.4 | 15.5 | 11.7 | 9.64 | 0.296 | |
| | | SD | 7.04 | 2.56 | 0.434 | 2.95 | 0.784 | 0.232 | 0.492 | 0.363 | | |
| CYP2C19 | Ticlopidine | — | 0 | 0.0412 | 0.123 | 0.370 | 1.11 | 3.33 | 10.6 | 30.0 | — | — |
| | | | μM | μM | μM | μM | μM | μM | μM | μM | | |
| | −NADPH | Average | 100 | 99.6 | 93.4 | 71.2 | 58.5 | 33.8 | 12.8 | 7.72 | 1.40 | 3.10 |
| | | SD | ND | 2.27 | 5.87 | 5.50 | 1.39 | 2.81 | 0.469 | 0.777 | | |
| | +NADPH | Average | 100 | 97.6 | 86.3 | 52.9 | 29.6 | 11.5 | 5.52 | 3.93 | 0.452 | |
| | | SD | ND | 6.02 | 5.78 | 5.63 | 1.11 | 0.922 | 2.57 | 1.15 | | |

^{a}Percent of control enzyme activity = 100 × (Enzyme activity in the presence of inhibitor/Enzyme activity in the absence of inhibitor). Enzyme activity was calculated from the peak area ratio of CYP probe metabolite to IS by LC-MS/MS.
^{b}IC50 shift = IC_{50 (-NADPH)}/IC_{50 (+NADPH)}.
QC for positive control: IC50 shift ≥2.
SD: standard deviation;
ND: not determined (n = 2)

TABLE 27
IC50 Shift for CYP TDI in HLM by Positive Time-dependent Inhibitors (continued)

| | Pre- | | | | | | | | | | | |
|---|---|---|---|---|---|---|---|---|---|---|---|---|
| | incubation | | | | | | | | | | IC₅₀ | IC₅₀ |
| CYP | (30 min) | | % of Control Enzyme Activity (n = 3)^{a} | | | | | | | | (μM) | Shift^{b} |
| CYP2D6 | Paroxetine | — | 0 | 0.0412 | 0.123 | 0.370 | 1.11 | 3.33 | 10.0 | 30.0 | — | — |
| | | | μM | μM | μM | μM | μM | μM | μM | μM | | |
| | −NADPH | Average | 100 | 97.8 | 100 | 92.1 | 78.4 | 54.1 | 33.0 | 19.3 | 4.17 | 19.1 |
| | | SD | 3.39 | 2.05 | 1.37 | 1.51 | 3.88 | 4.23 | 1.96 | 1.39 | | |
| | +NADPH | Average | 100 | 97.9 | 63.9 | 38.0 | 22.0 | 17.2 | 12.8 | 9.62 | 0.218 | |
| | | SD | 4.21 | 6.38 | 2.42 | 1.50 | 0.945 | 0.417 | 0.686 | 0.503 | | |
| CYP3A | Troleandomycin | — | 0 | 0.137 | 0.412 | 1.23 | 3.70 | 11.1 | 33.3 | 100 | — | — |
| (Midazolam) | | | μM | μM | μM | μM | μM | μM | μM | μM | | |
| | −NADPH | Average | 100 | 96.1 | 96.9 | 89.8 | 76.8 | 63.3 | 41.3 | 21.4 | 19.6 | 13.0 |
| | | SD | 5.87 | 2.15 | 1.84 | 1.38 | 0.472 | 0.700 | 3.28 | 1.25 | | |
| | +NADPH | Average | 100 | 75.6 | 66.0 | 50.4 | 36.9 | 26.5 | 18.8 | 10.5 | 1.51 | |
| | | SD | 3.89 | 3.10 | 1.86 | 2.48 | 0.945 | 0.154 | 0.334 | 0.255 | | |
| CYP3A | Troleandomycin | — | 0 | 0.137 | 0.412 | 1.23 | 3.70 | 11.1 | 33.3 | 100 | — | — |
| (Testosterone) | | | μM | μM | μM | μM | μM | μM | μM | μM | | |
| | −NADPH | Average | 100 | 100 | 103 | 91.8 | 85.6 | 75.2 | 55.5 | 39.1 | 44.1 | 49.3 |
| | | SD | 7.63 | ND | 4.64 | 10.6 | 3.22 | 3.31 | 2.19 | 2.58 | | |
| | +NADPH | Average | 100 | 72.4 | 54.8 | 44.9 | 34.4 | 27.8 | 19.5 | 14.4 | 0.894 | |
| | | SD | 6.98 | 3.04 | 1.59 | 0.936 | 0.540 | 0.832 | 0.648 | 0.711 | | |

### Example 6—Transporter Drug-Drug Interaction Assessment

The objective of the current study was to evaluate the substrate and inhibitor potential (IC50) of AMX0035 for the following transporters: P-glycoprotein (P-gp), breast cancer resistance protein (BCRP), organic anion transporting polypeptides 1B1 and 1B3 (OATP1B1 and OATP1B3), organic anion transporters 1 and 3 (OAT1 and OAT3), organic cation transporters 1 and 2 (OCT1 and OCT2), and multidrug and toxin extrusion proteins 1 and 2K (MATE1 and MATE2K). AMX0035 is a combination of TUDCA and PB. In the current study, both TUDCA and PB were present simultaneously in all dosing solutions.

HEK cell lines transfected with each of the uptake transporters of interest (OATP1B1, OATP1B3, OAT1, OAT3, OCT2, MATE1, or MATE2K) were used to determine the inhibition potential of the test article (IC50 assessment). The estimated IC50 toward OAT1 would be between 0.0741 and 0.222 μM TUDCA, with 6.76 and 20.3 μM PB, respectively. The estimated IC50 toward OCT2 would be greater than 162 μM TUDCA and greater than 14785 μM PB. The estimated IC50 for OAT3 would be between 54.0 and 162 μM TUDCA, with 4928 and 14785 μM PB, respectively. The estimated IC50 toward OATP1B1 would be between 12.9 and 38.7 μM TUDCA, with 1208 and 3625 μM PB, respectively. The estimated IC50 toward OATP1B3 would be between 38.7 and 116 μM TUDCA, with 3625 and 10875 μM PB, respectively. The estimated IC50 toward MATE1 would be between 270 and 810 μM TUDCA, with 24642 and 73927 μM PB, respectively. The estimated IC50 of TUDCA and PB toward MATE2K would be greater than 810 μM and greater than 73927 μM, respectively.

The evaluation of AMX0035 as an inhibitor of P-gp was carried out in MDR1-MDCK and C2BBe1 cells. Transport of the P-gp probe substrate digoxin (10 μM) across cell monolayer was used as an index of P-gp activity. The assay was carried out with a bidirectional approach: apical-to-basolateral (AP-to-BL) and basolateral-to-apical (BL-to-AP). In MDR1-MDCK cells, the estimated IC50 toward P-gp would be between 889 and 2668 μM TUDCA, with 7161 and 21482 μM PB, respectively. In C2BBe1 cells, the estimated IC50 toward P-gp would be between 222 and 667 μM TUDCA, with 1790 and 5371 μM PB, respectively.

Evaluation of AMX0035 as an inhibitor of BCRP was carried out in BCRP-MDCK and C2BBe1 cells. Transport of the BCRP probe substrate cladribine (10 μM) across cell monolayer was used as an index of BCRP activity. The assay was carried out with a bidirectional approach. In BCRP-MDCK cells, the estimated IC50 toward BCRP would be between 296 and 889 μM TUDCA, with 2387 and 7161 μM PB, respectively. In C2BBe1 cells, the estimated IC50 toward P-gp would be between 222 and 667 μM TUDCA, with 1790 and 5371 μM PB, respectively.

Evaluation of AMX0035 as a substrate of P-gp and BCRP was conducted in C2BBe1 cells with a bidirectional approach. The efflux ratio of TUDCA was 0.506, 0.305, 0.230, and 0.302 at 81.0, 16.2, 1.62, and 0.810 μM, respectively. The efflux ratio of PB was 0.740, 0.620, 0.940, and 1.65 at 1479, 148, 73.9, and 14.8 μM, respectively.

In summary, in vitro, neither TUDCA nor PB is a substrate of P-gp or BCRP. The estimated IC50 of TUDCA toward each transporter is as follows: 0.0741˜0.222 μM (OAT1), greater than 162 μM (OCT2), 54.0˜162 μM (OAT3), 12.9˜38.7 μM (OATP1B1), 38.7˜116 μM (OATP1B3), 270˜810 μM (MATE1), greater than 810 μM (MATE2K), 889˜2668 μM (P-gp in MDR1-MDCK cells), 222˜667 μM (P-gp in C2BBe1 cells), 296˜889 μM (BCRP in BCRPMDCK cells), and 222˜667 μM (BCRP in C2BBe1 cells). The estimated IC50 of PB toward each transporter is as follows: 6.76˜20.3 μM (OAT1), greater than 14785 μM (OCT2), 4928-14785 μM (OAT3), 1208˜3625 μM (OATP1B1), 3625˜10875 μM (OATP1B3), 24642˜73927 μM (MATE1), greater than 73927 μM (MATE2K), 7161-21482 μM (P-gp in MDR1-MDCK cells), 1790˜5371 μM (P-gp in C2BBe1 cells), 2387˜7161 μM (BCRP in BCRPMDCK cells), and 1790˜5371 μM (BCRP in C2BBe1 cells).

### Introduction

The relevant concentrations of TUDCA and PB are listed in Table 28. The selected test concentrations of TUDCA and PB, based on analytical sensitivity in the current example and clinical relevance, are summarized in Table 29.

For the substrate and inhibitor potential assessment of efflux transporters such as P-gp and BCRP, the preferred in vitro method was cell-based bidirectional permeability assays. In the current example, both human P-gp and BCRP-transfected MDCK and C2BBe1 cells were used. For the substrate and inhibitor potential assessment of uptake transporters such as OATP1B1 and OATP1B3, cell lines with overexpression of the target gene were used.

TABLE 28
Relevant Concentrations of TUDCA and PB
for In Vitro Transporter Assessment

Test Article
AMX0035

| Component | TUDCA | PB |
|---|---|---|
| Molecular Weight (free base) | 499.7 | 163.2 |
| Highest Oral Dose (mg) | 1000 | 3000 |
| [I]₂, μM | 8005 | 64447 |
| Plasma Cₘₐₓ (total [I]₁), μM | 108 | 826 |
| Human Plasma Protein Binding (%)^{a} | 98.5 | 82.1 |
| Unbound Cₘₐₓ (Unbound [I]₁), μM | 1.62 | 148 |
| Unbound [I]_{in, max}, μM^{b} | 3.48 | 326 |

TABLE 29
Concentrations of TUDCA and PB Used in the Current Example

| | Tested Concentrations of | |
|---|---|---|
| | AMX0035 | |
| Assessment | TUDCA (μM) | PB (μM) |
| P-gp and BCRP Substrate | 0.810~81.0 | 14.8~1479 |
| P-gp and BCRP IC₅₀ assessment | 3.66~8005^{a} | 29.5~64447^{a} |
| | 0.915~2001^{b} | 7.37~16112^{b} |
| OATP1B1 and OATP1B3 IC₅₀ | 0.159~348 | 14.9~32625 |
| assessment | | |
| OAT1 IC₅₀ assessment | 0.0741~162 | 6.76~14785 |
| OAT3 IC₅₀ assessment | 0.0741~162 | 6.76~14785 |
| OCT2 IC₅₀ assessment | 0.0741~162 | 6.76~14785 |
| MATE1 and MATE2K IC₅₀ | 0.370~810 | 33.8~73927 |
| assessment | | |

^{a}Used in MDR1-MDCK and BCRP-MDCK cells.
^{b}Used in C2BBe1 cells.

### Methods

### Chemical Stability Assessment

The chemical stability of TUDCA and PB was tested in HBSSg_7.4 and HBSS_8.5. The test article was prepared in DMSO stock (0.5 mM), and aliquots of the DMSO stock were added to each matrix. The nominal concentration of each component was 0.5 μM in each matrix. Aliquots were transferred into separate vials (one vial per time point), and the vials were incubated in a humidified incubator at 37° C. with 5% CO₂. At pre-determined time points, an equal volume of acetonitrile with internal standard was added to each vial, and the mixture was placed at 4° C. until the end of the assay. The peak areas of the test article and IS of each sample were determined by LC-MS/MS, and the ratios of the peak areas (analyte: IS) were calculated.

### Solubility Assessment

All solubility samples were prepared in silanized glass tubes. Aliquots of the powder of each component were added to each matrix in amber vials (Table 30). Following the addition of buffer, samples were incubated in a 37° C. shaking water bath overnight (1020-1080 minutes). After incubation, the mixture was centrifuged at 20800 g for 15 minutes. The supernatant was diluted appropriately, and the concentrations of each component were determined by LC-MS/MS.

TABLE 30
Conditions for Solubility Assessment of TUDCA and PB

| Component | HBSSg_7.4 | HBSSg_8.5 |
|---|---|---|
| TUDCA (nominal conc., μM) | 8005 | 810 |
| PB (nominal conc., μM) | 64447 | 73927 |

### Non-Specific Binding Assessment

Since the assay plates used for the efflux and uptake transporters were made of different materials, the non-specific binding assessment of TUDCA and PB was conducted with the corresponding cell-free plates.

(i) Efflux Transporter Format

Rat-tail collagen-coated Transwell plates (12-well) were used, and all incubations occurred in a humidified incubator at 37° C. with 5% CO2. Aliquots of TUDCA and PB DMSO stock were added to HBSSg_7.4. Triplicate wells were used for the assay (Table 31). The samples were withdrawn at pre-selected time points from the receiver and donor compartments and mixed with an equal volume of acetonitrile containing IS. The concentrations of TUDCA and PB were determined by LC-MS/MS.

(ii) Uptake Transporter Format

Poly-D-lysine-coated plates (24-well) were used, and all incubations occurred in a humidified incubator at 37° C. with 5% CO2. TUDCA and PB dosing solution was prepared in HBSSg_7.4 and HBSSg_8.5. Two treatments (three wells each) were included: in the first treatment, the wells were pre-incubated for 5 minutes with HBSSg_7.4_BSA or HBSSg_8.5_BSA; in the second treatment, no pre-incubation was performed (Table 32). After the pre-incubation, the solution was aspirated, and dosing solution (500 μL) was added to each well. The plate was placed in a humidified incubator at 37° C. with 5% CO2 for 5 minutes. After the incubation, aliquots (200 μL) were withdrawn from each chamber, combined with an equal volume of acetonitrile containing IS, and stored at 4° C. until analysis. The concentrations of TUDCA and PB were determined by LC-MS/MS.

TABLE 31
Non-specific Binding Assessment Conditions (Efflux Transporter Format)

| Matrix Composition | | Sampling (μL) | | Sampling Time (minutes) | |
|---|---|---|---|---|---|
| Donor | Receiver | Donor | Receiver | Donor | Receiver |
| 0.810 μM TUDCA and | HBSSg_7.4 | 50 | 200 | 5 and 120 | 120 |
| 14.8 μM PB in | | | | | |
| HBSSg_7.4 | | | | | |

TABLE 32
Non-specific Binding Assessment Conditions (Uptake Transporter Format)

| Pre-incubation | | Incubation | |
|---|---|---|---|
| Treatment | Time (minutes) | Treatment | Time (minutes) |
| None | None | 0.810 μM TUDCA and 14.8 μM | 5 |
| | | PB in HBSSg_7.4 | |
| HBSSg_7.4_BSA | 5 | | |
| None | None | 0.810 μM TUDCA and 14.8 μM | 5 |
| | | PB in HBSSg_8.5 | |
| HBSSg_8.5_BSA | 5 | | |

### Uptake Format Transporter-Related HEK Cell Culture

Human embryonic kidney (HEK293) cells, transfected with an individual uptake transporter gene (OATP1B1, OATP1B3, OAT1, OAT3, OCT2, MATE1, or MATE2K) or the blank vector control gene (VC), were used to assess the substrate and inhibition potential of the test article toward the corresponding transporter. The cells were maintained in DMEM supplemented with PEST, 10% FBS, 1% NEAA, and 1 mM sodium pyruvate in a humidified incubator at 37° C. with 5% CO2. The culture medium was changed three times weekly, and cell growth was observed by light microscopy. When the cells became confluent, they were harvested by trypsinization and the collected cells were seeded onto plates for the uptake studies. The plates were placed in a humidified incubator at 37° C. with 5% CO2. One day after seeding, the MATE- and vector control-transfected cells were incubated with culture medium containing 10 mM sodium butyrate for approximately 16-20 hours before use.

Prior to the assay (same day), each cell line was checked with a transporter-specific fluorescent marker compound (quality control [QC] compound) to confirm the functionality of the transfected transporter (Table 33).

TABLE 33
Uptake Format Transporter-Transfected HEK Cell Culture Conditions

| | | | | Seeding | | |
|---|---|---|---|---|---|---|
| | QC | | Incubation | Density | Plate | Selection |
| Cell Line | Compound | | (minutes) | (per cm²) | Type | Reagent |
| OATP1B1-, OATP1B3-, | 1 | μM FMTX | 10 | 1.25 × 10⁵ | 24- | 800 μg/mL |
| and VC-Cells | | | | | coated | G418 |
| OAT1- and VC-Cells | 5 | μM 6-CF | 5 | | well, | |
| OAT3- and VC-Cells | 20 | μM 5-CF | 10 | | poly- | |
| OCT2- and VC-Cells | 1 | μM ASP | 5 | | D-lysine- | |
| MATE1-, MATE2K-, | 1 | μM ASP^{a} | 5 | | coated | 5 μg/mL |
| and VC-Cells | | | | | | puromycin |

^{a}ASP was prepared in HBSSg_8.5; cells were pre-incubated with HBSSg_8.5 for 20 minutes prior to the QC assay.

### Certification of HEK Cells for the Uptake Format Transporter Study

The QC dosing solutions were prepared by diluting an aliquot of DMSO stock of QC compound in the appropriate buffer based on the transporter; the final concentration of each QC compound is shown in Table 33. The medium was gently aspirated without disturbing the cells. The QC dosing solution was added to each well, and the cells were incubated for a pre-selected period in a humidified incubator at 37° C. with 5% CO2. The reaction was stopped by washing the cells twice with ice-cold HBSSg_7.4 buffer. After the second rinse, the cells were lysed with 150 L RIPA buffer, and incubated for 10 minutes at 4° C. An aliquot of the supernatant (100 μL) was collected into an opaque, white fluorescence reader plate, and the fluorescence of each compound was read with optimal excitation and emission wavelengths.

Additional wells (n=3) from the same batch of cells were used for protein determination. The medium was gently aspirated without disturbing the cells, and the wells were washed twice with HBSSg buffer. After the second rinse, the cells were lysed with 150 μL RIPA buffer on a shaker at 4° C. for 15 minutes. Aliquots of the cell lysates (12.5 μL) were collected in microcentrifuge tubes and stored at −20° C. until protein concentration analysis.

### Format Transporter-Related Cell Culture

C2BBe1 and MDCK cells were obtained from American Type Culture Collection (Manassas, Va., USA). MDR1-MDCK cells were obtained from National Institute of Health (Bethesda, Md., USA). BCRP-MDCK cells were generated by Absorption Systems LLC (Exton, Pa., USA). The cells were maintained in DMEM medium containing 10% FBS, 1% NEAA, 4 mM L-glutamine, 1 mM sodium pyruvate, PEST, and appropriate selecting reagents in a humidified incubator at 37° C. with 5% C02 (Table 34). The culture medium was changed three times weekly, and cell growth was observed by light microscopy. When the cells became 80-90% confluent, they were harvested by trypsinization and seeded onto Costar Transwell plates (60,000 cells/cm²) to grow cell monolayers for the permeability studies. Fresh medium (1.5 mL) was added to each well, and cell suspension (0.5 mL) was added to each insert. The plates were placed in a humidified incubator at 37° C. with 5% CO2, and the culture medium was changed every other day until use. Prior to the use of BCRP-MDCK cells, the cell monolayers were supplemented with medium containing sodium butyrate (2.5 mM) overnight (approximately 16-20 hours).

TABLE 34
Efflux Format Transporter-Related Cell Culture Conditions

| Cell Line | Selected Reagent | Plate Type | Seeding Density |
|---|---|---|---|
| C2BBe1 | None | 12-Transwell | 60.000 cells/cm² |
| MDCK | None | coated with rat- | |
| MDR1-MDCK | 80 ng/mL colchicine | tail collagen | |
| BCRP-MDCK | 800 μg/mL G418 | | |

### Certification of Cells for the Efflux Transporter-Related Assays

Prior to the assay, the permeability of QC compounds was measured in each batch of cell monolayers. The QC compounds were prepared in HBSSg_7.4. The cell monolayers used for the QC tests were washed twice with HBSSg_7.4. For apical-to-basolateral (AP-to-BL) transport, 0.5 mL of dosing solution with QC compounds was added to the inserts and 1.5 mL of HBSSg_7.4 was added to the well. For basolateral-to-apical (BL-to-AP) transport, 1.5 mL of dosing solution was added to the well, and 0.5 mL of HBSSg_7.4 was added to the insert. The cells were incubated in a humidified incubator for 120 minutes. Samples were taken from the receiver compartment at 120 minutes, and the concentrations of each QC compound were determined by LC-MS/MS.

### Tolerability Assessment in Efflux Transporter-Related Cells

The effect of TUDCA and PB on cell monolayer integrity was assessed in C2BBe1 and MDCK cells (Table 35). MDCK was used as a representative cell line of MDR1-MDCK and BCRPMDCK cells. Triplicate wells of cell monolayers of each cell line were used for each treatment. The dosing solutions were prepared in HBSSg_7.4 containing LY. Dosing solution and plain HBSSg_7.4 were placed on the BL and AP sides, respectively, for 150 minutes in a humidified incubator at 37° C. with 5% CO2. After incubation, aliquots from the AP side (100 μL) were collected, and the concentrations of LY were determined by fluorescence using a BMG microplate reader with excitation and emission wavelengths at 485 nm and 540 nm, respectively. The concentrations of TUDCA and PB in the dosing solutions were determined by LC-MS/MS.

TABLE 35
Tolerability Assessment Conditions (Efflux Transporter)

| | | | Sampling | |
|---|---|---|---|---|
| Matrix Composition | | Incubation | (μL) | |
| AP | BL | (minutes) | AP | BL |
| HBSSg_7.4 Only | TUDCA and PBª in HBSSg_7.4 with LY^{c} | 150 | 100 | None |
| HBSSg_7.4 Only | HBSSg_7.4 with LY^{c} | | | |

^{a}The concentrations of TUDCA and PB, respectively, were 8005 and 64447 μM, 4002 and 32223 μM, 3202 and 25778 μM, 2001 and 16112 μM, and 800 and 6445 μM.
^{b} The concentration of LY was 200 μM.

### Tolerability Assessment in Uptake Format Transporter-Transfected Cells

TUDCA and PB were prepared in the appropriate assay buffers at pre-determined concentrations (Table 36). The culture medium was aspirated. Vector control-transfected cells (group 1) received a 30-minute pre-incubation with test article dosing solution, whereas vector control-transfected cells (group 2) did not receive the pre-incubation. For MATEs-related tolerability assays, the cells were pre-incubated with plain HBSSg_8.5 for 20 minutes. After pre-incubation, the pre-incubation solution was aspirated and the appropriate buffer or test article-containing dosing solution (500 μL) was added to the corresponding wells. The cells were placed in a humidified incubator at 37° C. with 5% CO2. After 20 minutes, the buffer or dosing solution was removed from the wells, the cells were rinsed twice with HBSSg_7.4, and 500 μL of culture medium was added. Subsequently, 100 μL of the CellTiter 96® AQueous ONE Solution Cell Proliferation Assay reagent was added to each well, and the cells were then returned to a humidified incubator at 37° C. with 5% CO2. After one hour of incubation, absorbance of each well was measured at 492 nm using a FLUOstar® spectrophotometer (BMG Labtech, Ortenberg, Germany). The concentrations of TUDCA and PB in the dosing solutions were determined by LC-MS/MS. The vector control-transfected HEK cells were used as the representative cell line in the tolerability assessment. Puromycin-resistant vector control cells (Puro-VC) were for MATE-related cells, and G418-resistant vector control (Neo-VC) were for all other transporter-transfected cells.

The CellTiter 96 AQueous Non-Radioactive Cell Proliferation Assay is a homogeneous, colorimetric method for determining the number of viable cells in cytotoxicity assays. The CellTiter 96 AQueous Assay is composed of solutions of a tetrazolium compound (MTS; 3-(4, 5-dimethylthiazol-2-yl)-5-(3-carboxymethoxyphenyl)-2-(4-sulfophenyl)-2H-tetrazolium, inner salt) and an electron coupling reagent (phenazine methosulfate). MTS is bioreduced by cells into a formazan product that is soluble in tissue culture medium. The quantity of formazan product, as measured by the amount of absorbance at 492 nm, is directly proportional to the number of living cells in culture.

### Uptake Format Transporter Inhibitor Assessment

TUDCA and PB were prepared in the appropriate matrices at pre-selected concentrations (Table 37). The culture medium was gently aspirated; and, the inhibition assay was initiated as follows: 1) incubation at 37° C. with 5% CO2 with 500 μL of probe substrate in the absence and presence of TUDCA and PB or known inhibitor for the desired time; 2) at the end of the incubation period, the solution was gently aspirated; the cells were rinsed twice with ice-cold HBSSg_7.4 buffer (1000 μL per rinse); 3) the cells were lysed in 400 μL acetonitrile:water (3:1, v/v) containing IS; and 4) the lysates were collected for analysis of the probe substrate concentration (Table 38). The dosing solution concentrations of TUDCA and PB in the inhibitor assessment were also determined by LC-MS/MS.

TABLE 36
Tolerability Assay Conditions (Uptake Format Transporters)

| | | TUDCA and PB | | Incubation |
|---|---|---|---|---|
| Treatment | Cell Lines | Conc. (μM) | Replicates | (minutes) |
| TUDCA and PB | Neo-VC-transfected | (174 and 16312) μM^{a,b} | 3 | 20 |
| groups (group 1) | Cells | (348 and 32625) μM^{a,b} | | |
| Buffer only group | Neo-VC-transfected | NA^{c} | 3 | |
| (Control 1) | Cells | | | |
| TUDCA and PB | Neo-VC-transfected | (174 and 16312) μM^{a} | 3 | |
| groups (group 2) | Cells | (348 and 32625) μM^{a} | | |
| Buffer only group | Neo-VC-transfected | NA | 3 | |
| (Control 2) | Cells | | | |
| TUDCA and PB | Puro-VC-transfected | (405 and 36964) μM^{e} | 3 | 20 |
| groups | Cells^{d} | (810 and 73927) μM^{e} | | |
| Buffer only group | Puro-VC-transfected | NA | 3 | |
| (Control) | Cells^{d} | | | |

TABLE 37
Concentrations of TUDCA and PB in the
Uptake Transporter IC50 Assessment

| | TUDCA | PB |
|---|---|---|
| Annotation of Solution | Nominal Conc. (μM) | Nominal Conc. (μM) |
| Solution 1^{a,b} | 162 | 14785 |
| Solution 2^{a,b} | 54.0 | 4928 |
| Solution 3^{a,b} | 18.0 | 1643 |
| Solution 4^{a,b} | 6.00 | 548 |
| Solution 5^{a,b} | 2.00 | 183 |
| Solution 6^{a,b} | 0.667 | 60.8 |
| Solution 7^{a,b} | 0.222 | 20.3 |
| Solution 8^{a,b} | 0.0741 | 6.76 |
| Solution 1^{a,c} | 348 | 32625 |
| Solution 2^{a,c} | 116 | 10875 |
| Solution 3^{a,c} | 38.7 | 3625 |
| Solution 4^{a,c} | 12.9 | 1208 |
| Solution 5^{a,c} | 4.30 | 403 |
| Solution 6^{a,c} | 1.43 | 134 |
| Solution 7^{a,c} | 0.48 | 44.8 |
| Solution 8^{a,c} | 0.159 | 14.9 |
| Solution 1^{d} | 810 | 73927 |
| Solution 2^{d} | 270 | 24642 |
| Solution 3^{d} | 90.0 | 8214 |
| Solution 4^{d} | 30.0 | 2738 |
| Solution 5^{d} | 10.0 | 913 |
| Solution 6^{d} | 3.33 | 304 |
| Solution 7^{d} | 1.11 | 101 |
| Solution 8^{d} | 0.370 | 33.8 |

^{a}The dosing solution was prepared in HBSSg_7.4.
^{b}The dosing solutions were used for OAT1, OAT3, and OCT2 IC50 assessment.
^{c}The dosing solutions were used for OATP1B1 and OATP1B3 IC50 assessment.
^{d}The dosing solution was prepared in HBSSg_8.5 and used for MATE1 and MATE2K IC50 assessment.

TABLE 38
Uptake Format Transporter Inhibitor (IC50) Assessment Conditions

| | Probe | | Test Article And Known | Lysis |
|---|---|---|---|---|
| | Substrate | Incubation | Inhibitor^{b} | Solution |
| Cell Lineª | (Concentration) | (minutes) | (Concentration) | (μL) |
| OATP1B1-, | Atorvastatin | 5 | No test article or inhibitor | 400 |
| OATP1B3-, and Vector | (0.15 μM)^{d} | | TUDCA and PB (listed in | |
| Control-transfected | | | Table 12) | |
| Cells^{c} | | | Rifamycin SV (10 μM) | |
| OAT1- and Vector | PAH (10 μM)^{d} | 5 | No test article or inhibitor | |
| Control-transfected | | | TUDCA and PB (listed in | |
| Cells | | | Table 12) | |
| | | | Probenecid (100 μM) | |
| OAT3- and Vector | Furosemide | 5 | No test article of inhibitor | |
| Control-transfected | (5 μM)^{d} | | TUDCA and PB (listed in | |
| Cells | | | Table 12) | |
| | | | Probenecid (100 μM) | |
| OCT2- and Vector | MPP⁺ (5 μM)^{d} | 2 | No test article or inhibitor | |
| Control-transfected | | | TUDCA and PB (listed in | |
| Cells | | | Table 12) | |
| | | | Imipramine (300 μM) | |
| MATE1- and Vector | Metformin | 10 | No test article or inhibitor | |
| Control-transfected | (50 μM)^{f} | | TUDCA and PB (listed in | |
| Cells^{e} | | | Table 12) | |
| | | | Cimetidine (100 μM) | |
| MATE2K-, and Vector | Metformin | 10 | No test article or inhibitor | |
| Control-transfected | (50 μM)^{f} | | TUDCA and PB (listed in | |
| Cells^{e} | | | Table 12) | |
| | | | Pyrimethamine (1 μM) | |

^{a}Duplicate wells per cell line were used per treatment.
^{b}Unless specified otherwise, no pre-incubation was performed.
^{c}The cells were pre-incubated with test article, RSV, or plain HBSSg_7.4 for 30 minutes prior to the assay.
^{d}The dosing solutions were prepared with HBSSg_7.4.
^{e}The cells were pre-incubated with HBSSg_8.5 for 20 minutes prior to the inhibition assessment.
^{f}The dosing solutions were prepared with HBSSg_8.5.

### P-gp and BCRP IC50 Assessment

The assessment was conducted in both MIDR1-MDCK and C2BBe1 cells (for P-gp) and BCRPMDCK and C2BBe1 cells (for BCRP). TUDCA and PB were prepared in the appropriate matrices at pre-selected concentrations (Table 39). The bidirectional transport of digoxin was measured in the absence and presence of TUDCA and PB, valspodar (with digoxin only), and Ko143 (with cladribine only). Digoxin and cladribine were used as probe substrates for P-gp and BCRP, respectively (Table 40). All incubations were in a humidified incubator at 37° C. with 500 CO₂. The probe substrates, known inhibitors, and TUDCA and PB were prepared in HIBSSg_7.4. All probe substrate-containing solutions also contained 200 μM LY.

A 30-minute pre-incubation with TUDCA and PB or known inhibitor was performed on both sides to pre-load the cells. After 30 minutes, the pre-incubation solution was aspirated. Aliquots of fresh dosing solution with or without TUDCA and PB or known inhibitor (0.55 mL for AP-to-BL, 1.55 mL for BL-to-AP) were added to the donor compartment and HBSSg_7.4 buffer with or without TUDCA and PB or known inhibitor (1.5 mL for AP-to-BL, 0.5 mL for BL-to-AP) were added to the receiver compartment. Receiver samples (300 μL) were taken at pre-selected time points. For the receiver samples, one part (200 μL) was used for the analysis of TUDCA and PB, and the rest of the sample (100 μL) was collected for LY measurement at the end of permeability assay. Donor samples (50 μL) were taken at pre-selected time points.

After the completion of the permeability assay, cell monolayer integrity was examined with LY assessment. The concentration of LY was measured with 438 nm (excitation) and 540 nm (emission). The concentrations of the probe substrate in the collected dosing solution, donor, and receiver samples, as well as TUDCA and PB in dosing solutions, were determined by LC-MS/MS.

TABLE 39
Concentrations of TUDCA and PB in
the P-gp and BCRP IC50 Assessment

| | TUDCA | PB |
|---|---|---|
| Annotation of Solution | Nominal Conc. (μM) | Nominal Conc. (μM) |
| Solution 1^{a,b} | 8005 | 64447 |
| Solution 2^{a,b} | 2668 | 21482 |
| Solution 3^{a,b} | 889 | 7161 |
| Solution 4^{a,b} | 296 | 2387 |
| Solution 5^{a,b} | 98.8 | 796 |
| Solution 6^{a,b} | 32.9 | 265 |
| Solution 7^{a,b} | 11.0 | 88.4 |
| Solution 8^{a,b} | 3.66 | 29.5 |
| Solution 1^{a,c} | 2001 | 16112 |
| Solution 2^{a,c} | 667 | 5371 |
| Solution 3^{a,c} | 222 | 1790 |
| Solution 4^{a,c} | 74.1 | 597 |
| Solution 5^{a,c} | 24.7 | 199 |
| Solution 6^{a,c} | 8.23 | 66.3 |
| Solution 7^{a,c} | 2.74 | 22.1 |
| Solution 8^{a,c} | 0.915 | 7.37 |

^{a}The dosing solution was prepared in HBSSg_7.4.
^{b}The dosing solutions were used in MDR1-MDCK and BCRP-MDCK cells.
^{c}The dosing solutions were used in C2BBe1 cells.

TABLE 40
P-gp and BCRP IC50 Assessment Conditions

| | | | Sampling | | Sampling | |
|---|---|---|---|---|---|---|
| | Matrix Composition | | (μL) | | (minutes) | |
| Pre-incubation | AP | BL | AP | BL | AP | BL |
| HBSSg_7.4 Alone, | Probe Substrate^{b} in | HBSSg_7.4 in the | 50 | 300 | 5, 120 | 120 |
| TUDCA and PB^{a} | the absence or | absence or presence | | | | |
| or Known | presence of | of TUDCA and PBª | | | | |
| Inhibitor^{c} | TUDCA and PBª or | or known inhibitor^{c} | | | | |
| | known inhibitor^{c} | | | | | |
| HBSSg_7.4 Alone, | HBSSg_7.4 in the | Probe Substrate^{b} in the | 300 | 50 | 120 | 5, 120 |
| TUDCA and PB^{a} | absence or presence | absence or presence of | | | | |
| or Known | of TUDCA and PB^{a} | TUDCA and PBª or | | | | |
| Inhibitor^{c} | or known inhibitor^{c} | known inhibitor^{c} | | | | |

^{a}The concentrations of TUDCA and PB were listed in Table 39.
^{b}The concentration of the probe substrate was 10 μM for digoxin, and 10 μM for cladribine.
^{c}Valspodar (1 μM) was used for P-gp inhibition; Ko143 (0.5 μM) was used for BCRP inhibition.

### P-gp and BCRP Substrate Assessment

The P-gp and BCRP substrate assessment was conducted in C2BBe1 cell monolayers. TUDCA and PB were prepared in the appropriate matrices at pre-selected concentrations (Table 41). All incubations were in a humidified incubator at 37° C. with 5% CO2. LY was co-dosed with each dosing solution at a nominal concentration of 200 μM. The bidirectional permeability assay was run with TUDCA and PB in the absence of P-gp or BCRP inhibitor (Tables 42). Digoxin (P-gp substrate) or cladribine (BCRP substrate) was run in parallel (Table 43).

The cell culture medium was aspirated, and the cell monolayers were rinsed once with HBSSg_7.4. For the TUDCA and PB group, aliquots of fresh dosing solution (0.55 mL for APto-BL, 1.55 mL for BL-to-AP) were added to the donor compartment, and HBSSg_7.4 buffer without inhibitor (1.5 mL for AP-to-BL, 0.5 mL for BL-to-AP) were added to the receiver compartment. For receiver samples, aliquots (300 μL) were taken at pre-selected time points. For the receiver samples, one part (200 μL) was used for the analysis of TUDCA and PB, and the rest of the sample (100 μL) was collected for LY measurement at the end of the permeability assay. For donor samples, aliquots (50 μL) were taken at pre-selected time points without replacement.

The concentrations of TUDCA and PB, digoxin, and cladribine in dosing solution, receiver, and donor samples were determined by LC-MS/MS. The concentrations of LY were determined by fluorescence using a BMG microplate reader with excitation and emission wavelengths at 485 nm and 540 nm, respectively.

TABLE 41
Concentrations of TUDCA and PB in the
P-gp and BCRP Substrate Assessment

| | TUDCA | PB |
|---|---|---|
| Annotation of Solution | Nominal Conc. (μM) | Nominal Conc. (μM) |
| Solution 1ª | 81.0 | 1479 |
| Solution 2ª | 16.2 | 148 |
| Solution 3ª | 1.62 | 73.9 |
| Solution 4ª | 0.810 | 14.8 |

ªThe dosing solution was prepared in HBSSg_7.4.

TABLE 42
Efflux Transporter Substrate Assessment Condition 1

| | | | Sampling | | Sampling | |
|---|---|---|---|---|---|---|
| | | | Volume | | Time Points | |
| | Matrix Composition | | (μL) | | (minutes) | |
| Treatments | AP | BL | AP | BL | AP | BL |
| | AP-to-BL | | | | | |
| TUDCA | HBSSg_7.4 | HBSSg_7.4_ | 50 | 300 | 5, 120 | 60, 120 |
| and PB^{a} | | BSA | | | | |
| | BL-to-AP | | | | | |
| TUDCA | HBSSg_7.4_ | HBSSg_7.4 | 300 | 50 | 60, 120 | 5, 120 |
| and PBª | BSA | | | | | |

TABLE 43
Efflux Transporter Substrate Assessment Condition 2

| | | Sampling | | | |
|---|---|---|---|---|---|
| Matrix Composition | | Volumes | | Sampling Time | |
| | Basolateral | (μL) | | Points (minutes) | |
| Apical (AP) | (BL) | AP | BL | AP | BL |
| Probe Substrate | HBSSg_7.4 | 50 | 300 | 5, 120 | 120 |
| with LY in | | | | | |
| HBSSg_7.4^{a} | | | | | |
| HBSSg_7.4 | Probe Substrate | 300 | 50 | 120 | 5, 120 |
| | with LY in | | | | |
| | HBSSg_7.4^{a} | | | | |

^{a}The concentration of digoxin and cladribine was 10 μM each.

### Data Analysis

### Uptake Transporter-Related Analysis

The influx rates of TUDCA and PB in the transporter—and vector control—transfected cells were determined. Negative values of percent inhibition are reported as 0, and the maximal inhibition is reported as 100%. The IC50 values were calculated with GraphPad Prism (version 5.0).

$$Influx rate ( I R ) = \frac{( \frac{{C}_{S} \times {V}_{S}}{{C}_{P} \times {V}_{P}} )}{Assay duration} \qquad ( 1 )$$

$$Influx rate ratio = \frac{I {R}_{( TF )}}{I {R}_{( VC )}} \qquad ( 2 )$$

$$Net influx rate ( N I R ) = I {R}_{( TF )} - I {R}_{( VC )} \qquad ( 3 )$$

$$Percent inhibition = ( 1 - ( \frac{{( I {R}_{TF} )}_{TA} - {( I {R}_{VC} )}_{TA}}{{( I {R}_{TF} )}_{No TA} - {( I {R}_{VC} )}_{No TA}} ) ) \times 100 \qquad ( 4 )$$

$$Percent remaining = ( \frac{{( I {R}_{TF} )}_{TA} - {( I {R}_{VC} )}_{TA}}{{( I {R}_{TF} )}_{No TA} - {( I {R}_{VC} )}_{No TA}} ) \times 100 \qquad ( 5 )$$

$$Y = {Y}_{L} + \frac{{Y}_{H} {Y}_{L}}{1 + {10}^{( {logIC}_{50} - X ) \times Hill slope}} \qquad ( 6 )$$

IR: average influx rate (amount of substrate normalized to average protein content and incubation time, pmol/mg protein/minute)
CS: the substrate concentration in the cell lysate in M
VS: the substrate assessment cell lysate volume in mL
CP: the protein concentration in the cell lysate in mg/mL
VP: the protein content determination cell lysate volume in mL
Vesicle protein: protein content of the vesicles (50 kg)
VC: vector control-transfected cells
TF: transporter-transfected cells
TA: TUDCA and PB or known inhibitor
ATP: in the presence of ATP
AMP: in the presence of AMP
X: logarithm of the nominal concentration of TUDCA and PB
Y: percent activity remaining of transporter at a given concentration
YL: lowest response (with the highest concentration of TUDCA and PB)
YH: highest response (vehicle control)

### Efflux Transporter-Related Analysis

$${P}_{app} = \frac{{dC}_{R}^{c} \times {V}_{R}}{dt \times A \times {C}_{D 0} \times 60} \qquad ( 7 - A )$$

$${P}_{app} = \frac{{C}_{R}^{60} / 60 \times {V}_{R}}{A \times {C}_{D 0} \times 60} \qquad ( 7 - B )$$

$${P}_{app} = \frac{{C}_{R}^{final} / 120 \times {C}_{R}}{A \times {C}_{D 5} \times 60} \qquad ( 7 - C )$$

$${P}_{app} = \frac{{C}_{R}^{final} / 60 \times {V}_{R}}{A \times {C}_{DN} \times 60} \qquad ( 7 - D )$$

$${P}_{app} = \frac{{C}_{R}^{final} / 120 \times {V}_{R}}{A \times {C}_{DN} \times 60} \qquad ( 7 - E )$$

$$Percent recovery = 100 \times \frac{{C}_{R}^{final} \times {V}_{R} + {C}_{D}^{final} \times {V}_{D}}{{V}_{D} \times {C}_{DN}} \qquad ( 8 )$$

$$Efflux ratio = \frac{{P}_{app} ( BL - to - AP )}{{P}_{app} ( AP - to - BL )} \qquad ( 9 )$$

$$Corrected efflux ratio = Efflux ratio - 1 \qquad ( 10 )$$

$$Percent inhibition = ( 1 - \frac{corrected efflux {ratio}_{TA}}{corrected efflux {ratio}_{no TA}} ) \times 100 \qquad ( 11 )$$

$$Percent inhibition = \frac{corrected efflux {ratio}_{TA}}{corrected efflux {ratio}_{no TA}} \qquad ( 12 )$$

dC_{R}/dt: the slope of the cumulative concentration in the receiver compartment versus time (μM/s);
C_{R}^{C}: cumulative receiver concentration (μM);
C_{R}⁶⁰: receiver concentration at 60 minutes (μM);
V_{R}: volume of the receiver compartment (mL);
V_{D}: volume of the donor compartment (mL);
A: cell membrane surface area (1.13 cm²);
C_{D0}: 0-minute donor concentration (μM);
C_{D5}: 5-minute donor concentration (μM);
C_{DN}. nominal concentration of dosing solution (μM);
C_{R}^{final}: cumulative receiver concentration at the end of the incubation (μM);
C_{D}^{final}: donor concentration at the end of the incubation (μM);
TA: test article or known inhibitor

Because of the withdrawal and replacement in the receiver side, cumulative concentrations were calculated before the slope was constructed. The cumulative concentration at each time point, C_{R}^{C}, was equal to the sum of the measured concentration at that time point (Cₘ) and 3/15 (3/5 for BL-to-AP direction) of the measured concentrations at the previous time points since 300 μL out of the 1.5 mL (or 300 μL out of 0.5 mL for BL-to-AP direction) total sample was withdrawn and replaced.

For permeability tests,

C_{R}^{C}60=Cₘ60

C_{R}^{C}120=Cₘ120+3/15(or 3/5)×Cₘ60

Equations (7-A) and (7-B) were used to determine the Pₐₚₚ of TUDCA and PB. Equation (7-C) was used to determine the Pₐₚₚ of digoxin and cladribine. Equations (7-D) and (7-E) were used to determine the Pₐₚₚ of LY. Equation (7-E) was used to determine the Pₐₚₚ of control compounds in the batch QC of cell monolayers.

Since no 0-minute receiver sample was taken, the origin was not included in the fit. The rationale for equation (10) is as follows: an efflux ratio of 1 indicates that there is zero efflux. Therefore, the value of 1 was subtracted from the calculated efflux ratios before determining the remaining efflux. The minimal value of the percent inhibition was “0%”, and the maximal value “100%”.

### Results

### Chemical Stability

TUDCA and PB appeared to be stable in the tested matrices during the tested period (Table 44). In the subsequent solubility assessment, TUDCA and PB were incubated overnight.

TABLE 44
Chemical Stability of TUDCA and PB

| | | | Sampling Time (minutes) | | |
|---|---|---|---|---|---|
| Component | Matrix | Parameters | 0 | 120 | Overnight^{a} |
| TUDCA | HBSSg_7.4 | Peak Area Ratio | 6.99 | 7.06 | 6.66 |
| | | Percent of the 0- | 100 | 101 | 95.3 |
| | | minute value | | | |
| | HBSSg_8.5 | Peak Area Ratio | 6.65 | 6.04 | 6.42 |
| | | Percent of the 0- | 100 | 90.8 | 96.5 |
| | | minute value | | | |
| PB | HBSSg_7.4 | Peak Area Ratio | 1.37 | 1.36 | 1.44 |
| | | Percent of the 0- | 100 | 99.3 | 105 |
| | | minute value | | | |
| | HBSSg_8.5 | Peak Area Ratio | 1.41 | 1.12 | 1.39 |
| | | Percent of the 0- | 100 | 79.4 | 98.6 |
| | | minute value | | | |

^{a}1020-1080 minutes.

### Solubility Assessment

The acceptance criterion of solubility assessment is that the measured concentration in the matrix must be in the range of 80-120% of the nominal value in at least two of three replicates.

In the solubility assessment, the measured concentrations of TUDCA and PB were greater than 80% of the nominal concentration in at least two replicates in both matrices (Table 45). The results indicate that TUDCA and PB are soluble up to the target concentration: 8005 M in HBSSg_7.4 and 810 μM HBSSg_8.5 for TUDCA, and 64447 μM in HBSSg_7.4 and 73927 μM in HBSSg_8.5 for PB.

TABLE 45
Solubility Measurements of TUDCA and PB

| | | | Measured | Percent of the Nominal |
|---|---|---|---|---|
| Component | Matrix | Replicate | Concentration (μM) | Concentration |
| TUDCA | HBSSg_7.4 | 1 | 6710 | 83.8 |
| | | 2 | 6950 | 86.8 |
| | | 3 | 6770 | 84.6 |
| | HBSSg_8.5 | 1 | 719 | 88.8 |
| | | 2 | 704 | 86.9 |
| | | 3 | 704 | 86.9 |
| PB | HBSSg 7.4 | 1 | 52000 | 80.7 |
| | | 2 | 59300 | 92.0 |
| | | 3 | 52800 | $1.9 |
| | HBSSg_8.5 | 1 | 62200 | 84.1 |
| | | 2 | 53200 | 72.0 |
| | | 3 | 61100 | 82.6 |

### Non-Specific Binding Assessment

The acceptance criterion for dosing solution preparation is that the measured concentration of the dosing solution must be in the range of 80-120% of the nominal value in at least two of three replicates. The measured dosing concentrations of TUDCA and PB in all three replicates were in the range of 80-120% of the nominal value for each treatment, indicating that the preparation of the dosing solution was acceptable.

For the efflux transporter format, the recovery of TUDCA and PB was greater than 80% in all three replicates, indicating that the test article has little non-specific binding to the experimental device. No additional procedures were used in the efflux transporter substrate assessment.

For the uptake transporter format, the recovery of TUDCA and PB was greater than 90% without BSA pre-incubation in both matrices. The results indicate that TUDCA and PB has very little non-specific binding to the experimental device. In the uptake transporter assessments, BSA pre-incubation was not performed.

### Tolerability Assessment in Efflux Transporter-Related Cells

The concentrations of TUDCA and PB in the efflux transporter-related tolerability assessment dosing solutions were analyzed. The measured concentration of the test article in all three replicates was within 80-120% of the nominal value of each concentration, indicating that the preparation of the dosing solutions was acceptable.

In Run 1, the Pₐₚₚ of LY was greater than 0.8×10−6 cm/s in all monolayers of C2BBe1 in the presence of TUDCA and PB (Table 46), indicating that cell monolayer integrity was affected by the presence of the test articles. In Run 2, three lower concentrations of TUDCA and PB were tested, and the Pₐₚₚ of LY was greater than 0.8×10−6 cm/s in monolayers in the presence of TUDCA and PB at 3202 μM and 25778 μM, respectively, but it was less than 0.8×10−6 cm/s at two other concentrations (Table 46).

In the following assays, the highest concentration of TUDCA and PB did not exceed 2001 μM and 16112 μM, respectively, in C2BBe1 cells.

TABLE 46
Effect of TUDCA and PB on Integrity of C2BBel Cell Monolayers

| | | Lucifer Yellow Pₐₚₚ |
|---|---|---|
| Cell Line | Treatment | (10⁻⁶ cm/s)^{c} |
| C2BBe1 | HBSSg_7.4 Controlª | 0.5 |
| | | 0.5 |
| | | 0.5 |
| | 4002 μM TUDCA and 32223 μM PB | >1.2^{d} |
| | with LY in HBSSg_7.4ª | >1.2^{d} |
| | | >1.2^{d} |
| | 8005 μM TUDCA and 64447 μM PB | >1.2^{d} |
| | with LY in HBSSg_7.4^{a} | >1.2^{d} |
| | | >1.2^{d} |
| | HBSSg_7.4 Control^{b} | 0.3 |
| | | 0.3 |
| | | 0.4 |
| | 800 μM TUDCA and 6445 μM PB with | 0.3 |
| | LY in HBSSg_7.4^{b} | 0.2 |
| | | 0.3 |
| | 2001 μM TUDCA and 16112 μM PB | 0.6 |
| | with LY in HBSSg_7.4^{b} | 0.5 |
| | | 0.6 |
| | 3202 μM TUDCA and 25778 μM PB | >1.2^{d} |
| | with LY in HBSSg_7.4^{b} | <1.2^{d} |
| | | >1.2^{d} |

^{a}Run 1.
^{b}Run 2
^{c}Acceptance criterion is LY Pₐₚₚ ≤ 0.8 × 10⁻⁶ cm/s.
^{d}The measured concentration of LY was greater than 5 μM (the upper limit of quantification). The value of 5 was used to estimate the Pₐₚₚ of LY. The actual Pₐₚₚ of LY was greater than 1.2 × 10⁻⁶ cm/s.

For MDCK cells, the Pₐₚₚ of LY was less than 0.8×10−6 cm/s in all monolayers in the presence of TUDCA and PB (Table 47), indicating that the cell monolayers were not affected by the presence of the test articles.

In the following assays, the highest concentration of TUDCA and PB did not exceed 8005 μM and 64447 μM, respectively, in MDR1- or BCRP-transfected MDCK cells.

TABLE 47
Effect of TUDCA and PB on Integrity of MDCK Cell Monolayers

| | | Lucifer Yellow Pₐₚₚ |
|---|---|---|
| Cell Line | Treatment | (10⁻⁶ cm/s)^{a} |
| MDCK | HBSSg_7.4 Control | <0.1^{b} |
| | | <0.1^{b} |
| | | <0.1^{b} |
| | 4002 μM TUDCA and 32223 μM PB | 0.2 |
| | with LY in HBSSg_7.4 | 0.3 |
| | | 0.3 |
| | 8005 μM TUDCA and 64447 μM PB | 0.3 |
| | with LY in HBSSg_7.4 | 0.5 |
| | | 0.3 |

^{a}Acceptance criterion is LY Pₐₚₚ ≤ 0.8 × 10⁻⁵ cm/s.
^{b}The measured concentration of LY was less than 0.313 μM (the lower limit of quantification). The value of 0.313 was used to estimate the Pₐₚₚ of LY. The actual Pₐₚₚ of LY was less than 0.1 × 100⁻⁶ cm/s.

### Tolerability Assessment in Uptake Transporter-Related Cells

The concentrations of TUDCA and PB in the uptake transporter-related tolerability assessments were analyzed. The measured concentrations in at least two of three replicates were within 80-120% of the nominal value in both pH 7.4 and pH 8.5 dosing solutions, indicating that the preparation of the dosing solutions was acceptable.

In the cells that were pre-incubated with TUDCA and PB for 30 minutes, the formazan absorbance was greater than 75% of the control value in all groups (Table 48). The results indicate that TUDCA and PB at the tested concentrations had no adverse effect on the metabolic activity of the cells. In the subsequent assays, the highest concentration of TUDCA and PB did not exceed 348 and 32625 μM, respectively, in these cells.

In the cells that were not pre-incubated with TUDCA and PB, the formazan absorbance in the presence of TUDCA and PB was higher than 75% of the control value in all groups, indicating that TUDCA and PB did not affect the viability of HEK cells at the tested concentrations (Table 49 and Table 50).

In the subsequent assays, the highest concentration of TUDCA did not exceed 348 μM in HBSSg_7.4 and 810 μM in HBSSg_8.5 in these cells, and, for PB, 32625 μM in HBSSg_7.4 and 73927 μM in HBSSg_8.5.

TABLE 48
Effects of TUDCA and PB on Viability of VC-Transfected HEK Cells
(Preincubation with the Test Article)

| | | | Replicate | | | Avg. Net |
|---|---|---|---|---|---|---|
| Cell Line | Treatment^{a} | Parameter | 1 | 2 | 3 | Absorbance |
| Vector | HBSSg_7.4 only | Net absorbance | 0.442 | 0.406 | 0.427 | 0.425 |
| control- | 174 μM TUDCA | Net absorbance | 0.450 | 0.433 | 0.449 | ND |
| transfected | 16312 μM PB | % of HBSSg_7.4^{b} | 106 | 102 | 106 | |
| cells | 348 μM TUDCA | Net absorbance | 0.429 | 0.425 | 0.424 | |
| | and 32625 μM PB | % of HBSSg_7.4^{b} | 101 | 100 | 99.8 | |

^{a}The cells were pre-incubated with test article solution.
^{b}The percentage was obtained by dividing the individual absorbance of each well in the presence of the test article by the average value in the absence of the test article.
ND: not determined.

TABLE 49
Effects of TUDCA and PB on Viability of VC-Transfected HEK Cells
(No Pre-incubation with the Test Article)

| | | | Replicate | | | Avg. Net |
|---|---|---|---|---|---|---|
| Cell Line | Treatment^{a} | Parameter | 1 | 2 | 3 | Absorbance |
| Vector | HBSSg_7.4 only | Net absorbance | 0.621 | 0.564 | 0.564 | 0.583 |
| control- | 174 μM TUDCA | Net absorbance | 0.573 | 0.561 | 0.550 | ND |
| transfected | 16312 μM PB | % of HBSSg_7.4^{b} | 98.3 | 96.2 | 94.3 | |
| cells | 348 μM TUDCA | Net absorbance | 0.574 | 0.543 | 0.568 | |
| | and 32625 μM PB | % of HBSSg_7.4^{b} | 98.5 | 93.1 | 97.4 | |

^{a}The cells were pre-incubated with test article solution.
^{b}The percentage was obtained by dividing the individual absorbance of each well in the presence of the test article by the average value in the absence of the test article.
ND: not determined.

TABLE 50
Effects of TUDCA and PB on Viability of VC-Transfected HEK Cells
(No Pre-incubation with Test Article, pH 8.50)

| | | | Replicate | | | Avg. Net |
|---|---|---|---|---|---|---|
| Cell Line | Treatment^{a} | Parameter | 1 | 2 | 3 | Absorbance |
| Vector | HBSSg_8.5 only | Net absorbance | 0.433 | 0.430 | 0.409 | 0.424 |
| control- | 405 μM TUDCA | Net absorbance | 0.451 | 0.462 | 0.475 | ND |
| transfected | 36964 μM PB | % of HBSSg_8.5^{b} | 106 | 109 | 112 | |
| cells | 810 μM TUDCA | Net absorbance | 0.437 | 0.448 | 0.459 | |
| | and 73927 μM PB | % of HBSSg_8.5^{b} | 103 | 106 | 108 | |

### Uptake Transporter Inhibitor Assessment

(i) OAT1

The measured concentrations of TUDCA and PB in the dosing solution in the OAT1 IC50 assessment were within 80-120% of the nominal value for all three replicates at all concentrations, indicating that the preparation of the test article dosing solution was acceptable. The detailed annotation of Solution 1 through Solution 8 is described in Table 28. The preparation of PAH dosing solutions was also acceptable.

In the absence of TUDCA and PB or probenecid, the influx rate ratio of PAH was 385 (OAT1 over vector control) (Table 51). The presence of probenecid caused 86.5% inhibition of PAH uptake in OAT1-transfected cells (Table 52). These results indicate the normal function of the test system.

In the presence of TUDCA and PB, the average influx rate of PAH via OAT1 ranged from 4.90 to 411 pmol/mg protein/minute (Table 51). AMX0035 (combination of TUDCA and PB) showed concentration-dependent inhibition of OAT1-mediated uptake of PAH.

Because of the simultaneous presence of TUDCA and PB in the dosing solutions in the OAT1 IC50 assessment, it is not possible to determine the IC50 for either component. The inhibition of OAT1 was 44.8% in the presence of Solution 8 (0.0741 μM TUDCA and 6.76 μM PB) and 65.2% in the presence of Solution 7 (0.222 μM TUDCA and 20.3 μM PB) (Table 52). Therefore, the estimated IC50 toward OAT1 would be between 0.0741 and 0.222 μM TUDCA, with 6.76 and 20.3 μM PB, respectively.

Currently, the FDA has the following ratio criteria for in vitro inhibitor assessment of a test article toward OAT1: if unbound [I]1/IC50 is less than 0.1, then an in vivo drug-drug interaction study is not needed. The unbound [I]1 of TUDCA and PB is 1.62 and 148 μM, respectively (Table 28). The ratio would be greater than 0.1 for TUDCA (1.62/[0.0741˜2.22]) and PB (148/[6.76˜20.3]). Since the ratio is greater than 0.1, an in vivo drug-drug interaction study with an OAT1 substrate is recommended for AMX0035.

TABLE 51
Uptake of PAH in the Absence and Presence of TUDCA
and PB or Probenecid (OAT1 IC50 Assessment)

| | | 5 | |
|---|---|---|---|
| | | Influx Rate (pmol/mg protein/minute) | |
| | Incubation | Vector | |
| | (minutes) | Control | OAT1-HEK |
| Treatment | Replicate | Cells | Cells |
| 10 μM PAH | 1 | 2.06 | 766 |
| | 2 | 1.79 | 720 |
| | Average | 1.93 | 743 |
| | Influx Rate | NA | 385 |
| | Ratio | | |
| 10 μM PAH + | 1 | 2.18 | 433 |
| Solution 8 | 2 | 2.38 | 390 |
| | Average | 2.28 | 411 |
| 10 μM PAH + | 1 | 1.90 | 282 |
| Solution 7 | 2 | 3.06 | 239 |
| | Average | 2.48 | 261 |
| 10 μM PAH + | 1 | 2.79 | 134 |
| Solution 6 | 2 | 3.42 | 119 |
| | Average | 3.10 | 126 |
| 10 μM PAH + | 1 | 2.36 | 63.2 |
| Solution 5 | 2 | 2.81 | 54.8 |
| | Average | 2.58 | 59.0 |
| 10 μM PAH + | 1 | 2.71 | 32.4 |
| Solution 4 | 2 | 2.28 | 29.8 |
| | Average | 2.49 | 31.1 |
| 10 μM PAH + | 1 | 2.79 | 16.8 |
| Solution 3 | 2 | 2.69 | 15.6 |
| | Average | 2.74 | 16.2 |
| 10 μM PAH + | 1 | 2.14 | 10.9 |
| Solution 2 | 2 | 1.80 | 12.3 |
| | Average | 1.97 | 11.6 |
| 10 μM PAH + | 1 | 1.45 | 4.36 |
| Solution 1 | 2 | 1.16 | 5.43 |
| | Average | 1.30 | 4.90 |
| 10 μM PAH + | 1 | 2.40 | 103 |
| 100 μM Probenecid | 2 | 2.06 | 101 |
| | Average | 2.23 | 102 |

TABLE 52
Net Influx Rate of PAH and Percentage Inhibition
of OAT1 by AMX0035 and Probenecid

| | OAT1 | |
|---|---|---|
| | Net Influx Rate | Percentage |
| Treatment | (pmol/mg protein/minute) | Inhibition |
| 10 μM PAH | 741 | NA |
| 10 μM PAH + Solution 1 | 3.59 | 99.5 |
| 10 μM PAH + Solution 2 | 9.64 | 98.7 |
| 10 μM PAH + Solution 3 | 13.5 | 98.2 |
| 10 μM PAH + Solution 4 | 28.6 | 96.1 |
| 10 μM PAH + Solution 5 | 56.4 | 92.4 |
| 10 μM PAH + Solution 6 | 123 | 83.4 |
| 10 μM PAH + Solution 7 | 258 | 65.2 |
| 10 μM PAH + Solution 8 | 409 | 44.8 |
| 10 μM PAH + 100 μM | 99.7 | 86.5 |
| Probenecid | | |

NA: not applicable

(ii) OCT2

The measured concentrations of TUDCA and PB in the dosing solution in the OCT2 IC50 assessment were within 80-120% of the nominal value for all three replicates at all concentrations, indicating that the preparation of the test article dosing solution was acceptable. The detailed annotation of Solution 1 through Solution 8 is described in Table 37. The preparation of MPP+ dosing solutions was also acceptable.

In the absence of TUDCA and PB or imipramine, the influx rate ratio of MPP+ was 48.9 (OCT2 over vector control) (Table 53). The presence of imipramine caused 97.9% inhibition of MPP+ uptake in OCT2-transfected cells (Table 54). These results indicate the normal function of the test system.

In the presence of TUDCA and PB, the average influx rate of MPP+ via OCT2 ranged from 680 to 991 pmol/mg protein/minute (Table 53). AMX0035 (combination of TUDCA and PB) did not inhibit OCT2-mediated uptake of MPP+ in the tested concentration range. Because the percentage inhibition was less than 50%, the estimated IC50 toward OCT2 would be greater than 162 μM and greater than 14785 μM, respectively.

TABLE 53
Uptake of MPP+ in the Absence and Presence
of TUDCA and PB or Imipramine (OCT2 IC50 Assessment)

| | | 2 | |
|---|---|---|---|
| | | Influx Rate (pmol/mg protein/minute) | |
| | Incubation | Vector | |
| | (minutes) | Control | OCT2-HEK |
| Treatment | Replicate | Cells | Cells |
| 5 μM MPP⁺ | 1 | 19.0 | 896 |
| | 2 | 16.3 | 833 |
| | Average | 17.7 | 864 |
| | Influx Rate | NA | 48.9 |
| | Ratio | | |
| 5 μM MPP⁺ + | 1 | 19.9 | 870 |
| Solution 8 | 2 | 21.1 | 791 |
| | Average | 20.5 | 830 |
| 5 μM MPP⁺ + | 1 | 24.7 | 812 |
| Solution 7 | 2 | 19.4 | 843 |
| | Average | 22.1 | 827 |
| 5 μM MPP⁺ + | 1 | 14.4 | 711 |
| Solution 6 | 2 | 13.5 | 648 |
| | Average | 14.0 | 680 |
| 5 μM MPP⁺ + | 1 | 15.7 | 806 |
| Solution 5 | 2 | 16.4 | 743 |
| | Average | 16.0 | 775 |
| 5 μM MPP⁺ + | 1 | 17.1 | 864 |
| Solution 4 | 2 | 17.2 | 738 |
| | Average | 17.2 | 801 |
| 5 μM MPP⁺ + | 1 | 15.7 | 928 |
| Solution 3 | 2 | 17.4 | 812 |
| | Average | 16.5 | 870 |
| 5 μM MPP⁺ + | 1 | 18.2 | 1043 |
| Solution 2 | 2 | 15.9 | 938 |
| | Average | 17.0 | 991 |
| 5 μM MPP⁺ + | 1 | 14.3 | 1017 |
| Solution 1 | 2 | 16.0 | 912 |
| | Average | 15.2 | 964 |
| 5 μM MPP⁺ + | 1 | 6.97 | 23.4 |
| 300 μM Imipramine | 2 | 7.75 | 26.2 |
| | Average | 7.36 | 24.8 |

TABLE 54
Net Influx Rate of MPP+ and Percentage
Inhibition of OCT2 by AMX0035 and Imipramine

| | OCT2 | |
|---|---|---|
| | Net Influx Rate | |
| | (pmol/mg | Percentage |
| Treatment | protein/minute) | Inhibition |
| 5 μM MPP⁺ | 847 | NA |
| 5 μM MPP⁺ + Solution 1 | 949 | 0 (−12.1)^{a} |
| 5 μM MPP⁺ + Solution 2 | 974 | 0 (−15.0)^{a} |
| 5 μM MPP⁺ + Solution 3 | 853 | 0 (−0.756)^{a} |
| 5 μM MPP⁺ + Solution 4 | 784 | 7.41 |
| 5 μM MPP⁺ + Solution 5 | 759 | 10.4 |
| 5 μM MPP⁺ + Solution 6 | 666 | 21.4 |
| 5 μM MPP⁺ + Solution 7 | 805 | 4.88 |
| 5 μM MPP⁺ + Solution 8 | 810 | 4.38 |
| 5 μM MPP⁺ + 300 μM Imipramine | 17.4 | 97.9 |

^{a}The negative percentage inhibition indicates no inhibition.

The criteria used to assess the in vitro inhibition of OCT2 is the same as for OAT1. The unbound [I]1 of TUDCA and PB is 1.62 and 148 μM, respectively (Table 28). The ratio would be less than 0.1 for TUDCA (1.62/[>162]) and PB (148/[>14785]). Because the ratio is less than 0.1, an in vivo drug-drug interaction study with an OCT2 substrate is not recommended for AMX0035.

(iii) OAT3

The measured concentrations of TUDCA and PB in the dosing solution in the OAT3 IC50 assessment were within 80-120% of the nominal value for all three replicates at all concentrations, indicating that the preparation of the test article dosing solution was acceptable. The detailed annotation of Solution 1 through Solution 8 is described in Table 37. The preparation of furosemide dosing solutions was also acceptable.

In the absence of TUDCA and PB or probenecid, the influx rate ratio of furosemide was 20.1 (OAT3 over vector control) (Table 55). The presence of probenecid caused 99.1% inhibition of furosemide uptake in OAT3-transfected cells (Table 56). These results indicate the normal function of the test system.

In the presence of TUDCA and PB, the average influx rate of furosemide via OAT3 ranged from 11.3 to 62.0 pmol/mg protein/minute (Table 55). AMX0035 (combination of TUDCA and PB) showed greater than 50% inhibition (80.3%) of OAT3-mediated uptake of furosemide at the highest tested concentration (Table 56).

TABLE 55
Uptake of Furosemide in the Absence and Presence of
TUDCA and PB or Probenecid (OAT3 IC50 Assessment)

| | | Influx Rate (pmol/mg protein/minute) | |
|---|---|---|---|
| | Incubation | Vector | |
| | (minutes) | Control | OAT3-HEK |
| Treatment | Replicate | Cells | Cells |
| 5 μM Furosemide | 1 | 2.47 | 42.2 |
| | 2 | 1.98 | 47.3 |
| | Average | 2.23 | 44.8 |
| | Influx Rate | NA | 20.1 |
| | Ratio | | |
| 5 μM Furosemide + | 1 | 1.99 | 39.1 |
| Solution 8 | 2 | 2.82 | 45.6 |
| | Average | 2.40 | 42.3 |
| 5 μM Furosemide + | 1 | 2.15 | 54.8 |
| Solution 7 | 2 | 2.80 | 47.4 |
| | Average | 2.48 | 51.1 |
| 5 μM Furosemide + | 1 | 3.41 | 66.6 |
| Solution 6 | 2 | 4.22 | 51.8 |
| | Average | 3.32 | 59.2 |
| 5 μM Furosemide + | 1 | 2.14 | 59.4 |
| Solution 5 | 2 | 4.49 | 58.4 |
| | Average | 3.32 | 58.9 |
| 5 μM Furosemide + | 1 | 3.18 | 66.1 |
| Solution 4 | 2 | 3.68 | 57.9 |
| | Average | 3.43 | 62.0 |
| 5 μM Furosemide + | 1 | 3.08 | 50.1 |
| Solution 3 | 2 | 3.81 | 48.4 |
| | Average | 3.45 | 46.7 |
| 5 μM Furosemide + | 1 | 4.87 | 25.9 |
| Solution 2 | 2 | 2.79 | 27.0 |
| | Average | 3.83 | 26.4 |
| 5 μM Furosemide + | 1 | 3.07 | 10.5 |
| Solution 1 | 2 | 2.77 | 12.1 |
| | Average | 2.92 | 11.3 |
| 5 μM Furosemide + | 1 | 2.97 | 3.85 |
| 100 μM Probenecid | 2 | 2.79 | 2.70 |
| | Average | 2.88 | 3.27 |

The inhibition of OAT3 was 46.8% in the presence of Solution 2 (54.0 μM TUDCA and 4928 μM PB) and 80.3% in the presence of Solution 1 (162 μM TUDCA and 14785 μM PB) (Table 56). Therefore, the estimated IC50 for OAT3 would be between 5.40 and 162 μM TUDCA, with 4928 and 14785 μM PB, respectively.

TABLE 56
Net Influx Rate of Furosemide and Percentage
Inhibition of OAT3 by AMX0035 and Probenecid

| | OAT3 | |
|---|---|---|
| | Net Influx Rate | Percentage |
| Treatment | (pmol/mg protein/minute) | Inhibition |
| 5 μM Furosemide | 42.5 | NA |
| 5 μM Furosemide + Solution 1 | 8.38 | 80.3 |
| 5 μM Furosemide + Solution 2 | 22.6 | 46.8 |
| 5 μM Furosemide + Solution 3 | 43.3 | 0(−1.71)ª |
| 5 μM Furosemide + Solution 4 | 58.6 | 0(−37.7)ª |
| 5 μM Furosemide + Solution 5 | 55.6 | 0(−30.7)ª |
| 5 μM Furosemide + Solution 6 | 55.9 | 0(−31.3)ª |
| 5 μM Furosemide + Solution 7 | 48.6 | 0(−14.3)ª |
| 5 μM Furosemide + Solution 8 | 39.9 | 6.12 |
| 5 μM Furosemide + 100 μM | 0.398 | 99.1 |
| Probenecid | | |

The criteria used to assess the in vitro inhibition of OAT3 is the same as for OAT1. The unbound [I]1 of TUDCA and PB is 1.62 and 148 μM, respectively (Table 28). The ratio would be less than 0.1 for TUDCA (1.62/[54.0-162]) and PB (148/[4928-14785]). Because the ratio is less than 0.1, an in vivo drug-drug interaction study with an OAT3 substrate is not recommended for AMX0035.

(iv) OATP1B1 and OATP1B3

The measured concentrations of TUDCA and PB in the dosing solution in the OATP1B1 and OATP1B3 IC50 assessment were within 80-120% of the nominal value for at least two of three replicates at all concentrations, indicating that the preparation of the test article dosing solution was acceptable. The detailed annotation of Solution 1 through Solution 8 is described in Table 37. The preparation of atorvastatin dosing solutions was also acceptable.

In the absence of TUDCA and PB or rifamycin SV (RSV), the influx rate ratios of atorvastatin were 15.1 (OATP1B1 over vector control) and 8.23 (OATP1B3 over vector control) (Table 57). The presence RSV caused 96.7% and 98.1% inhibition of atorvastatin uptake in OATP1B1- and OATP1B3-transfected cells, respectively (Table 58). These results indicate the normal function of the test systems.

In the presence of TUDCA and PB, the average influx rate of atorvastatin ranged from 0.660 to 6.26 pmol/mg protein/minute via OATP1B 1, and from 0.813 to 2.93 pmol/mg protein/minute via OATP1B3 (Table 57).

TABLE 57
Uptake of Atorvastatin in the Absence and Presence of TUDCA
and PB or Rifamycin SV (OATP1B1 and OATP1B3 IC50 Assessment)

| | | 5 | | |
|---|---|---|---|---|
| | | Influx Rate (pmol/mg protein/minute) | | |
| | Incubation | Vector | | |
| | (minutes) | Control | OATP1B1- | OATP1B3- |
| Treatment | Replicate | Cells | HEK Cells | HEK Cells |
| 0.15 μM | 1 | 0.365 | 5.89 | 3.14 |
| Atorvastatin | 2 | 0.421 | 5.94 | 3.34 |
| | Average | 0.393 | 5.92 | 3.24 |
| | Influx Rate | NA | 15.1 | 8.23 |
| | Ratio | | | |
| 0.15 μM | 1 | 0.379 | 6.14 | 2.51 |
| Atorvastatin + | 2 | 0.377 | 6.39 | 2.59 |
| Solution 8 | Average | 0.378 | 6.26 | 2.55 |
| 0.15 μM | 1 | 0.452 | 6.03 | 2.57 |
| Atorvastatin + | 2 | 0.408 | 5.83 | 2.54 |
| Solution 7 | Average | 0.430 | 5.93 | 2.55 |
| 0.15 μM | 1 | 0.367 | 5.56 | 2.60 |
| Atorvastatin + | 2 | 0.348 | 5.67 | 2.54 |
| Solution 6 | Average | 0.357 | 5.61 | 2.57 |
| 0.15 μM | 1 | 0.355 | 4.83 | 2.81 |
| Atorvastatin + | 2 | 0.357 | 5.02 | 2.88 |
| Solution 5 | Average | 0.356 | 4.93 | 2.85 |
| 0.15 μM | 1 | 0.372 | 3.68 | 2.94 |
| Atorvastatin + | 2 | 0.379 | 3.82 | 2.92 |
| Solution 4 | Average | 0.375 | 3.75 | 2.93 |
| 0.15 μM | 1 | 0.393 | 2.27 | 2.33 |
| Atorvastatin + | 2 | 0.378 | 2.36 | 2.19 |
| Solution 3 | Average | 0.386 | 2.31 | 2.26 |
| 0.15 μM | 1 | 0.387 | 1.06 | 1.43 |
| Atorvastatin + | 2 | 0.305 | 1.03 | 1.31 |
| Solution 2 | Average | 0.346 | 1.05 | 1.37 |
| 0.18 μM | 1 | 0.461 | 0.651 | 0.787 |
| Atorvastatin + | 2 | 0.421 | 0.669 | 0.840 |
| Solution 1 | Average | 0.441 | 0.660 | 0.813 |
| 0.15 μM | 1 | 0.496 | 0.724 | 0.576 |
| Atorvastatin + | 2 | 0.541 | 0.681 | 0.571 |
| 10 μM RSV | Average | 0.518 | 0.702 | 0.573 |

TABLE 58
Net Influx Rate of Atorvastatin and Percentage Inhibition
of AMX0035 and RSV toward OATP1B1 and OATP1B3

| | OATP1B1 | | OATP1B3 | |
|---|---|---|---|---|
| | Net Influx Rate | Percentage | Net Influx Rate | Percentage |
| Treatment | (pmol/mg protein/minute) | Inhibition | (pmol/mgprotein/minute) | Inhibition |
| 0.15 μM Atorvastatin | 5.52 | 0 | 2.84 | NA |
| 0.15 μM Atorvastatin + Solution 1 | 0.219 | 96.0 | 0.372 | 86.9 |
| 0.15 μM Atorvastatin + Solution 2 | 0.700 | 87.3 | 1.02 | 64.0 |
| 0.15 μM Atorvastatin + Solution 3 | 1.93 | 65.1 | 1.87 | 34.1 |
| 0.15 μM Atorvastatin + Solution 4 | 3.37 | 38.9 | 2.55 | 10.1 |
| 0.15 μM Atorvastatin + Solution 5 | 4.57 | 17.3 | 2.49 | 12.4 |
| 0.15 μM Atorvastatin + Solution 6 | 5.26 | 4.85 | 2.21 | 22.1 |
| 0.15 μM Atorvastatin + Solution 7 | 5.50 | 0.392 | 2.12 | 25.4 |
| 0.15 μM Atorvastatin + Solution 8 | 5.89 | 0(−6.55)^{a} | 2.17 | 23.6 |
| 0.15 μM Atorvastatin + 10 μM RSV | 0.184 | 96.7 | 0.0551 | 98.1 |

The inhibition of OATP1B1 was 38.9% in the presence of Solution 4 (12.9 μM TUDCA and 1208 μM PB) and 65.1% in the presence of Solution 3 (38.7 μM TUDCA and 3625 μM PB) (Table 58). Therefore, the estimated IC50 toward OATP1B1 would be between 12.9 and 38.7 μM TUDCA, with 1208 and 3625 μM PB, respectively. Similarly, the estimated IC50 toward OATP1B3 would be between 38.7 and 116 μM TUDCA, with 3625 and 10875 μM PB, respectively.

Currently, the FDA uses unbound maximal plasma inhibitor concentration at the inlet to the liver (unbound I_{in,max}) with oral administration. If the ratio of unbound I_{in,max} over IC50 is greater than 0.1, the test article has the potential to inhibit OATP1B1/3. The projected unbound I_{in,max} of TUDCA and PB is 3.48 and 326 μM, respectively (Table 28).

For OATP1B1, the ratio for TUDCA would be [3.48/(12.9˜38.7)] and [326/(1208˜3625)] for PB. Based on the current data, because the IC50 cannot be estimated more precisely than a range of concentrations, it is not conclusive whether the ratio would be greater than 0.1.

For OATP1B3, the ratio would be less than 0.1 for TUDCA [3.48/(38.7˜116)] and PB [326/(3625˜10875)]. Because the ratio is less than 0.1, an in vivo drug-drug interaction study with an OATP1B3 substrate is not recommended for AMX0035.

(v) MATE1 and MATE2K

The measured concentrations of TUDCA and PB in the dosing solution in the MATE1 and MATE2K IC50 assessment were within 80-120% of the nominal value for at least two of three replicates at all concentrations, indicating that the preparation of the test article dosing solution was acceptable. The detailed annotation of Solution 1 through Solution 8 is described in Table 37. The preparation of metformin dosing solutions was also acceptable.

In the absence of TUDCA and PB or positive inhibitor, the influx rate ratios of metformin were 43.4 (MATE1 over vector control, Table 59) and 2.70 (MATE2K over vector control, Table 60). The presence of cimetidine or pyrimethamine caused 99.2% and 85.3% inhibition of metformin uptake in MATE1- and MATE2K-transfected cells, respectively (Table 61). These results indicate the normal function of the test systems.

In the presence of TUDCA and PB, the average influx rate of metformin ranged from 189 to 435 pmol/mg protein/minute via MATE1 (Table 59) and from 36.7 to 52.0 pmol/mg protein/minute via MATE2K (Table 60).

TABLE 59
Uptake of Metformin in the Absence and Presence of
TUDCA and PB or Cimetidine (MATE1 IC50 Assessment)

| | | 10 | |
|---|---|---|---|
| | | Influx Rate (pmol/mg protein/minute) | |
| | Incubation | Vector | |
| | (minutes) | Control | MATE1-HEK |
| Treatment | Replicate | Cells | Cells |
| 50 μM Metformin | 1 | 15.6 | 515 |
| | 2 | 11.3 | 656 |
| | Average | 13.5 | 586 |
| | Influx Rate | NA | 43.4 |
| | Ratio | | |
| 50 μM Metformin + | 1 | 9.62 | 393 |
| Solution 8 | 2 | 13.7 | 477 |
| | Average | 11.7 | 435 |
| 50 μM Metformin + | 3 | 11.8 | 346 |
| Solution 7 | 2 | 13.8 | 435 |
| | Average | 12.8 | 390 |
| 50 μM Metformin + | 1 | 11.0 | 338 |
| Solution 6 | 2 | 13.0 | 376 |
| | Average | 12.0 | 357 |
| 50 μM Metformin + | 1 | 10.9 | 348 |
| Solution 5 | 2 | 12.0 | 423 |
| | Average | 11.4 | 386 |
| 50 μM Metformin + | 1 | 14.0 | 343 |
| Solution 4 | 2 | 13.4 | 423 |
| | Average | 13.7 | 383 |
| 50 μM Metformin + | 1 | 10.7 | 374 |
| Solution 3 | 2 | 11.9 | 464 |
| | Average | 11.3 | 419 |
| 50 μM Metformin + | 1 | 14.6 | 335 |
| Solution 2 | 2 | 10.7 | 406 |
| | Average | 12.7 | 371 |
| 50 μM Metformin + | 1 | 12.9 | 168 |
| Solution 1 | 2 | 8.78 | 210 |
| | Average | 10.9 | 189 |
| 50 μM Metformin + | 1 | 13.2 | 14.7 |
| 100 μM Cimetidine | 2 | 8.43 | 16.1 |
| | Average | 10.8 | 15.4 |

The inhibition of MATE1 was 37.500 in the presence of Solution 2 (270 μM TUDCA and 24642 μM PB) and 68.9% in the presence of Solution 1 (810 μM TUDCA and 73927 μM PB) (Table 61). Therefore, the estimated IC50 toward MATE1 would be between 270 and 810 μM TUDCA, with 24642 and 73927 μM PB, respectively. Similarly, the estimated IC50 of TUDCA and PB toward MATE2K would be greater than 810 μM and greater than 73927 μM, respectively.

TABLE 60
Uptake of Metformin in the Absence and Presence of
TUDCA and PB or Cimetidine (MATE2K IC50 Assessment)

10
Influx Rate (pmol/mg
protein/minute)

| | Incubation | Vector | MATE2K |
|---|---|---|---|
| | (minutes) | Control | HEK |
| Treatment | Replicate | Cells | Cells |
| 50 μM Metformin | 1 | 19.6 | 54.5 |
| | 2 | 22.8 | 60.2 |
| | Average | 21.3 | 57.4 |
| | Influx Rate | NA | 2.70 |
| | Ratio | | |
| 50 μM Metformin + | 1 | 7.84 | 44.7 |
| Solution 8 | 2 | 8.48 | 42.0 |
| | Average | 8.16 | 43.4 |
| 50 μM Metformin + | 1 | 10.9 | 39.3 |
| Solution 7 | 2 | 10.9 | 43.0 |
| | Average | 10.0 | 41.2 |
| 50 μM Metformin + | 1 | 8.68 | 43.1 |
| Solution 6 | 2 | 9.22 | 41.9 |
| | Average | 8.95 | 42.5 |
| 50 μM Metformin + | 1 | 8.58 | 40.2 |
| Solution 5 | 2 | 12.8 | 45.6 |
| | Average | 10.7 | 42.9 |
| 50 μM Metformin + | 1 | 10.2 | 37.7 |
| Solution 4 | 2 | 10.9 | 43.4 |
| | Average | 10.6 | 40.6 |
| 50 μM Metformin + | 1 | 7.40 | 43.4 |
| Solution 3 | 2 | 11.2 | 43.2 |
| | Average | 9.30 | 43.3 |
| 50 μM Metformin + | 1 | 11.4 | 54.0 |
| Solution 2 | 2 | 12.5 | 50.1 |
| | Average | 12.0 | 52.0 |
| 50 μM Metformin + | 1 | 13.5 | 39.9 |
| Solution 1 | 2 | 16.4 | 33.5 |
| | Average | 14.9 | 36.7 |
| 50 μM Metformin + | 1 | 14.5 | 20.2 |
| 1 μM Pyrimethamine | 2 | 13.0 | 17.9 |
| | Average | 13.7 | 19.0 |

TABLE 61
Net Influx Rate of Metformin, Percentage Inhibition, and IC50 of TUDCA and PB toward MATE1 and MATE2K

| | MATE1 | | MATE2K | |
|---|---|---|---|---|
| | Net Influx Rate | Percentage | Net Influx Rate | Percentage |
| Treatment | (pmol/mg protein/minute) | Inhibition | (pmol/mg protein/minute) | Inhibition |
| 50 μM Metformin | 572 | 0 | 36.1 | NA |
| 50 μM Metformin + Solution 1 | 178 | 68.9 | 21.8 | 39.7 |
| 50 μM Metformin + Solution 2 | 358 | 37.5 | 40.1 | 0 (−11.0)^{a} |
| 50 μM Metformin + Solution 3 | 408 | 28.7 | 34.0 | 5.83 |
| 50 μM Metformin + Solution 4 | 369 | 35.5 | 30.0 | 16.9 |
| 50 μM Metformin + Solution 5 | 374 | 34.6 | 32.2 | 10.8 |
| 50 μM Metformin + Solution 6 | 345 | 39.7 | 33.6 | 7.08 |
| 50 μM Metformin + Solution 7 | 378 | 34.0 | 30.2 | 16.4 |
| 50 μM Metformin + Solution 8 | 423 | 26.0 | 35.2 | 2.50 |
| 50 μM Metformin + 100 μM Cimetidine | 4.57 | 99.2 | NA | NA |
| 50 μM Metformin + 1 μM Pyrimethamine | NA | NA | 5.31 | 85.3 |

Currently, the FDA has the following ratio criteria for in vitro inhibitor assessment of a test article toward MATE1 and MATE2K: if the ratio of unbound [I]1/IC50 is greater than 0.02, the test article has the potential to inhibit MATE1/2K. The projected unbound C max of TUDCA and PB is 1.62 SM and 148 μM, respectively (Table 28).

For MATE1, the ratio would be less than 0.02 for TUDCA [1.62/(270˜10)] and PB [148/(2464273927)]. Similarly, the ratios would be less than 0.02 for MATE2K. Since the ratios are less than 0.02, an in vivo drug-drug interaction study with a MATE1 or MATE2K substrate is not needed for AMVX0035.

### P-gp and BCRP IC50Assessment

(i) P-gp IC₅₀ Assessment

The concentrations of TUDCA and PB in the dosing solutions for the P-gp IC50 assessment in MIDR1-MDCK cells were determined. The measured concentrations of each component in at least two of three replicates were in the range of 8010 of the nominal value in each case, indicating that the preparation of test article dosing solutions was acceptable. The detailed annotation of Solution 1 through Solution 8 is described in Table 39. The preparation of digoxin was also acceptable in all but in the presence of Solution 2.

In MDR1-MDCK cells, in the absence of TUDCA and PB or valspodar, the efflux ratio of digoxin was 304, and the addition of valspodar decreased it to 1.23 (Table 62), corresponding to 99.9% inhibition (Table 63). These results indicate the normal function of P-gp in the test system.

In the presence of TUDCA and PB, the efflux ratio of digoxin ranged from 60.4 to 270 (Table 62). The inhibition of P-gp was 80.4% (greater than 50%) in the presence of Solution 2 (2668 μM TUDCA and 21482 μM PB), but was variable at lower concentration (Table 63). Such variability was due to cell-cell variability rather than P-gp inhibition by TUDCA and PB. Therefore, the estimated IC50 toward P-gp would be between 889 and 2668 μM TUDCA, with 7161 and 21482 μM PB, respectively.

Currently, for orally administered drugs, the FDA uses the following ratio to assess the in vitro P-gp inhibitor potential: [I]₂ of the test article over its calculated IC₅₀. Correspondingly, if [I]₂ over IC₅₀ is less than 10, an in vivo drug-drug interaction study with a P-gp substrate is not needed.

The estimated [I]₂ of TUDCA and PB is 8005 and 64447 μM (Table 28). The projected ratio is less than 10 [8005/(889˜2668)] for TUDCA and less than 10 [64447/(7161-21482)] for PB in MDR1-MDCK cells.

TABLE 62
Permeability and Recovery of Digoxin in the Absence and
Presence of TUDCA and PB or Valspodar in MDR1-MDCK Cells

| | | AP-to-BL | | BL-to-AP | | |
|---|---|---|---|---|---|---|
| | | Pₐₚₚ | Recov- | Pₐₚₚ | Recov- | |
| | | (10⁻⁶ | ery | (10⁻⁶ | ery | Efflux |
| Treatment | Replicate | cm/s)^{a} | (%) | cm/s)^{a} | (%) | Ratio |
| 10 μM | 1 | 0.0343 | 73.7 | 9.97 | 83.5 | 304 |
| Digoxin | 2 | 0.0314 | 84.4 | 9.97 | 93.8 | |
| | Average | 0.0328 | 79.1 | 9.97 | 88.7 | |
| 10 μM | 1 | 0.0403 | 75.4 | 6.72 | 77.2 | 182 |
| Digoxin + | 2 | 0.0379 | 76.6 | 7.51 | 86.1 | |
| Solution 8 | Average | 0.0391 | 76.0 | 7.12 | 81.7 | |
| 10 μM | 1 | 0.0289 | 75.5 | 7.77 | 87.2 | 270 |
| Digoxin + | 2 | 0.0273 | 78.5 | 7.44 | 83.9 | |
| Solution 7 | Average | 0.0281 | 77.0 | 7.61 | 85.5 | |
| 10 μM | 1 | 0.0278 | 77.4 | 7.03 | 79.3 | 253 |
| Digoxin + | 2 | 0.0362 | 80.0 | 9.16 | 87.9 | |
| Solution 6 | Average | 0.0320 | 78.7 | 8.10 | 83.6 | |
| 10 μM | 1 | 0.0482 | 78.2 | 10.6 | 86.5 | 258 |
| Digoxin + | 2 | 0.0379 | 83.3 | 11.6 | 90.4 | |
| Solution 5 | Average | 0.0431 | 80.7 | 11.1 | 88.5 | |
| 10 μM | 1 | 0.0633 | 85.7 | 10.7 | 89.4 | 181 |
| Digoxin + | 2 | 0.0526 | 85.5 | 10.2 | 99.3 | |
| Solution 4 | Average | 0.0579 | 85.6 | 10.5 | 94.4 | |
| 10 μM | 1 | 0.0699 | 99.3 | 14.6 | 99.1 | 207 |
| Digoxin + | 2 | 0.0676 | 100 | 13.9 | 107 | |
| Solution 3 | Average | 0.0688 | 99.7 | 14.2 | 103 | |
| 10 μM | 1 | 0.140 | 110 | 7.93 | 111 | 60.4 |
| Digoxin + | 2 | 0.125 | 112 | 8.12 | 126 | |
| Solution 2 | Average | 0.133 | 111 | 8.03 | 118 | |
| 10 μM | 1 | 0.235^{b} | 117 | 3.77^{b} | 120 | NA |
| Digoxin + | 2 | 0.248^{b} | 122 | 3.61^{b} | 122 | |
| Solution 1 | Average | NA | 119 | NA | 121 | |
| 10 μM | 1 | 0.249 | 79.4 | 0.345 | 84.7 | 1.23 |
| Digoxin + | 2 | 0.276 | 81.8 | 0.299 | 92.5 | |
| 1 μM | Average | 0.263 | 80.6 | 0.322 | 88.6 | |
| Valspodar | | | | | | |

^{a}The Pₐₚₚ values were calculated with Equation (7 − C).
^{b}The Pₐₚₚ of LY was greater than 0.8 × 10⁻⁶ cm/s, suggesting compromised cell monolayer integrity. The Pₐₚₚ of digoxin was excluded from the calculation of the average.
NA: not applicable.

TABLE 63
Corrected Efflux Ratio of Digoxin and Inhibition
by TUDCA, PB, and Valspodar (MDR1-MDCK Cells)

| | Corrected Efflux | Percent |
|---|---|---|
| Treatment | Ratio | Inhibition |
| 10 μM Digoxin | 303 | NA |
| 10 μM Digoxin + Solution 1 | NA | NA |
| 10 μM Digoxin + Solution 2 | 59.4 | 80.4 |
| 10 μM Digoxin + Solution 3 | 206 | 32.0 |
| 10 μM Digoxin + Solution 4 | 180 | 40.6 |
| 10 μM Digoxin + Solution 5 | 257 | 15.1 |
| 10 μM Digoxin + Solution 6 | 252 | 16.7 |
| 10 μM Digoxin + Solution 7 | 269 | 11.0 |
| 10 μM Digoxin + Solution 8 | 181 | 40.2 |
| 10 μM Digoxin + 1 μM Valspodar | 0.226 | 99.9 |

NA: not applicable.

The concentrations of TUDCA and PB in the dosing solutions for the P-gp IC50 assessment in C2BBe1 cells were determined. The measured concentrations of each component in at least two of three replicates were in the range of 80-120% of the nominal value in each case, indicating that the preparation of test article dosing solutions was acceptable. The detailed annotation of Solution 1 through Solution 8 is described in Table 39. The preparation of digoxin was also acceptable.

In C2BBe1 cells, in the absence of TUDCA and PB or valspodar, the efflux ratio of digoxin was 20.5, and the addition of valspodar decreased it to 1.14 (Table 64), corresponding to 99.3% inhibition (Table 65). These results indicate the normal function of P-gp in the test system.

In the presence of TUDCA and PB, the efflux ratio of digoxin ranged from 2.80 to 18.9 (Table 64). The inhibition of P-gp was 69.2% (greater than 50%) in the presence of Solution 2 (667 μM TUDCA and 5371 μM PB), and it was 40.0% in the presence of Solution 3 (222 μM TUDCA and 1790 μM PB) (Table 65). Therefore, the estimated IC50 toward P-gp would be between 222 and 667 μM TUDCA, with 1790 and 5371 μM PB, respectively.

Currently, for orally administered drugs, the FDA uses the following ratio to assess the in vitro P-gp inhibitor potential: [I]₂ of the test article over its calculated IC₅₀. Correspondingly, if [I]₂ over IC₅₀ is less than 10, an in vivo drug-drug interaction study with a P-gp substrate is not needed.

The estimated [I]₂ of TUDCA and PB is 8005 and 64447 μM (Table 28). The projected ratio is greater than 10 [8005/(222˜667)] for TUDCA and greater than 10 [64447/(1790˜5371)] for PB in C2BBE1 cells.

Based on the current results, an in vivo drug-drug interaction of AMX0035 with a P-gp substrate may be needed.

(ii) BCRP IC₅₀ Assessment

The concentrations of TUDCA and PB in the dosing solutions for the BCRP IC50 assessment in BCRP-MDCK cells were determined. The measured concentrations of each component in at least two of three replicates were in the range of 80-120% of the nominal value in each case, indicating that the preparation of test article dosing solutions was acceptable. The detailed annotation of Solution 1 through Solution 8 is described in Table 39. The preparation of cladribine was also acceptable.

In the absence of TUDCA and PB or Ko143, the efflux ratio of cladribine was 223, and the addition of Ko143 decreased it to 0.922 (Table 66), corresponding to 100% inhibition (Table 67). These results indicate the normal function of BCRP in the test system.

In the presence of TUDCA and PB, the efflux ratio of cladribine ranged from 4.94 to 264 (Table 66). The inhibition of BCRP was 28.2% in the presence of Solution 4 (296 μM TUDCA and 2387 μM PB), and it was 90.7% in the presence of Solution 3 (889 μM TUDCA and 7161 M PB) (Table 67). Therefore, the estimated IC50 toward BCRP would be between 296 and 889 M TUDCA, with 2387 and 7161 μM PB, respectively.

Currently, for orally administered drugs, the FDA use the following ratio to assess the in vitro BCRP inhibitor potential: [I]₂ of the test article over its calculated IC₅₀. Correspondingly, if [I]₂ over IC₅₀ is less than 10, an in vivo drug-drug interaction study with a BCRP substrate is not needed.

The estimated [I]₂ of TUDCA and PB is 8005 and 64447 μM (Table 28). The projected ratio is [8005/(296˜889)] for TUDCA and [64447/(2387˜7161)] for PB in BCRP-MDCK cells. Based on the current data, because the IC50 cannot be estimated more precisely than a range of concentrations, it is not conclusive whether the ratio would be less than 10. A definitive in vitro-in vivo extrapolation is not available for BCRP inhibition using BCRP-MDCK cells.

TABLE 64
Permeability and Recovery of Digoxin in the Absence and
Presence of TUDCA and PB or Valspodar in C2BBe1 Cells

| | | AP-to-BL | | BL-to-AP | | |
|---|---|---|---|---|---|---|
| | | Pₐₚₚ | Recov- | Pₐₚₚ | Recov- | |
| | | (10⁻⁶ | ery | (10⁻⁶ | ery | Efflux |
| Treatment | Replicate | cm/s)^{a} | (%) | cm/s)^{a} | (%) | Ratio |
| 10 μM | 1 | 1.52^{b} | 75.5 | 10.2 | 81.7 | 20.5 |
| Digoxin | 2 | 0.501 | 82.9 | 10.3 | 86.7 | |
| | Average | NA | 79.2 | 10.3 | 84.2 | |
| 10 μM | 1 | 0.540 | 73.8 | 9.36 | 79.9 | 18.3 |
| Digoxin + | 2 | 0.583 | 87.8 | 11.2 | 82.3 | |
| Solution 8 | Average | 0.561 | 80.8 | 10.3 | 81.1 | |
| 10 μM | 1 | 0.618 | 71.1 | 8.93 | 74.9 | 14.3 |
| Digoxin + | 2 | 0.653 | 80.7 | 9.23 | 80.2 | |
| Solution 7 | Average | 0.635 | 75.9 | 9.08 | 77.6 | |
| 10 μM | 1 | 0.651 | 72.1 | 10.7 | 79.8 | 18.7 |
| Digoxin + | 2 | 0.539 | 86.6 | 11.5 | 85.7 | |
| Solution 6 | Average | 0.595 | 79.4 | 11.1 | 82.8 | |
| 10 μM | 1 | 0.715 | 70.2 | 13.6 | 77.1 | 18.9 |
| Digoxin + | 2 | 0.791 | 76.3 | 14.9 | 76.1 | |
| Solution 5 | Average | 0.753 | 73.2 | 14.2 | 76.6 | |
| 10 μM | 1 | 0.771 | 73.8 | 10.9 | 73.7 | 13.1 |
| Digoxin + | 2 | 1.13 | 78.7 | 13.9 | 87.1 | |
| Solution 4 | Average | 0.950 | 76.3 | 12.4 | 80.4 | |
| 10 μM | 1 | 1.05 | 78.7 | 14.0 | 83.1 | 12.7 |
| Digoxin + | 2 | 1.18 | 80.4 | 14.2 | 93.2 | |
| Solution 3 | Average | 1.11 | 79.5 | 14.1 | 88.1 | |
| 10 μM | 1 | 1.50 | 78.0 | 9.68 | 87.8 | 7.01 |
| Digoxin + | 2 | 1.69 | 88.3 | 12.7 | 97.5 | |
| Solution 2 | Average | 1.60 | 83.2 | 11.2 | 92.7 | |
| 10 μM | 1 | 1.65 | 90.2 | 4.29 | 93.3 | 2.80 |
| Digoxin + | 2 | 1.66 | 90.2 | 4.98 | 106 | |
| Solution 1 | Average | 1.65 | 90.2 | 4.64 | 99.9 | |
| 10 μM | 1 | 2.36 | 72.5 | 3.06 | 80.4 | 1.14 |
| Digoxin + | 2 | 2.37 | 77.7 | 2.35 | 85.1 | |
| 1 μM | Avenge | 2.37 | 75.1 | 2.71 | 82.7 | |
| Valspodar | | | | | | |

^{a}The Pₐₚₚ values were calculated with Equation (7 − C).
^{b}The Pₐₚₚ of LY was greater than 0.8 × 10⁻⁶ cm/s, suggesting compromised cell monolayer integrity. The Pₐₚₚ of digoxin was excluded from the calculation of the average.
NA: not applicable.

TABLE 65
Corrected Efflux Ratio of Digoxin and Inhibition
by TUDCA and PB and Valspodar (C2BBE1 Cells)

| | Corrected Efflux | Percent |
|---|---|---|
| Treatment | Ratio | Inhibition |
| 10 μM Digoxin | 19.5 | 0 |
| 10 μM Digoxin + Solution 1 | 1.80 | 90.8 |
| 10 μM Digoxin + Solution 2 | 6.01 | 69.2 |
| 10 μM Digoxin + Solution 3 | 11.7 | 40.0 |
| 10 μM Digoxin + Solution 4 | 12.1 | 38.2 |
| 10 μM Digoxin + Solution 5 | 17.9 | 8.27 |
| 10 μM Digoxin + Solution 6 | 17.7 | 9.11 |
| 10 μM Digoxin + Solution 7 | 13.3 | 31.8 |
| 10 μM Digoxin + Solution 8 | 17.3 | 11.4 |
| 10 μM Digoxin + 1 μM Valspodar | 0.144 | 99.3 |

TABLE 66
Permeability and Recovery of Cladribine in
the Absence and Presence of TUDCA and PB or
Ko143 in BCRP-MDCK Cells (BCRP-MDCK Cells)

| | | AP-to-BL | | BL-to-AP | | |
|---|---|---|---|---|---|---|
| | | Pₐₚₚ | Recov- | Pₐₚₚ | Recov- | |
| | | (10⁻⁶ | ery | (10⁻⁶ | ery | Efflux |
| Treatment | Replicate | cm/s)^{a} | (%) | cm/s)^{a} | (%) | Ratio |
| 10 μM | 1 | 0.0762 | 111 | 15.5 | 116 | 223 |
| Cladribine | 2 | 0.0577 | 128 | 14.3 | 124 | |
| | Average | 0.0670 | 120 | 14.9 | 120 | |
| 10 μM | 1 | 0.0909 | 103 | 17.6 | 96.9 | 199 |
| Cladribine + | 2 | 0.0865 | 94.6 | 17.7 | 114 | |
| Solution 8 | Average | 0.0887 | 98.9 | 17.6 | 105 | |
| 10 μM | 1 | 0.0805 | 96.7 | 15.9 | 85.0 | 236 |
| Cladribine + | 2 | 0.0600 | 96.5 | 17.2 | 102 | |
| Solution 7 | Average | 0.0703 | 96.6 | 16.6 | 93.6 | |
| 10 μM | 1 | 0.0831 | 100 | 15.3 | 89.3 | 264 |
| Cladribine + | 2 | 0.0443 | 91.5 | 18.4 | 100 | |
| Solution 6 | Average | 0.0637 | 95.7 | 16.8 | 94.5 | |
| 10 μM | 1 | 0.0823 | 93.0 | 14.0 | 90.2 | 149 |
| Cladribine + | 2 | 0.0893 | 88.1 | 11.6 | 98.1 | |
| Solution 5 | Average | 0.0858 | 90.6 | 12.8 | 94.2 | |
| 10 μM | 1 | 0.0617 | 106 | 8.66 | 75.1 | 160 |
| Cladribine + | 2 | 0.0432 | 71.6 | 8.15 | 101 | |
| Solution 4 | Average | 0.0525 | 88.8 | 8.40 | 88.2 | |
| 10 μM | 1 | 0.183 | 95.5 | 3.26 | 90.2 | 21.7 |
| Cladnibine + | 2 | 0.151 | 90.1 | 3.99 | 86.7 | |
| Solution 3 | Average | 0.167 | 92.8 | 3.63 | 88.5 | |
| 10 μM | 1 | 0.228 | 89.8 | 1.11 | 95.6 | 4.94 |
| Cladribine + | 2 | 0.205 | 97.0 | 1.03 | 90.3 | |
| Solution 2 | Average | 0.216 | 93.4 | 1.07 | 93.0 | |
| 10 μM | 1 | 0.863^{b} | 92.9 | 1.13^{b} | 89.9 | NA |
| Cladribine + | 2 | 0.692^{b} | 86.6 | 1.56^{b} | 94.8 | |
| Solution 1 | Average | NA | 89.8 | NA | 92.3 | |
| 10 μM | 1 | 0.155 | 80.1 | 0.135 | 95.4 | 0.922 |
| Cladribine + | 2 | 0.143 | 95.1 | 0.140 | 98.1 | |
| 0.5 μM | Average | 0.149 | 87.6 | 0.137 | 96.7 | |
| Ko143 | | | | | | |

^{a}The Pₐₚₚ values were calculated with Equation (7 − C).
^{b}The Pₐₚₚ of LY was greater than 0.8 × 10⁻⁶ cm/s, suggesting compromised cell monolayer integrity. The Pₐₚₚ of cladribine was excluded from the calculation of the average.
NA: not applicable.

TABLE 67
Corrected Efflux Ratio of Cladribine and Inhibition
by TUDCA and PB and Ko143 (BCRP-MDCK Cells)

| | Corrected Efflux | Percent |
|---|---|---|
| Treatment | Ratio | Inhibition |
| 10 μM Cladribine | 222 | NA |
| 10 μM Cladribine + Solution 1 | NA | NA |
| 10 μM Cladribine + Solution 2 | 3.94 | 98.2 |
| 10 μM Cladribine + Solution 3 | 20.7 | 90.7 |
| 10 μM Cladribine + Solution 4 | 159 | 28.2 |
| 10 μM Cladribine + Solution 5 | 148 | 33.2 |
| 10 μM Cladribine + Solution 6 | 263 | −18.8 |
| 10 μM Cladribine + Solution 7 | 235 | −5.81 |
| 10 μM Cladribine + Solution 8 | 198 | 10.8 |
| 10 μM Cladribine + 0.5 μM Ko143 | −0.0782 | 100 |

NA: not applicable.

The concentrations of TUDCA and PB in the dosing solutions for the BCRP IC50 assessment in C2BBe1 cells were determined. The measured concentrations of each component in at least two of three replicates were in the range of 80-120% of the nominal value in each case, indicating that the preparation of test article dosing solutions was acceptable. The detailed annotation of Solution 1 through Solution 8 is described in Table 39. The preparation of cladribine was also acceptable.

In the absence of TUDCA and PB or Ko143, the efflux ratio of cladribine was 27.6, and the addition of Ko143 decreased it to 1.73 (Table 68), corresponding to 97.2% inhibition (Table 69). These results indicate the normal function of BCRP in the test system.

In the presence of TUDCA and PB, the efflux ratio of cladribine ranged from 3.20 to 25.5 (Table 68). The inhibition of BCRP was 46.1% in the presence of Solution 3 (222 μM TUDCA and 1790 μM PB), and it was 78.0% in the presence of Solution 2 (667 μM TUDCA and 5371 M PB) (Table 69). Therefore, the estimated IC50 toward BCRP would be between 222 and 667 kM TUDCA, with 1790 and 5371 μM PB, respectively.

Currently, for orally administered drugs, the FDA use the following ratio to assess the in vitro BCRP inhibitor potential: [I]₂ of the test article over its calculated IC₅₀. Correspondingly, if [I]₂ over IC50 is less than 10, an in vivo drug-drug interaction study with a BCRP substrate is not needed.

The estimated [I]₂ of TUDCA and PB is 8005 and 64447 μM (Table 28). The projected ratio is [8005/(222˜667)] for TUDCA and [64447/(1790-5371)] for PB in C2BBe1 cells. The ratio would be greater than 10 for both TUDCA and PB.

An in vivo drug-drug interaction of AMX0035 with a BCRP substrate may be needed based on the current data.

### P-gp and BCRP Substrate Assessment

The concentrations of TUDCA and PB in the dosing solutions for the P-gp and BCRP substrate assessment in C2BBe1 cells were determined. The measured concentrations of each component in at least two of three replicates were in the range of 80-120% of the nominal value in each case, indicating that the preparation of test article dosing solutions was acceptable. The detailed annotation of Solution 1 through Solution 4 is described in Table 41. The bidirectional permeability of Solutions 1, 3, and 4 was run with one batch of C2BBe1 cells, and the bidirectional permeability of Solution 2 with another batch. The preparation of digoxin and cladribine was also acceptable.

When the bidirectional permeability of the probe substrates was conducted with Solutions 1, 3, and 4, the efflux ratio of digoxin and cladribine was 41.6 and 27.0, respectively; when the bidirectional permeability of the probe substrates was conducted with Solution 2, the efflux ratio of digoxin and cladribine was 38.2 and 19.7, respectively (Table 70). These results indicate the normal function of P-gp and BCRP in the test system.

TABLE 68
Permeability and Recovery of Cladribine in the
Absence and Presence of TUDCA and PB or Ko143
in C2BBe1 Cells (BCRP IC50 Assessment)

| | | AP-to-BL | | BL-to-AP | | |
|---|---|---|---|---|---|---|
| | | Pₐₚₚ | Recov- | Pₐₚₚ | Recov- | |
| | | (10⁻⁶ | ery | (10⁻⁶ | ery | Efflux |
| Treatment | Replicate | cm/s)^{a} | (%) | cm/s)^{a} | (%) | Ratio |
| 10 μM | 1 | 0.372 | 92.7 | 10.1 | 113 | 27.6 |
| Cladribine | 2 | 0.390 | 92.8 | 11.0 | 107 | |
| | Average | 0.381 | 92.8 | 10.5 | 110 | |
| 10 μM | 1 | 0.376 | 82.1 | 10.2 | 92.9 | 25.5 |
| Cladribine + | 2 | 0.395 | 91.1 | 9.46 | 91.5 | |
| Solution 8 | Average | 0.386 | 86.6 | 9.85 | 92.2 | |
| 10 μM | 1 | 0.437 | 86.3 | 8.46 | 106 | 25.2 |
| Cladribine + | 2 | 0.315 | 94.7 | 10.5 | 114 | |
| Solution 7 | Average | 0.376 | 90.5 | 9.47 | 110 | |
| 10 μM | 1 | 0.421 | 89.4 | 8.73 | 91.5 | 22.8 |
| Cladribine + | 2 | 1.19^{b} | 91.5 | 10.4 | 99.0 | |
| Solution 6 | Average | NA | 90.5 | 9.59 | 95.3 | |
| 10 μM | 1 | 0.352 | 82.0 | 8.92 | 83.3 | 24.2 |
| Cladribine + | 2 | 0.375 | 88.7 | 8.66 | 87.2 | |
| Solution 5 | Average | 0.363 | 85.4 | 8.79 | 85.3 | |
| 10 μM | 1 | 0.331 | 80.6 | 7.81 | 87.4 | 15.8 |
| Cladribine + | 2 | 0.643 | 86.0 | 7.60 | 87.5 | |
| Solution 4 | Average | 0.487 | 83.3 | 7.71 | 87.4 | |
| 10 μM | 1 | 0.339 | 89.4 | 5.41 | 88.0 | 15.3 |
| Cladribine + | 2 | 0.368 | 94.4 | 5.44 | 94.3 | |
| Solution 3 | Average | 0.354 | 91.9 | 5.42 | 91.1 | |
| 10 μM | 1 | 0.336 | 74.4 | 2.28 | 79.9 | 6.87 |
| Cladribine + | 2 | 0.391 | 78.4 | 2.72 | 88.1 | |
| Solution 2 | Average | 0.363 | 76.4 | 2.50 | 84.0 | |
| 10 μM | 1 | 0.406 | 84.9 | 1.32 | 82.0 | 3.20 |
| Cladribine + | 2 | 0.372 | 83.2 | 1.17 | 84.7 | |
| Solution 1 | Average | 0.389 | 84.0 | 1.25 | 83.4 | |
| 10 μM | 1 | 0.720 | 83.5 | 1.14 | 85.8 | 1.73 |
| Cladribine + | 2 | 0.749 | 83.1 | 1.41 | 91.8 | |
| 0.5 μM | Average | 0.734 | 83.3 | 1.27 | 88.8 | |
| Ko143 | | | | | | |

^{a}The Pₐₚₚ values were calculated with Equation (7 − C).
^{b}The Pₐₚₚ of LY was greater than 0.8 x 10⁻⁶ cm/s, suggesting compromised cell monolayer integrity. The Pₐₚₚ of cladribine was excluded from the calculation of the average.
NA: not applicable.

TABLE 69
Corrected Efflux Ratio of Cladribine and Inhibition
by TUDCA and PB and Ko143 (C2BBel Cells)

| | Corrected Efflux | Percent |
|---|---|---|
| Treatment | Ratio | Inhibition |
| 10 μM Cladribine | 26.6 | 0 |
| 10 μM Cladribine + Solution 1 | 2.20 | 91.7 |
| 10 μM Cladribine + Solution 2 | 5.87 | 78.0 |
| 10 μM Cladribine + Solution 3 | 14.3 | 46.1 |
| 10 μM Cladribine + Solution 4 | 14.8 | 44.2 |
| 10 μM Cladribine + Solution 5 | 23.2 | 12.8 |
| 10 μM Cladribine + Solution 6 | 21.8 | 18.1 |
| 10 μM Cladribine + Solution 7 | 24.2 | 9.09 |
| 10 μM Cladribine + Solution 8 | 24.5 | 7.81 |
| 10 μM Cladribine + 0.5 μM Ko143 | 0.733 | 97.2 |

TABLE 70
Permeability and Recovery of Digoxin and Cladribine
(P-gp and BCRP Substrate Assessment)

| | | AP-to-BL | | BL-to-AP | | |
|---|---|---|---|---|---|---|
| | | Pₐₚₚ | Recov- | Pₐₚₚ | Recov- | |
| | | (10⁻⁶ | ery | (10⁻⁶ | ery | Efflux |
| Treatment | Replicate | cm/s)^{c} | (%) | cm/s)^{c} | (%) | Ratio |
| 10 μM | 1 | 0.234 | 82.4 | 11.2 | 83.1 | 41.6 |
| Digoxin^{a} | 2 | 0.326 | 90.9 | 12.1 | 84.1 | |
| | 3 | 0.350 | 95.3 | 14.6 | 94.9 | |
| | Average | 0.303 | 89.5 | 12.6 | 87.4 | |
| | SD | 0.0612 | 6.60 | 1.76 | 6.56 | |
| 10 μM | 1 | 0.370 | 95.3 | 14.1 | 92.9 | 27.0 |
| Cladribine^{a} | 2 | 0.549 | 92.0 | 13.3 | 92.8 | |
| | 3 | 0.580 | 91.0 | 13.0 | 93.5 | |
| | Average | 0.500 | 92.8 | 13.5 | 93.1 | |
| | SD | 0.113 | 2.23 | 0.580 | 0.379 | |
| 10 μM | 1 | 0.323 | 77.9 | 13.5 | 89.5 | 38.2 |
| Digoxin^{b} | 2 | 0.381 | 92.4 | 12.1 | 96.7 | |
| | 3 | 0.298 | 88.7 | 12.8 | 90.2 | |
| | Average | 0.334 | 86.3 | 12.8 | 92.1 | |
| | SD | 0.0426 | 7.52 | 0.702 | 3.96 | |
| 10 μM | 1 | 0.778 | 82.2 | 12.8 | 97.9 | 19.7 |
| Cladribine^{b} | 2 | 0.612 | 87.6 | 12.4 | 91.2 | |
| | 3 | 0.554 | 86.8 | 13.1 | 93.0 | |
| | Average | 0.648 | 85.5 | 12.8 | 94.0 | |
| | SD | 0.116 | 2.93 | 0.366 | 3.46 | |

^{a}Used with Sol_1, Sol_3, and Sol_4.
^{b}Used with Sol_2.
^{c}The Pₐₚₚ values were calculated with Equation (7 − C).

Currently, the approach of determining the P-gp and BCRP substrate potential of a test article is as follows: if the efflux ratio (determined with Equation 9) is greater than or equal to 2.00, the results indicate a possible substrate of the transporter. The bidirectional permeability of the test article would be further determined in the presence of a transporter-specific inhibitor, and if the inhibition is greater than 50%, the test article is a substrate of the transporter.

The efflux ratio of TUDCA and PB was less than 2.00 at all tested concentrations (Table 71 and Table 72). Because the results do not meet the criteria, neither TUDCA nor PB is P-gp or BCRP substrate.

TABLE 71
Permeability and Recovery of TUDCA (P-gp
and BCRP Substrate Assessment)

| | | AP-to-BL | | BL-to-AP | | |
|---|---|---|---|---|---|---|
| | | Pₐₚₚ | Recov- | Pₐₚₚ | Recov- | |
| | | (10⁻⁶ | ery | (10⁻⁶ | ery | Efflux |
| Treatment | Replicate | cm/s)^{a} | (%) | cm/s)^{a} | (%) | Ratio |
| 81.0 μM | 1 | 6.40 | 105 | 2.74 | 100 | 0.506 |
| TUDCA | 2 | 5.84 | 101 | 2.66 | 101 | |
| (Solution 1) | 3 | 5.53 | 103 | 3.58 | 101 | |
| | Average | 5.92 | 103 | 3.00 | 101 | |
| | SD | 0.442 | 2.15 | 0.511 | 0.525 | |
| 16.2 μM | 1 | 1.19 | 71.6 | 0.553 | 76.1 | 0.305 |
| TUDCA | 2 | 2.71 | 86.8 | 0.714 | 91.6 | |
| (Solution 2) | 3 | 2.08 | 82.3 | 0.561 | 86.6 | |
| | Average | 2.00 | 80.2 | 0.610 | 84.8 | |
| | SD | 0.764 | 7.78 | 0.0906 | 7.90 | |
| 1.62 μM | 1 | 9.59 | 78.5 | 1.75 | 79.5 | 0.230 |
| TUDCA | 2 | 7.25 | 82.7 | 2.15 | 85.8 | |
| (Solution 3) | 3 | 8.12 | 79.6 | 1.84 | 79.6 | |
| | Average | 8.32 | 80.2 | 1.91 | 81.6 | |
| | SD | 1.18 | 2.21 | 0.212 | 3.59 | |
| 0.810 μM | 1 | 7.47 | 71.4 | 1.94 | 79.7 | 0.302 |
| TUDCA | 2 | 6.30 | 78.5 | 2.01 | 84.4 | |
| (Solution 4) | 3 | 7.56 | 82.4 | 2.48 | 84.8 | |
| | Average | 7.11 | 77.4 | 2.14 | 83.0 | |
| | SD | 0.702 | 5.61 | 0.295 | 2.82 | |

^{a}The Pₐₚₚ values were calculated with Equation (7 − A).

TABLE 72
Permeability and Recovery of PB (P-gp
and BCRP Substrate Assessment)

| | | AP-to-BL | | BL-to-AP | | |
|---|---|---|---|---|---|---|
| | | Pₐₚₚ | Recov- | Pₐₚₚ | Recov- | |
| | | (10⁻⁶ | ery | (10⁻⁶ | ery | Efflux |
| Treatment | Replicate | cm/s)^{a} | (%) | cm/s)^{a} | (%) | Ratio |
| 1479 μM | 1 | 23.1 | 94.3 | 25.5 | 99.0 | 0.740 |
| TUDCA | 2 | 46.2^{b} | 83.2 | 19.2 | 97.9 | |
| (Solution 1) | 3 | 29.3 | 107 | 28.2 | 94.8 | |
| | Average | 32.8 | 94.8 | 24.3 | 97.3 | |
| | SD | 11.9 | 11.8 | 4.61 | 2.17 | |
| 148 μM | 1 | 28.0 | 58.1 | 8.06 | 93.2 | 0.620 |
| TUDCA | 2 | 18.5 | 68.5 | 13.1 | 55.7 | |
| (Solution 2) | 3 | 18.3 | 56.1 | 19.1 | 70.1 | |
| | Average | 21.6 | 60.9 | 13.4 | 73.0 | |
| | SD | 5.53 | 6.65 | 5.53 | 18.9 | |
| 73.9 μM | 1 | 27.2 | 103 | 23.4 | 109 | 0.940 |
| TUDCA | 2 | 26.9 | 98.3 | 19.7 | 103 | |
| (Solution 3) | 3 | 20.5 | 97.1 | 27.0 | 94.5 | |
| | Average | 24.9 | 99.6 | 23.4 | 102 | |
| | SD | 3.74 | 3.33 | 3.68 | 7.10 | |
| 14.8 μM | 1 | 18.8 | 93.7 | 36.6 | 103 | 1.65 |
| TUDCA | 2 | 19.1 | 91.3 | 29.3 | 98.0 | |
| (Solution 4) | 3 | 19.3 | 93.8 | 28.9 | 100 | |
| | Average | 19.1 | 92.9 | 31.6 | 100 | |
| | SD | 0.249 | 1.44 | 4.33 | 2.56 | |

^{a}The Pₐₚₚ values were calculated with Equation (7 − A).
^{b}The Pₐₚₚ values were calculated with Equation (7 − B).

In summary, in vitro, neither TUDCA nor PB is a substrate of P-gp or BCRP. The estimated IC50 of TUDCA toward each transporter is as follows: 0.0741˜0.222 μM (OAT1), greater than 162 μM (OCT2), 54.0˜162 μM (OAT3), 12.9˜38.7 μM (OATP1B1), 38.7˜116 μM (OATP1B3), 270˜810 μM (MATE1), greater than 810 μM (MATE2K), 889˜2668 μM (P-gp in MDR1-MDCK cells), 222˜667 μM (P-gp in C2BBe1 cells), 296˜889 μM (BCRP in BCRP-MDCK cells), and 222˜667 μM (BCRP in C2BBe1 cells). The estimated IC50 of PB toward each transporter is as follows: 6.76˜20.3 μM (OAT1), greater than 14785 μM (OCT2), 4928˜14785 μM (OAT3), 1208˜3625 μM (OATP1B1), 3625˜10875 μM (OATP1B3), 24642˜73927 μM (MATE1), greater than 73927 μM (MATE2K), 7161˜21482 μM (P-gp in MDR1-MDCK cells), 1790˜5371 μM (P-gp in C2BBe1 cells), 2387˜7161 μM (BCRP in BCRP-MDCK cells), and 1790˜5371 μM (BCRP in C2BBe1 cells).

### Example 7— Drug-Drug Interaction Study of Tenofovir Disoproxil Fumarate and AMX0035

The potential drug-drug interaction of Tenofovir Disoproxil Fumarate (TDF) and AMX0035 was evaluated. TDF is a prodrug that is rapidly converted into the active moiety, Tenofovir, in plasma. Tenofovir is a known substrate for the organic anion transporter OAT1 (SLC22A6) in the kidney and this study was designed to determine whether co-administration of AMX0035 alters the plasma or urine concentrations of Tenofovir in the rat.

This study was comprised of one group of three male CD® (Sprague-Dawley) IGS rats. Rats were administered 25 mg/kg/dose TDF once daily on Days 1 through 4 via PO gavage at a dose volume of 5 mL/kg in order to achieve a steady state concentration of Tenofovir prior to administration of AMX0035. On Day 5, rats were administered an 840 mg/kg/dose of AMX0035 via PO gavage at a dose volume of 9.94 mL/kg followed by administration of a 25 mg/kg/dose TDF via PO gavage at a dose volume of 5 mL/kg. The dose of Tenofovir was administered 1.5 hours after AMX0035 which was designed to approximate the Tₘₐₓ of AMX0035.

Clinical observations were recorded approximately 1 hour post-dose on Days 1-4 and twice on Day 5, once post-AMX0035 administration and once post-TDF administration. Body weight measurements were recorded prior to randomization and then daily through the end of the study. Plasma samples were collected from all animals at 0.25, 0.5, 1, 2, 4, 8, and 24 hours post-dose on Days 1 and 5 (Day 5 collections occurred following TDF administration) for analysis of systemic exposure to Tenofovir, TDF, PB, and TUDCA. Urine samples were collected from all animals at 0-4, 4-8, and 8-24 hours post-dose on Days 1 and 5 (Day 5 collections occurred following TDF administration) for calculations of urine recovery of Tenofovir, TDF, PB, and TUDCA.

Results showed that all rats survived until the scheduled sacrifice on Day 6. No test-article-related changes in body weight occurred, and no test article-related clinical observations were noted. Tenofovir was rapidly formed upon systemic absorption of TDF. No prodrug (TDF) was detected in plasma at any time point following administration on either Day 1 or Day 5. Mean peak (Cₘₐₓ) and total (AUCₗₐₛₜ) plasma exposures to Tenofovir increased 1.24- and 2.17-fold, respectively, in the presence of AMX0035. The Cₘₐₓ plasma levels of both TUDCA (298 ng/mL) and PB (10,200 ng/mL) detected in this study exceeded the IC₅₀ values reported for OAT1 inhibition in a previous in vitro transporter study in terms of total drug concentrations. These results are consistent with AMX0035 producing a modest increase in Tenofovir exposure (AUCₗₐₛₜ) that is mediated by an inhibitory effect on OAT1.

### Introduction

The objective of this study was to assess if AMX0035 is an inhibitor of the OAT1 transporter in rats. To do this, the study evaluated the drug-drug interaction of Tenofovir disoproxil fumarate (TDF), a known substrate for organic anion transporter OAT1, and AMX0035. TDF was administered via PO gavage for four consecutive days to achieve a steady state concentration prior to administering AMX0035 via PO gavage on Day 5. Following AMX0035 administration on Day 5, TDF was administered at 1.5 hours post-AMX0035 dose.

### Methods

### Experimental Design

The purpose of this study was to evaluate drug-drug interaction of TDF and AMX0035 following oral administration. TDF was administered once daily for five days. AMX0035 was administered once on Day 5 followed by administration of TDF at 1.5 hours post-AMX0035 dose.

Pharmacokinetic Evaluation Pharmacokinetic analyses were performed on the individual plasma concentration versus time data for Tenofovir, PB, and TUDCA using Phoenix WinNonlin non-compartmental analysis (linear trapezoidal rule for AUC calculations). Nominal dose values and nominal sampling times were used for calculations. Any concentrations reported as BLQ (<50 ng/mL) were set equal to zero. For calculations of AUC on Study Days 1 and 5, the plasma levels of Tenofovir, PB, and TUDCA at time zero were set equal to zero. PK parameters were not evaluated for PB and TDF on Day 1 and for TDF on Day 5 because the plasma concentration values were BLQ. Pharmacokinetic analysis included the determination of Cₘₐₓ, Tₘₐₓ, AUCₗₐₛₜ, Tₗₐₛₜ, and AUC₀₋₂₄.

Individual and mean Tenofovir, TDF, PB, and TUDCA plasma concentrations versus time data were presented with standard deviation (SD), reported to three significant figures, and percent coefficient of variation (CV %), reported to one decimal place. Pharmacokinetic parameters were presented as individual and mean values. Individual Tₘₐₓ and Tₗₐₛₜ were reported to two significant figures, while all other values were reported to three significant figures. Mean Tenofovir, PB, and TUDCA PK parameters, except for Tₘₐₓ and Tₗₐₛₜ, were presented to three significant figures and CV % to one decimal place. Mean Tₘₐₓ and Tₗₐₛₜ values were presented to two significant figures.

Total urine recovery data for Tenofovir, TDF, and PB was calculated for the 24 hour collection period on Days 1 and 5. Urine concentrations for PB analysis in all animals on Day 1, for TUDCA analysis in all animals on Days 1 and 5, and for TDF analysis in two animals on Day 1 and one animal on Day 5 were BLQ (<20 ng/mL) so no recovery data was calculated. The reported urine recovery data included amount recovered (ng) and percent recovered (%) for TDF only. Individual and mean Tenofovir, TDF, and PB urine recovery data were presented with SD, reported to three significant figures, and CV %, reported to one decimal place.

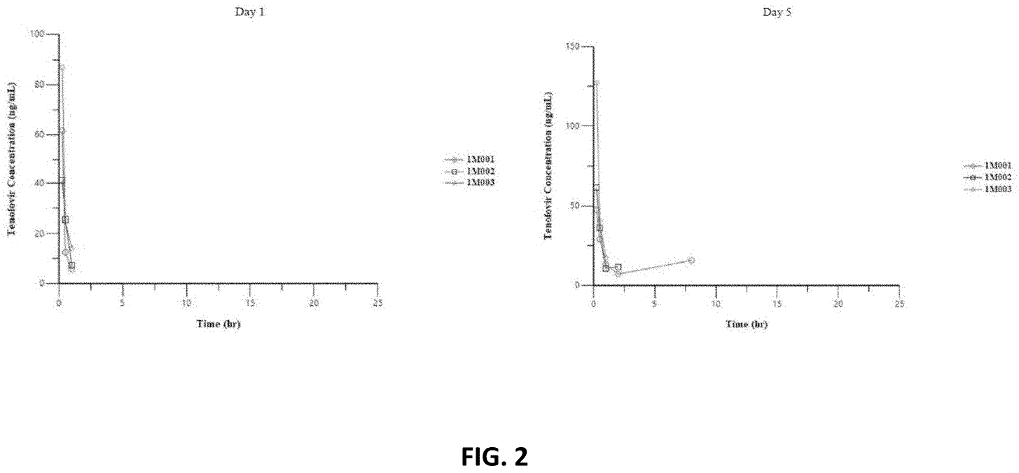

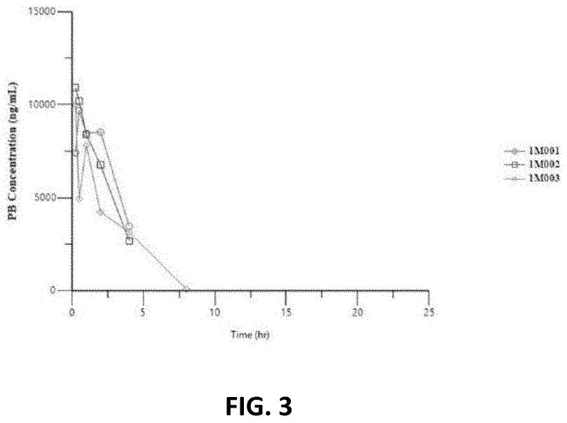

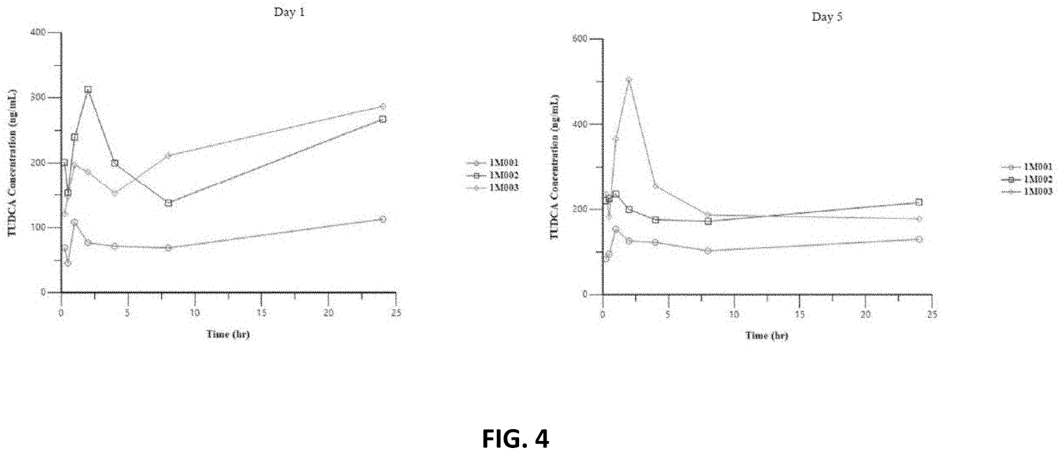

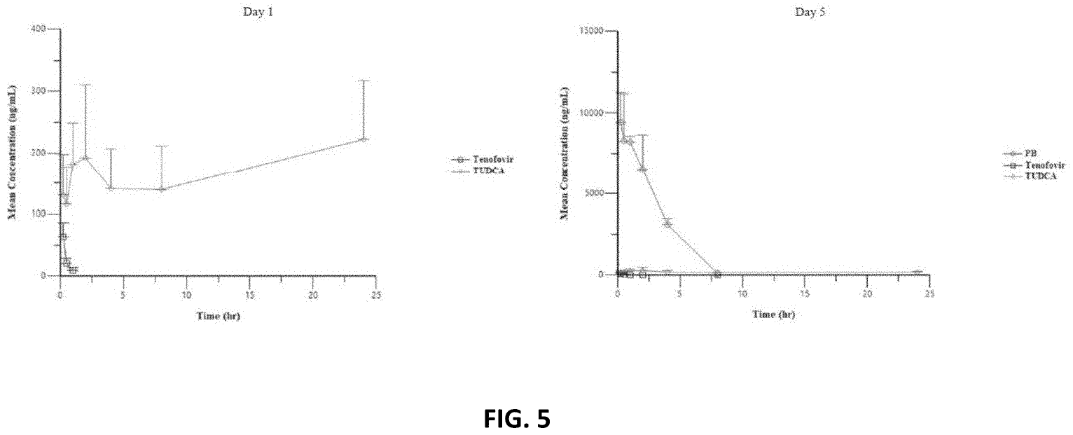

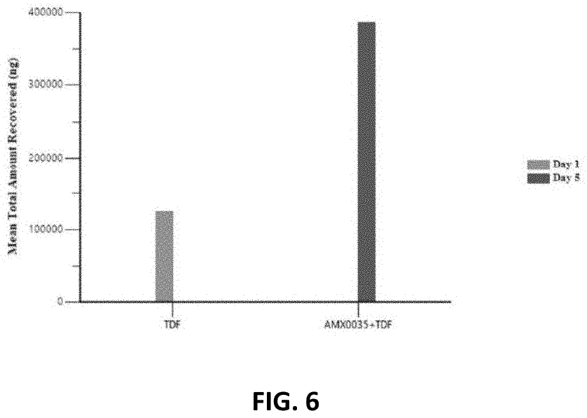

Individual and mean Tenofovir, TDF, PB, and TUDCA plasma concentration versus time data are shown in Tables 73-76 and illustrated in FIGS. 2-5. Individual animal and mean Tenofovir, PB, and TUDCA PK parameters in male Sprague Dawley Rats are presented in Tables 77-78. Individual and mean Tenofovir, TDF, and PB total urine recovery data are shown in Tables 79-80 and illustrated in FIG. 6.

Pharmacokinetic Abbreviations:
AUC: Area under the concentration-time curve
AUC₀₋₂₄ Area under the curve from the time of dosing to the time of the last measurable concentration
AUCₗₐₛₜ: Area under the curve from the time of dosing to the time of the last measurable concentration
BLQ: Below the limit of quantitation
Cₘₐₓ: The maximum observed concentration
CV %: Percent coefficient of variation
N: Number of observations
NA: Not Applicable
SD: Standard Deviation
Tₗₐₛₜ: Time of the last point with quantifiable concentration
Tₘₐₓ: The time at which Cₘₐₓ occurred

TABLE 73
Individual and Mean Tenofovir, TDF, PB, and TUDCA Plasma Concentration (ng/mL) versus Time Data Following
Oral Administration of TDF only (Day 1) or AMX0035 and TDF (Day 5) in Male Sprague Dawley Rats

| | | Dose Level | | | Animal | Time (hr) | | | | | | |
|---|---|---|---|---|---|---|---|---|---|---|---|---|
| Analyte | Day | (mg/kg) | Treatment | Sex | ID | 0.25 | 0.50 | 1.0 | 2.0 | 4.0 | 8.0 | 24 |
| PB | 1 | 25 | TDF | Male | 1M001 | BLQ | BLQ | BLQ | BLQ | BLQ | BLQ | BLQ |
| | | | | | 1M002 | BLQ | BLQ | BLQ | BLQ | BLQ | BLQ | BLQ |
| | | | | | 1M003 | BLQ | BLQ | BLQ | BLQ | BLQ | BLQ | BLQ |
| | | | | | N | 3 | 3 | 3 | 3 | 3 | 3 | 3 |
| | | | | | Mean | 0.00 | 0.00 | 0.00 | 0.00 | 0.00 | 0.00 | 0.00 |
| | | | | | SD | 0.00 | 0.00 | 0.00 | 0.00 | 0.00 | 0.00 | 0.00 |
| | | | | | CV % | NA | NA | NA | NA | NA | NA | NA |
| PB | 5 | 840/25 | AMX0035 + | Male | 1M001 | 7400 | 9680 | 8450 | 8510 | 3470 | BLQ | BLQ |
| | | | TDF | | 1M002 | 10900 | 10200 | 8380 | 6750 | 2680 | BLQ | BLQ |
| | | | | | 1M003 | 9980 | 4940 | 7810 | 4230 | 3150 | 102 | BLQ |
| | | | | | N | 3 | 3 | 3 | 3 | 3 | 3 | 3 |
| | | | | | Mean | 9430 | 8270 | 8210 | 6500 | 3100 | 34.0 | 0.00 |
| | | | | | SD | 1810 | 2900 | 351 | 2150 | 397 | 58.9 | 0.00 |
| | | | | | CV % | 19.2 | 35.0 | 4.3 | 33.1 | 12.8 | 173.2 | NA |

TABLE 74
Individual and Mean Tenofovir, TDF, PB, and TUDCA Plasma Concentration
(ng/mL) versus Time Data Following Oral Administration of TDF only
(Day 1) or AMX0035 and TDF (Day 5) in Male Sprague Dawley Rats

| | | Dose Level | | | Animal | Time (hr) | | | | | | |
|---|---|---|---|---|---|---|---|---|---|---|---|---|
| Analyte | Day | (mg/kg) | Treatment | Sex | ID | 0.25 | 0.50 | 1.0 | 2.0 | 4.0 | 8.0 | 24 |
| TDF | 1 | 25 | TDF | Male | 1M001 | BLQ | BLQ | BLQ | BLQ | BLQ | BLQ | BLQ |
| | | | | | 1M002 | BLQ | BLQ | BLQ | BLQ | BLQ | BLQ | BLQ |
| | | | | | 1M003 | BLQ | BLQ | BLQ | BLQ | BLQ | BLQ | BLQ |
| | | | | | N | 3 | 3 | 3 | 3 | 3 | 3 | 3 |
| | | | | | Mean | 0.00 | 0.00 | 0.00 | 0.00 | 0.00 | 0.00 | 0.00 |
| | | | | | SD | 0.00 | 0.00 | 0.00 | 0.00 | 0.00 | 0.00 | 0.00 |
| | | | | | CV % | NA | NA | NA | NA | NA | NA | NA |
| TDF | 5 | 840/25 | AMX0035 + | Male | 1M001 | BLQ | BLQ | BLQ | BLQ | BLQ | BLQ | BLO |
| | | | TDF | | 1M002 | BLQ | BLQ | BLQ | BLQ | BLQ | BLQ | BLQ |
| | | | | | 1M003 | BLQ | BLQ | BLQ | BLQ | BLQ | BLQ | BLQ |
| | | | | | N | 3 | 3 | 3 | 3 | 3 | 3 | 3 |
| | | | | | Mean | 0.00 | 0.00 | 0.00 | 0.00 | 0.00 | 0.00 | 0.00 |
| | | | | | SD | 0.00 | 0.00 | 0.00 | 0.00 | 0.00 | 0.00 | 0.00 |
| | | | | | CV % | NA | NA | NA | NA | NA | NA | NA |

TABLE 75
Individual and Mean Tenofovir, TDF, PB, and TUDCA Plasma Concentration (ng/mL) versus Time Data Following
Oral Administration of TDF only (Day 1) or AMX0035 and TDF (Day 5) in Male Sprague Dawley Rats

| | | Dose Level | | | Animal | Time (hr) | | | | | | |
|---|---|---|---|---|---|---|---|---|---|---|---|---|
| Analyte | Day | (mg/kg) | Treatment | Sex | ID | 0.25 | 0.50 | 1.0 | 2.0 | 4.0 | 8.0 | 24 |
| Tenofovir | 1 | 25 | TDF | Male | 1M001 | 61.6 | 12.6 | 5.94 | BLQ | BLQ | BLQ | BLQ |
| | | | | | 1M002 | 41.3 | 25.6 | 7.61 | BLQ | BLQ | BLQ | BLQ |
| | | | | | 1M003 | 87.0 | 25.1 | 14.2 | BLQ | BLQ | BLQ | BLQ |
| | | | | | N | 3 | 3 | 3 | 3 | 3 | 3 | 3 |
| | | | | | Mean | 63.3 | 21.1 | 9.25 | 0.00 | 0.00 | 0.00 | 0.00 |
| | | | | | SD | 22.9 | 7.37 | 4.37 | 0.00 | 0.00 | 0.00 | 0.00 |
| | | | | | CV % | 36.2 | 34.9 | 47.2 | NA | NA | NA | NA |
| Tenofovir | 5 | 840/25 | AMX0035 + | Male | 1M001 | 47.6 | 29.0 | 13.4 | 7.29 | BLQ | 15.8 | BLQ |
| | | | TDF | | 1M002 | 61.3 | 36.3 | 10.9 | 11.8 | BLQ | BLQ | BLQ |
| | | | | | 1M003 | 127 | 40.7 | 17.5 | BLQ | BLQ | BLQ | BLQ |
| | | | | | N | 3 | 3 | 3 | 3 | 3 | 3 | 3 |
| | | | | | Mean | 78.6 | 35.3 | 13.9 | 6.36 | 0.00 | 5.27 | 0.00 |
| | | | | | SD | 42.4 | 5.91 | 3.33 | 5.95 | 0.00 | 9.12 | 0.00 |
| | | | | | CV % | 54.0 | 16.7 | 23.9 | 93.6 | NA | 173.2 | NA |

TABLE 76
Individual and Mean Tenofovir, TDF, PB, and TUDCA Plasma Concentration
(ng/mL) versus Time Data Following Oral Administration of TDF only
(Day 1) or AMX0035 and TDF (Day 5) in Male Sprague Dawley Rats

| | | Dose Level | | | Animal | Time (hr) | | | | | | |
|---|---|---|---|---|---|---|---|---|---|---|---|---|
| Analyte | Day | (mg/kg) | Treatment | Sex | ID | 0.25 | 0.50 | 1.0 | 2.0 | 4.0 | 5.0 | 24 |
| TUDCA | 1 | 25 | TDF | Male | 1M001 | 69.3 | 45.4 | 108 | 76.8 | 71.4 | 68.8 | 113 |
| | | | | | 1M002 | 201 | 154 | 239 | 313 | 199 | 138 | 267 |
| | | | | | 1M003 | 121 | 148 | 197 | 186 | 153 | 211 | 287 |
| | | | | | N | 3 | 3 | 3 | 3 | 3 | 3 | 3 |
| | | | | | Mean | 130 | 116 | 181 | 192 | 141 | 139 | 222 |
| | | | | | SD | 66.4 | 61.0 | 66.9 | 118 | 64.6 | 71.1 | 95.2 |
| | | | | | CV % | 50.9 | 52.7 | 36.9 | 61.6 | 45.8 | 51.1 | 42.8 |
| TUDCA | 5 | 840/25 | AMX0035 + | Male | 1M001 | 84.0 | 96.1 | 154 | 126 | 123 | 103 | 130 |
| | | | TDF | | 1M002 | 220 | 225 | 236 | 200 | 176 | 172 | 216 |
| | | | | | 1M003 | 235 | 183 | 366 | 505 | 255 | 187 | 178 |
| | | | | | N | 3 | 3 | 3 | 3 | 3 | 3 | 3 |
| | | | | | Mean | 180 | 168 | 252 | 277 | 185 | 154 | 175 |
| | | | | | SD | 83.2 | 65.7 | 107 | 201 | 66.4 | 44.8 | 43.1 |
| | | | | | CV % | 46.3 | 39.1 | 42.4 | 72.5 | 36.0 | 29.1 | 24.7 |

Note:
BLQ = below the limit of quantitation (<50 ng/mL)

TABLE 77
Individual and Mean Tenofovir, PB, and TUDCA PK Parameters Following Oral Administration
of TDF only (Day 1) or AMX0035 and TDF (Day 5) in Male Sprague Dawley Rats

| | | Dose Level | | | Animal | Cₘₐₓ | Tₘₐₓ | AUCₗₐₛₜ | Tₗₐₛₜ | AUC₀₋₂₄ |
|---|---|---|---|---|---|---|---|---|---|---|
| Analyte | Day | (mg/kg) | Treatment | Sex | ID | (ng/mL) | (hr) | (hr*ng/ml) | (hr) | (hr*ng/mL) |
| PB | 5 | 840/25 | AMX0035 + | Male | 1M001 | 9680 | 0.50 | 28100 | 4.0 | 35000 |
| | | | TDF | | 1M002 | 10900 | 0.25 | 25600 | 4.0 | 31000 |
| | | | | | 1M003 | 9980 | 0.25 | 26200 | 8.0 | 27000 |
| | | | | | N | 3 | 3 | 3 | 3 | 3 |
| | | | | | Mean | 10200 | 0.33 | 26600 | 5.3 | 31000 |
| | | | | | SD | 636 | 8.14 | 1260 | 2.3 | 3990 |
| | | | | | CV % | 6.2 | 43.3 | 4.7 | 43.3 | 12.9 |
| Tenofovir | 1 | 25 | TDF | Male | 1M001 | 61.6 | 0.25 | 21.6 | 1.0 | 24.6 |
| | | | | | 1M002 | 41.3 | 0.25 | 21.8 | 1.0 | 25.6 |
| | | | | | 1M003 | 87.0 | 0.25 | 34.7 | 1.0 | 41.8 |
| | | | | | N | 3 | 3 | 3 | 3 | 3 |
| | | | | | Mean | 63.3 | 0.25 | 26.1 | 1.0 | 30.7 |
| | | | | | SD | 22.9 | 0.0 | 7.50 | 0.0 | 9.66 |
| | | | | | CV % | 36.2 | 0.0 | 28.8 | 0.0 | 31.5 |
| Tenofovir | 5 | 840/25 | AMX0035 + | Male | 1M001 | 47.6 | 0.25 | 75.4 | 8.0 | 202 |
| | | | TDP | | 1M002 | 61.3 | 0.25 | 43.0 | 2.0 | 54.8 |
| | | | | | 1M003 | 127 | 0.25 | 51.4 | 1.0 | 60.1 |
| | | | | | N | 3 | 3 | 3 | 3 | 3 |
| | | | | | Mean | 78.6 | 0.25 | 56.6 | 3.7 | 106 |
| | | | | | SD | 42.4 | 0.0 | 16.8 | 3.8 | 83.3 |
| | | | | | CV % | 54.0 | 0.0 | 29.7 | 103.3 | 78.9 |

TABLE 78
Individual and Mean Tenofovir, PB, and TUDCA PK Parameters Following Oral Administration
of TDF only (Day 1) or AMX0035 and TDF (Day 5) in Male Sprague Dawley Rats

| | | Dose Level | | | Animal | Cₘₐₓ | Tₘₐₓ | AUCₗₐₛₜ | Tₗₐₛₜ | AUC₀₋₂₄ |
|---|---|---|---|---|---|---|---|---|---|---|
| Analyte | Day | (mg/kg) | Treatment | Sex | ID | (ng/mL) | (hr) | (hr*ng/mL) | (hr) | (hr*ng/mL) |
| TUDCA | 1 | 25 | TDF | Male | 1M001 | 113 | 24 | 2040 | 24 | 2040 |
| | | | | | 1M002 | 313 | 2.0 | 4870 | 24 | 4870 |
| | | | | | 1M003 | 287 | 24 | 5380 | 24 | 5380 |
| | | | | | N | 3 | 3 | 3 | 3 | 3 |
| | | | | | Mean | 238 | 17 | 4090 | 24 | 4090 |
| | | | | | SD | 109 | 13 | 1800 | 0.0 | 1800 |
| | | | | | CV % | 45.8 | 76.2 | 44.0 | 0.0 | 44.0 |
| TUDCA | 5 | 840/25 | AMX0035 + | Male | 1M001 | 154 | 1.0 | 2800 | 24 | 2800 |
| | | | TDF | | 1M002 | 236 | 1.0 | 4590 | 24 | 4590 |
| | | | | | 1M003 | 505 | 2.0 | 5220 | 24 | 5220 |
| | | | | | N | 3 | 3 | 3 | 3 | 3 |
| | | | | | Mean | 298 | 1.3 | 4200 | 24 | 4200 |
| | | | | | SD | 184 | 0.58 | 1250 | 0.0 | 1250 |
| | | | | | CV % | 61.5 | 43.3 | 29.9 | 0.0 | 29.9 |

Note:
PK parameters were not evaluated for PB on Day 1 and TDF on Days 1 and 5 because concentrations values were BLQ.

TABLE 79
Individual and Mean Tenofovir, TDF, and PB Total Urine Recovery Data Following Oral Administration
of TDF only (Day 1) or AMX0035 and TDF (Day 5) in Male Sprague Dawley Rats

| | | Dose Level | | | Animal | Amount Recovered | Percent Recovered |
|---|---|---|---|---|---|---|---|
| Analyte | Day | (mg/kg) | Treatment | Sex | ID | (ng) | (%) |
| PB | 5 | 840/25 | AMX0035 + | Male | 1M001 | 2080 | NA |
| | | | TDF | | 1M002 | 5160 | NA |
| | | | | | 1M003 | 405 | NA |
| | | | | | N | 3 | 0 |
| | | | | | Mean | 2550 | NA |
| | | | | | SD | 2410 | NA |
| | | | | | CV % | 94.6 | NA |
| TDF | 1 | 25 | TDF | Male | 1M002 | 11.3 | 0.000156 |
| | | | | | N | 1 | 1 |
| | | | | | Mean | 11.3 | 0.000156 |
| | | | | | SD | NA | NA |
| | | | | | CV % | NA | NA |
| TDF | 5 | 840/25 | AMX0035 + | Male | 1M002 | 12.2 | 0.000156 |
| | | | TDF | | 1M003 | 4340 | 0.0535 |
| | | | | | N | 2 | 2 |
| | | | | | Mean | 2180 | 0.0268 |
| | | | | | SD | NA | NA |
| | | | | | CV % | NA | NA |

TABLE 80
Individual and Mean Tenofovir, TDF, and PB Total Urine Recovery Data Following Oral Administration
of TDF only (Day 1) or AMX0035 and TDF (Day 5) in Male Sprague Dawley Rats

| | | Dose Level | | | Animal | Amount Recovered | Percent Recovered |
|---|---|---|---|---|---|---|---|
| Analyte | Day | (mg/kg) | Treatment | Sex | ID | (ng) | (%) |
| Tenofovir | 1 | 25 | TDF | Male | 1M001 | 63200 | NA |
| | | | | | 1M002 | 234000 | NA |
| | | | | | 1M003 | 77300 | NA |
| | | | | | N | 3 | 0 |
| | | | | | Mean | 125000 | NA |
| | | | | | SD | 95100 | NA |
| | | | | | CV % | 76.0 | NA |
| Tenofovir | 5 | 840/25 | AMX0035 + | Male | 1M001 | 764000 | NA |
| | | | TDF | | 1M002 | 296000 | NA |
| | | | | | 1M003 | 99500 | NA |
| | | | | | N | 3 | 0 |
| | | | | | Mean | 386000 | NA |
| | | | | | SD | 341000 | NA |
| | | | | | CV % | 88.3 | NA |

Note:
Urine concentrations for PB analysis in all animals on Day 1, for TUDCA analysis in all animals on Days 1 and 5, and for TDF analysis in two animals on Day 1 and one animal on Day 5 were BLQ (<20 ng/mL) so no recovery data was calculated.

Following a single oral administration of 25 mg/kg/dose TDF on Day 1 and 840 mg/kg/dose AMX0035+25 mg/kg/dose TDF on Day 5, quantifiable TDF plasma concentrations were not observed in any animals on either sampling day as TDF was rapidly converted to the active component, Tenofovir.

Quantifiable Tenofovir concentrations were observed in all animals by the first collection time at 0.25 hours post-dose. Individual time to peak (Tₘₐₓ) for Tenofovir occurred at 0.25 hours post-dose on both Day 1 and Day 5. The mean Cₘₐₓ for Tenofovir on Day 1 was 63.3 ng/mL and the compound was rapidly cleared from the plasma compartment with no detectable compound (BLQ) after 1 hour on Day 1 and after 2 hours on Day 5. In the presence of AMX0035, however, both the mean Cₘₐₓ and AUCₗₐₛₜ of Tenofovir increased by 1.24 and 2.17 fold, respectively. The mean plasma concentrations of Tenofovir were higher at each measurable time point (1.24-, 1.67- and 1.5-fold at the 0.25, 0.5 and 1 hour time points, respectively) in the presence of AMX0035.

TUDCA is an endogenous bile acid and thus was quantifiable on Day 1, in the absence of any dose of AMX0035. Mean endogenous levels of TUDCA measured on Day 1 ranged from 130-222 ng/mL over the 24 hour time course. Following dosing with AMX0035 on Day 5, individual Tₘₐₓ for TUDCA occurred 1 to 2 hours post-dose. Mean plasma concentrations for TUDCA on Day 5 ranged from 154 ng/mL to 277 ng/mL and were generally 30-50% higher at each time point than on Day 1, reflecting the dosing of AMX0035. The levels of TUDCA in plasma remained fairly constant over the entire time course.

Quantifiable PB plasma concentrations were observed on Day 5 at 0.25 hours post-dose of AMX0035 and generally remained quantifiable through 4 hours. PB was rapidly absorbed and individual Tₘₐₓ for PB occurred at 0.25 to 0.5 hours post-dose. Similar to Tenofovir, PB was rapidly cleared and concentrations were not detectable following the 4 hour time point.

A previous study provided IC50 values for in vitro inhibition of human OAT1 by both TUDCA and PB. These values were 74-222 nM (35-111 ng/mL) for TUDCA and 6.76-20.3 μM (1103-3313 ng/mL) for PB. The peak (Cmax) plasma values observed in this study for both TUDCA (298 ng/mL) and PB (10,200) exceeded the IC50 range for OAT1 inhibition.

Three of the four analytes were detected in the urine. As expected, TUDCA was not detected in any urine samples as bile acids are cleared through the liver. Although samples were collected over three time points (0-4, 4-8, and 8-24 hours) on both Day 1 and Day 5, no urine was produced by any animals for the 4-8 hour time point on Day 1. In addition, one of three animals produced no urine for the 0-4 hour time point. Thus, four of nine potential samples on Day 1 had no sample volume to use for measurement. This is problematic due to the rapid clearance of Tenofovir and the need to compare data from Day 1 with Day 5. The concentrations in samples from these early time points would likely by high and would significantly impact the total amount recovered in the urine.

### CONCLUSION

Tenofovir was rapidly formed upon systemic absorption of TDF. No prodrug (TDF) was detected in plasma at any time point following administration on either Day 1 or Day 5. Mean peak (Cₘₐₓ) and total (AUCₗₐₛₜ) plasma exposures to Tenofovir increased 1.24- and 2.17-fold, respectively, in the presence of AMX0035. The Cₘₐₓ plasma levels of both TUDCA (298 ng/mL) and PB (10,200 ng/mL) detected in this study exceeded the IC50 values reported for OAT1 inhibition in a previous in vitro transporter study (See Example 6) in terms of total drug concentrations. These results are consistent with AMX0035 producing a modest increase in Tenofovir exposure (AUCₗₐₛₜ) that is mediated by an inhibitory effect on OAT1.

1-55. (canceled)

56. A method of treating at least one symptom of Amyotrophic Lateral Sclerosis (ALS) in a subject, the method comprising:
(a) administering to a subject who has received a first dosage of a substrate of a cytochrome P450 (CYP) a composition comprising about 1 gram of Taurursodiol (TURSO) and about 3 grams of sodium phenylbutyrate,
(b) determining or having determined a first level of the substrate or a metabolite thereof in a first biological sample from the subject, and
(c) administering to the subject a second dosage of the substrate, wherein the second dosage is lower than the first dosage.

57. The method of claim 56, wherein the CYP is CYP1A2, CYP2B6, CYP2C8, CYP2C9, CYP2C19, CYP2D6, or CYP3A.

58. The method of claim 56, wherein the CYP is CYP2C9, and wherein the substrate is warfarin.

59. The method of claim 56, wherein the CYP is CYP3A, and wherein the substrate is alfentanil, cyclosporine, or quinidine.

60. The method of claim 56, wherein the CYP is CYP3A4.

61. The method of claim 56, wherein the substrate of the CYP is mexiletine, alfentanil, quinidine, cyclosporine, or warfarin.

62. The method of claim 56, wherein the substrate of the CYP has a narrow therapeutic index.

63.-74. (canceled)

75. A method of treating at least one symptom of ALS in a subject who has received a first dosage of a substrate of Organic Anion Transporter 1 (OAT1), the method comprising:
(a) determining or having determined a first level of the substrate or a metabolite thereof in a first biological sample from the subject;
(b) administering to the subject a composition comprising TURSO and sodium phenylbutyrate;
(c) determining or having determined a second level of the substrate or the metabolite thereof in a second biological sample from the subject, wherein the second level is higher than the first level; and
(d) administering to the subject a second dosage of the substrate, wherein the second dosage is lower than the first dosage.

76. The method of claim 75, wherein the substrate of OAT1 is a penicillin, non-steroidal anti-inflammatory drug (NSAID), HIV protease inhibitor, or an antiviral drug.

77. The method of claim 76, wherein the NSAID is diclofenac, ketoprofen, or methotrexate.

78. The method of claim 76, wherein the substrate of OAT1 is an antiviral drug, and the antiviral drug is Adefovir, Cidofovir, or Tenofovir.

79. The method of claim 75, further comprising step (e), determining or having determined a third level of the substrate or the metabolite thereof in a third biological sample from the subject.

80. The method of claim 79, wherein the third level of the substrate or the metabolite thereof is lower than the second level.

81. The method of claim 75, wherein the composition is administered to the subject orally or through a feeding tube.

82. The method of claim 75, wherein the subject is diagnosed with ALS.

83. The method of claim 75, wherein the subject is suspected as having ALS.

84. The method of claim 75, wherein the composition is administered once a day.

85. The method of claim 75, wherein the composition is administered twice a day.

86. The method of claim 75, wherein step (b) comprises administering to the subject about 1 gram of TURSO and about 3 grams of sodium phenylbutyrate once a day.

87. The method of claim 75, wherein step (b) comprises administering to the subject about 1 gram of TURSO and about 3 grams of sodium phenylbutyrate twice a day.

88. A method of administering a composition comprising TURSO and sodium phenylbutyrate to a subject who has received a first dosage of a substrate of OAT1, the method comprising:
(a) determining or having determined a first level of the substrate or a metabolite thereof in a first biological sample from the subject;
(b) administering to the subject a composition comprising TURSO and sodium phenylbutyrate;
(c) determining or having determined a second level of the substrate or the metabolite thereof in a second biological sample from the subject, wherein the second level is higher than the first level; and
(d) administering to the subject a second dosage of the substrate, wherein the second dosage is lower than the first dosage.

89. The method of claim 88, wherein the substrate of OAT1 is a penicillin, non-steroidal anti-inflammatory drug (NSAID), HIV protease inhibitor, or an antiviral drug.

90. The method of claim 89, wherein the substrate of OAT1 is an antiviral drug, and the antiviral drug is Adefovir, Cidofovir, or Tenofovir.

91. The method of claim 88, further comprising step (e), determining or having determined a third level of the substrate or the metabolite thereof in a third biological sample from the subject.

92. The method of claim 91, wherein the third level of the substrate or the metabolite thereof is lower than the second level.

93. The method of claim 88, wherein the composition is administered to the subject orally or through a feeding tube.

94. The method of claim 88, wherein the composition is administered once a day.

95. The method of claim 88, wherein the composition is administered twice a day.

96. The method of claim 88, wherein step (b) comprises administering to the subject about 1 gram of TURSO and about 3 grams of sodium phenylbutyrate once a day.

97. The method of claim 88, wherein step (b) comprises administering to the subject about 1 gram of TURSO and about 3 grams of sodium phenylbutyrate twice a day.
